(19) United States
(12) Patent Application Publication
(10) Pub. No.: US 2012/0065614 A1
(43) Pub. Date: Mar. 15, 2012

(54) POLYIONIC TRANSITIONAL METAL PHOSPHORESCENT COMPLEX/POLYMER HYBRID SYSTEMS FOR BIOIMAGING AND SENSING APPLICATIONS

(75) Inventors: Mohammad A. Omary, Denton, TX (US); Zhibing Hu, Denton, TX (US); Sreekar Marpu, Denton, TX (US)

(73) Assignee: UNIVERSITY OF NORTH TEXAS, Denton, TX (US)

(21) Appl. No.: 13/228,947

(22) Filed: Sep. 9, 2011

Related U.S. Application Data
(60) Provisional application No. 61/381,928, filed on Sep. 10, 2010.

(51) Int. Cl. A61K 31/66; A61K 47/48; A61K 31/722; A61P 35/00; G01N 21/75; C12Q 1/02; A61M 37/00; A61K 31/675; A61K 49/00; B82Y 15/00

(52) U.S. Cl. 604/500; 424/78.29; 424/78.27; 514/55; 424/9.1; 436/172; 436/73; 435/29; 977/773; 977/902; 977/774

(57) ABSTRACT

A new technique to stabilize transition metal phosphors in a wide variety of stimuli-sensitive polymers and gels is disclosed herein. Other than stabilization in stimuli sensitive/biocompatible matrix some of these transition metal based phosphors are also shown to act as phosphorescent crosslinkers that physically or chemically crosslink polymeric chains to form micro/nanoparticles. The microspheres/nanospheres of the present invention show decreased size and photoluminescence enhancement with particularly high sensitization at physiological pH and temperature. The so formed phosphorescent micro/nanospheres are useful for biological or environmental applications including biological labeling, imaging, and optical sensing. The techniques in the present invention enable usage of imaging agents and sensors at very low concentrations and also minimize or eliminate the usage of toxic chemical crosslinkers typically used to synthesize polymeric micro/nanoparticles.

### CROSS-REFERENCE TO RELATED APPLICATIONS

This application is a non-provisional application of U.S. provisional patent application 61/381,928 filed on Sep. 10, 2010 and entitled “Polyionic Transitional Metal Phosphorescent Complex/Polymer Hybrid Systems for Bioimaging and Sensing Applications” which is hereby incorporated by reference in its entirety.

### STATEMENT OF FEDERALLY FUNDED RESEARCH

This invention was made with U.S. Government support under Grant Nos. CHE-0349313 and DMR-0805089 awarded by the NSF. The government has certain rights in this invention.

### TECHNICAL FIELD OF THE INVENTION

The present invention relates in general to the field of phosphorescent complexes, and more particularly to methods and compositions for stabilizing and enhancing photoluminescence properties of phosphorescent complexes of the transition metals in a wide variety of hydrogels and polymers in an aqueous environment.

### REFERENCE TO A SEQUENCE LISTING

None.

### BACKGROUND OF THE INVENTION

Without limiting the scope of the invention, its background is described in connection with phosphorescent complexes in polymers and hydrogels in an aqueous environment.

U.S. Pat. No. 6,207,077 issued to Burnell-Jones (2001) discloses luminescent polymers showing bright and long-lasting photoluminescent afterglow, strong thermostimulation of afterglow by heat and electroluminescent properties comprising thermosetting unsaturated polyester resins. The polymers in the U.S. Pat. No. 6,207,077 are prepared by condensing mixtures of ethylenically unsaturated and aromatic dicarboxylic acids and anhydrides with dihydric alcohols and a polymerizable vinylidene monomer. The phosphorescent pigments include alkaline earth aluminate phosphors, zinc sulfide phosphors and mixtures of these phosphors.

WIPO Patent Publication No. WO/2003/102109 (Marsitzky et al. 2003) discloses phosphorescent or luminescent conjugated polymers, whose emission is based on the phosphorescence of covalently bonded metal complexes, optionally combined with the fluorescence of the polymer chain in non-aqueous (organic) environments and the solid state. The invention also relates to a method for producing said polymers and to their use in electroluminescent assemblies.

WIPO Patent Publication No. WO/2005/021678 (Koyama et al. 2005) relates to a phosphorescent polymer compound comprising a phosphorescent monomer unit and a hole transporting monomer unit having a triphenylamine structure, and an organic light emitting device using the said polymer. The Koyama patent focuses on non-aqueous (organic) environments and the solid state. Use of the phosphorescent polymer compound as described in the Koyama patent enables production of organic light emitting device with a high light emitting efficiency at a low voltage, which is suitable for increasing the emission area and mass production.

### SUMMARY OF THE INVENTION

The present invention describes phosphorescent hybrid systems in aqueous media comprising light-emitting transition metal complexes in a wide variety of hydrogels and polymers. The hybrid systems include stimuli-sensitive phosphorescent hydrogel microspheres in which water soluble polyionic complexes are entrapped in different aqueous polymers. The stabilized phosphor molecules have been shown to exhibit up to two order of magnitude photoluminescence (PL) enhancement at different pH and temperatures, leading to PL turn on in some preferred embodiments. This work also results in the formation of first ever reported phosphorescent polymeric micro/nanoparticles from benign polymers such as chitosan where the transition metal based phosphor is acting both as the crosslinker and light emitting center. This enables the use of similar gels and similar enhancement mechanism for detecting luminescent molecules at very low concentration where emission from the species is completely unnoticeable by regular luminescence techniques.

A large variety of polyionic transition metal complexes stabilized in a large variety of polymers and hydrogels have produced bright phosphorescence in aqueous media. The preferred embodiments for the phosphorescent centers include 3- or 2-coordinate Au(I) mononuclear or dinuclear complexes with sulfonated phosphine ligands, cyclic trinuclear complexes of Au(I), Cu(I), and/or Ag(I) with carboxylated and/or aminated azolate ligands, full- or half-sandwich adducts in which various metal ions are coordinated perpendicular to the planar center of the aforementioned trinuclear complexes, and Ru(II) octahedral complexes with neutral or carboxylated polypyridyl ligands. The preferred embodiments for the aqueous polymer stabilizers include poly N-isopropylacrylamide (PNIPAM) hydrogels with carboxylated and aminated co-polymer moieties, chitosan, agarose, PAA (poly acrylic acid), PVA (poly vinyl alcohol), PAMAM (Polyamidoamine), PEG (Poly ethyleneglycol), alginic acid, HPC (hydroxyl propylcellulose), or a combination thereof.

In one embodiment the present invention describes a stimulus-responsive water soluble hybrid phosphorescent system comprising: one or more polyanionic or polycationic transition metal based phosphors in the form of a complex, a coordination compound, or combinations thereof and a stimulus-responsive matrix comprising a polymer, a hydrogel, a colloid, a microgel, or combinations thereof, wherein the matrix has a charge that is opposite to the polyanionic or polycationic metal based phosphor, wherein the one or more metal based phosphors are linked, attached or entrapped in the matrix to form of one or more luminescent polymeric nano or micro particles in the matrix in the presence or absence of a chemical crosslinking agent. In one aspect the one or more transition metal based phosphors is coordinated by one or more substituted or unsubstituted phosphine ligands, one or more azolate ligands including substituted or unsubstituted pyrazolate, triazolate, imidazolate, or combination thereof or by one or more polyimine ligands including substituted or unsubstituted 2,2′-bipyridine, 1,10-phenanthroline, 2,2′:6′,2″-terpyridine, or combination thereof.

In one aspect of the composition disclosed above the one or more transition metal based phosphors have a general formula given by:

[A^{x+}]ₙ₁[(M)ₙ₂(L)ₙ₃]^{y-}

wherein A^{x+} comprises Na⁺, K⁺, Cs⁺, NH₄⁺, R₄N⁺ wherein R is selected from hydrogen or alkyl, Mg⁺², Ca⁺², or combination thereof, M comprises a transition metal selected from the group consisting of gold, silver, copper, platinum, palladium, nickel, ruthenium, osmium, iridium, rhenium, or combination thereof, and at least one L comprises

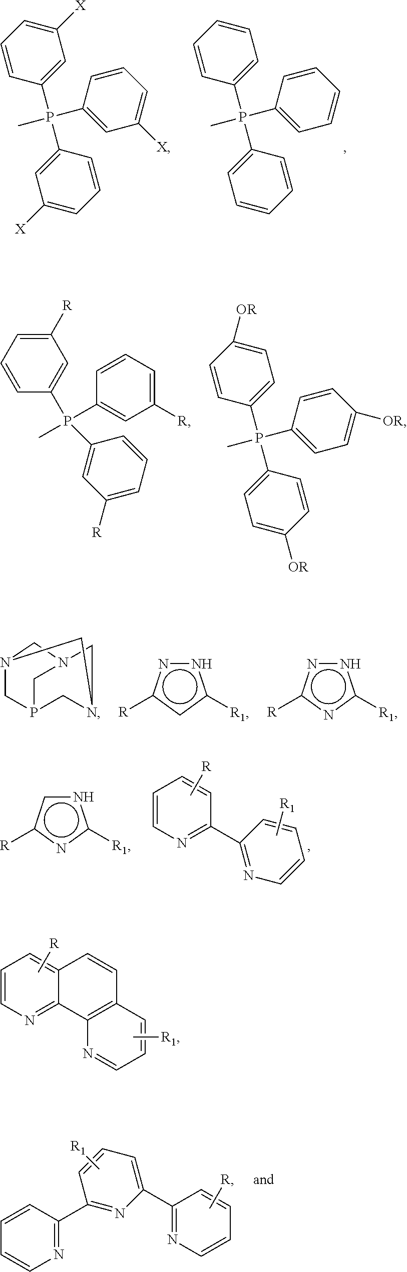

combinations and modifications thereof, wherein R and R₁ are selected from hydrogen, alkyl, alkoxy, aryl, NH₂, NH₃⁺, COOH, COO⁻, SO₃H, SO₃⁻, PO₄H₂, PO₄²⁻, OH, Cl, Br, COOR′, or R′₃N⁺ wherein R′ is selected from hydrogen or alkyl, n₁, n₂, n₃, and y are integer numbers equaling 1 or greater.

In another aspect of the composition disclosed hereinabove the one or more transition metal based phosphors have a general formula given by:

[(M)ₙ₁(L)ₙ₂]^{x+}[X^{y-}]ₙ₃

wherein X^{y-} comprises Cl⁻, Br⁻, I⁻, NO₃⁻, PO₄³⁻, CO₃²⁻, COO⁻, BF₄⁻, PF₆⁻, SO₄²⁻, SO₃²⁻, or combination thereof, M comprises a transition metal selected from the group consisting of gold, silver, copper, platinum, palladium, nickel, ruthenium, osmium, iridium, rhenium, or combination thereof, at least one L comprises

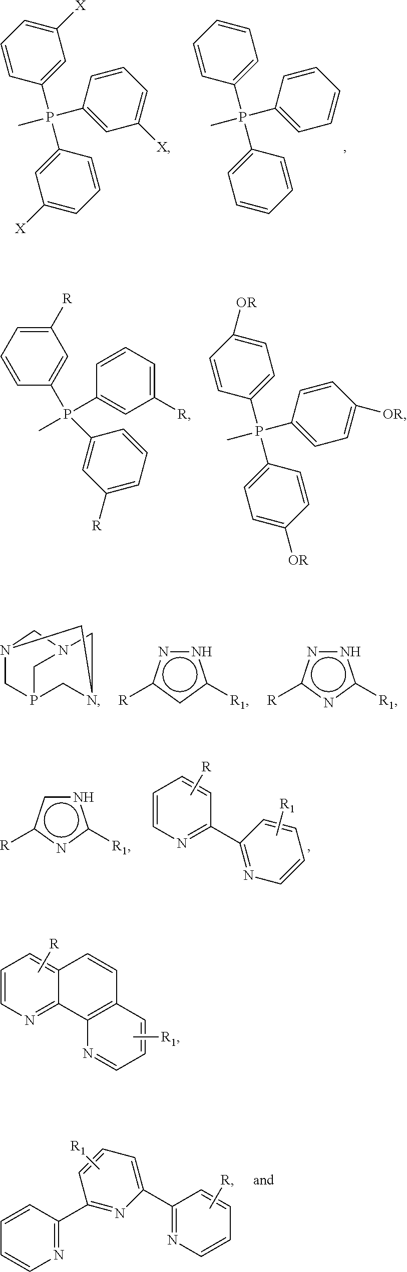

combinations and modifications thereof, wherein R and R₁ are selected from hydrogen, alkyl, alkoxy, aryl, NH₂, NH₃⁺, COOH, COO⁻, SO₃H, SO₃⁻, PO₄H₂, PO₄²⁻, OH, Cl, Br, COOR′, or R′₃N⁺ wherein R′ is selected from hydrogen or alkyl, and n₁, n₂, n₃, and x are integer numbers equaling 1 or greater.

In yet another aspect of the present invention the matrix comprises poly-N-isopropylacrylamide (PNIPAM), chemically modified PNIPAM including PNIPAM-co-allylamine and PNIPAM-co-acrylic acid, chitosan, chemically modified chitosan, poly acrylic acid (PAA), polyvinyl alcohol (PVA), alginic acid, PEG, modified PEG, agarose, hydroxy propyl cellulose, methyl methacrylate (MMA), hydroxyethyl methacrylate (HEMA), polystyrene, and poly-hydroxyethyl methacrylate. In another aspect the composition is responsive to one or more stimuli selected from the group consisting of pH, temperature, electric, magnetic, optical, and environmental stimuli. In another aspect the metal based phosphor has a formula given by [A^{x+}]ₙ₁ tris[tris(3,3′,3″-trisulfonatophenyl)phosphine]aurate(I) wherein [A^{x+}]ₙ₁ comprises [Na⁺]₈, [K⁺]₈, [Cs⁺]₈, [NH₄⁺]₈, [R₄N⁺]₈, [Mg⁺²]₄, [Ca⁺²]₄, or combination thereof.

In a specific aspect the tris[tris(3,3′,3″-trisulfonatophenyl)phosphine]aurate(I) has a structure given by:

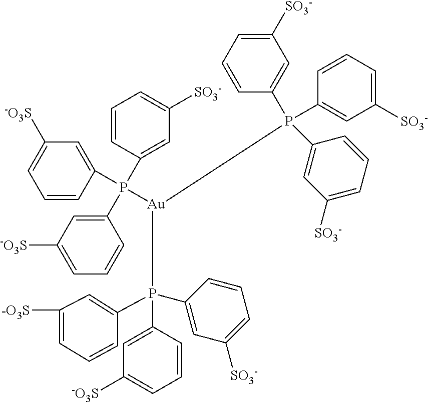

In another aspect the metal based phosphor has a formula given by [Ru(2,2′-Bipyridine)₃](PF₆)₂, [Ru(1,10-Phenanthroline)₃](PF₆)₂, [Os(2,2′-Bipyridine)₃](PF₆)₂, or [Os(1,10-Phenanthroline)₃](PF₆)₂. The [Ru(2,2′-Bipyridine)₃](PF₆)₂ or [Os(2,2′-Bipyridine)₃](PF₆)₂ disclosed herein has a structure given by:

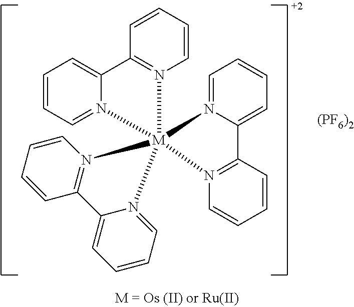

In a specific aspect the metal based phosphor has a formula given by K₄-[Ru(4,4′-dicarboxy-2,2′-bipyridine)₃], K₄[Os(4,4′-dicarboxy-2,2′-bipyridine)₃], [Ru(dicarboxy-1,10-Phenanthroline)₃](PF₆)₂, or [Os(dicarboxy-1,10-Phenanthroline)₃](PF₆)₂. The K₄-[Ru(4,4′-dicarboxy-2,2′-bipyridine)₃] or K₄[Os(4,4′-dicarboxy-2,2′-bipyridine)₃] disclosed herein has a structure given by:

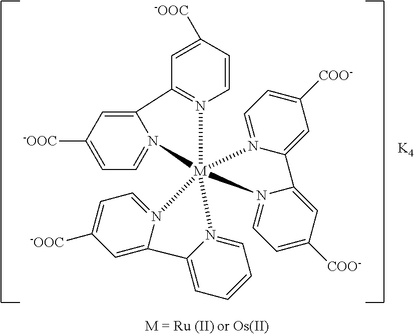

In another aspect the metal based phosphor has a formula given by K₄[Ru(4,4′,4″-tricarboxy-2,2′:6′,2″-terpyridine)₂] or K₄[Ru(4,4′,4″-tricarboxy-2,2′:6′,2″-terpyridine)₂]. The K₄[Ru(4,4′,4″-tricarboxy-2,2′:6′,2″-terpyridine)₂] or K₄[Ru(4,4′,4″-tricarboxy-2,2′:6′,2″-terpyridine)₂] has a structure given by:

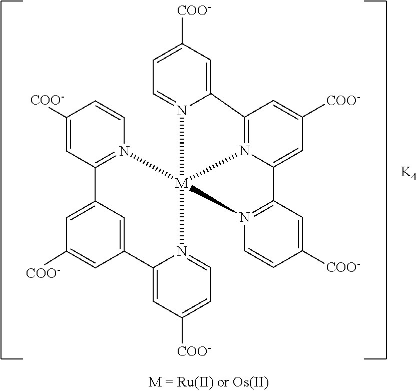

In another aspect the metal based phosphor has a structure given by:

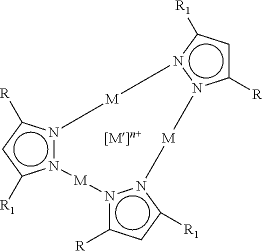

wherein R and R₁ are selected from hydrogen, alkyl, alkoxy, aryl, NH₂, NH₃⁺, COOH, COO⁻, SO₃H, SO₃⁻, PO₄H₂, PO₄²⁻, OH, Cl, Br, COOR′, or R′₃N⁺ wherein R′ is selected from hydrogen or alkyl, M comprises transition metals including gold, silver, copper, or combination thereof, and [M′]ⁿ⁺ comprises Ag⁺, Pb²⁺, Hg²⁺, and Tl³⁺.

In another aspect of the present invention the metal based phosphor has a structure given by:

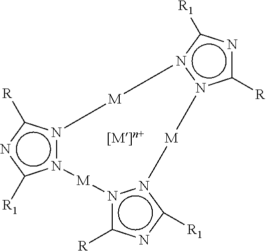

wherein R and R₁ are selected from hydrogen, alkyl, alkoxy, aryl, NH₂, NH₃⁺, COOH, COO⁻, SO₃H, SO₃⁻, PO₄H₂, PO₄²⁻, OH, Cl, Br, COOR′, or R′₃N⁺ wherein R′ is selected from hydrogen or alkyl, M comprises transition metals including gold, silver, copper, or combination thereof, and [M′]ⁿ⁺ comprises Ag⁺, Pb²⁺, Hg²⁺, and Tl³⁺.

In yet another aspect the metal based phosphor has a structure given by:

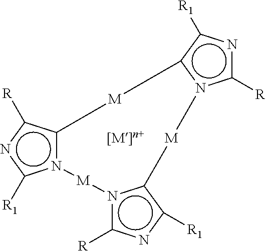

wherein R and R₁ are selected from hydrogen, alkyl, alkoxy, aryl, NH₂, NH₃⁺, COOH, COO⁻, SO₃H, SO₃⁻, PO₄H₂, PO₄²⁻, OH, Cl, Br, COOR′, or R′₃N⁺ wherein R′ is selected from hydrogen or alkyl, M comprises transition metals including gold, silver, copper, or combination thereof, and [M′]ⁿ⁺ comprises Ag⁺, Pb²⁺, Hg²⁺, and Tl³⁺.

In one aspect the matrix is poly-N-isopropylacrylamide (PNIPAM), chemically modified PNIPAM including PNIPAM-co-allylamine and PNIPAM-co-acrylic acid, or a combination thereof. In another aspect the matrix is chitosan, a chitosan derivative, a modified chitosan, or combinations thereof. In a related aspect the chitosan derivatives comprise succinyl chitosan, octanoyl chitosan, quateraminated chitosan, caproyl chitosan, myristoyl chitosan, palmitoyl chitosan, chitosan thioglycolic acid, phosphorylated chitosan, carboxy methyl chitosan, and thiol containing chitosan. In another aspect the composition of the instant invention is used for biosensing and bioimaging, in anti-tumor therapy, for drug delivery, in biomedical devices, in live cell imaging, in environmental monitoring, in toxic metal removal, in small molecule detection, and for biological and chemical recognition. In yet another aspect the polymeric nano or microparticles have sizes ranging from 20 nm-1000 nm and the polymeric nano or microparticles can be incorporated in a delivery system along with biologically benign polymers (comprising chitosan, alginic acid, or combinations and modifications thereof), wherein the biologically benign polymers comprise one or more functional groups to reduce a toxicity, enhance a specificity or both. In another aspect related to the composition disclosed herein a surface positive or negative charge of the composition is easily controllable by a selection and use of different polymers. This aspect is particularly important as the surface charge of the composition controls a cellular uptake. In another aspect the particle size of the composition ranging from nano to micron size provides an enhanced cellular uptake by one or more mechanisms, wherein the luminescence of the polymeric nano or microparticles provides for enhanced monitoring of the cellular uptake.

In one aspect of the instant invention the composition comprises both hydrophilic and hydrophobic segments to entrap one or more photosensitizers, wherein the photosensitizers on light exposure are excited to a triplet state thereby leading to a generation of a singlet oxygen (¹O₂) or free radicals used for photodynamic therapy. In a specific aspect the composition has an emission maximum of 525 nm and an excitation maximum of 292 nm in aqueous solution and an emission maximum of 500 nm and an excitation maximum of 356 nm in a solid state. In another aspect of the composition disclosed hereinabove the composition comprising Pt(II) based phosphorescent polymeric nano/microparticles are used for oxygen sensing applications.

In another embodiment the instant invention relates to a stimulus-responsive water soluble phosphorescent system comprising: a polyanionic metal based phosphor comprising [A^{x+}]ₙ₁ tris[tris(3,3′,3″-trisulfonatophenyl)phosphine]aurate(I) wherein [A^{x+}]ₙ₁ comprises [Na⁺]₈, [K⁺]₈, [Cs⁺]₈, [NH₄⁺]₈, [R₄N⁺]₈, [Mg⁺²]₄, [Ca⁺²]₄, or combination thereof, and tris[tris(3,3′,3″-trisulfonatophenyl)phosphine]aurate(I) having a structure given by;

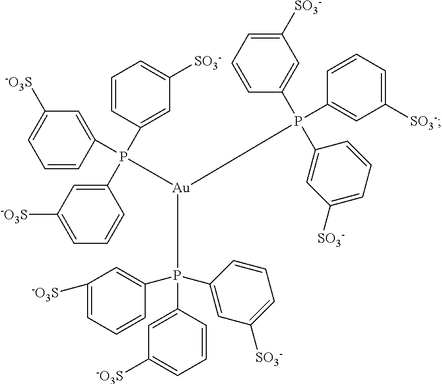

and
a stimulus-responsive matrix comprising poly-N-isopropylacrylamide (PNIPAM), chemically modified PNIPAM including PNIPAM-co-allylamine and PNIPAM-co-acrylic acid, chitosan, or a combination thereof, wherein the [A^{x+}]ₙ₁ tris[tris(3,3′,3″-trisulfonatophenyl)phosphine]aurate(I) is entrapped in the matrix to form of one or more luminescent polymeric nanoparticles or crosslinked to form polymeric micro/nanoparticles in absence of any chemical crosslinker.

In one aspect the composition is responsive to one or more stimuli selected from the group consisting of pH, temperature, electric, magnetic, optical, and environmental stimuli. In another aspect the matrix is chitosan, a chitosan derivative, a modified chitosan, or combinations thereof. In yet another aspect the chitosan derivatives comprise succinyl chitosan, octanoyl chitosan, quateraminated chitosan, caproyl chitosan, myristoyl chitosan, palmitoyl chitosan, chitosan thioglycolic acid, phosphorylated chitosan, carboxy methyl chitosan, and thiol containing chitosan. In another aspect the composition is used for biosensing and bioimaging, in anti-tumor therapy, for drug delivery, in biomedical devices, in tissue scaffolds, for cellular encapsulation, in live cell imaging, in environmental monitoring, in toxic metal detection or removal, in small molecule detection, and for biological and chemical recognition. A key feature of the composition disclosed hereinabove is that there is no typical chemical crosslinking agent used along with a minimization of the use of harmful/toxic typical chemical crosslinkers. In specific aspects the composition is a liquid at room temperature and the composition comprising the PNIPAM matrix undergoes a transition from a liquid to a gel or an aggregate upon an increase in temperature. Gelation or aggregation from PNIPAM particles/microgel in presence of a transitional metal based phosphor is novel in itself.

In yet another embodiment the present invention discloses a stimulus-responsive water soluble phosphorescent system comprising: a Ru(II) or Os(II) based phosphor comprising a structure selected from the group consisting of

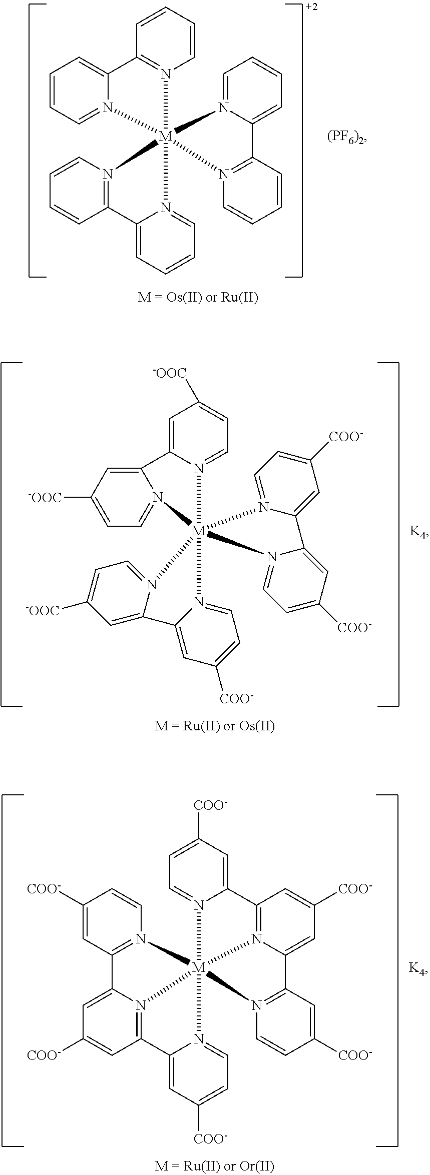

and combinations and modifications thereof, and a stimulus-responsive matrix comprising poly-N-isopropylacrylamide (PNIPAM), chemically modified PNIPAM including PNIPAM-co-allylamine and PNIPAM-co-acrylic acid, or a combination thereof, chitosan, or a combination thereof, wherein the Ru(II) or Os(II) based phosphor is entrapped in the matrix to form of one or more luminescent polymeric nanoparticles or crosslinked to form polymeric micro/nanoparticles in absence of any chemical crosslinker.

In one aspect the composition disclosed above is responsive to one or more stimuli selected from the group consisting of pH, temperature, electric, magnetic, optical, and environmental stimuli. In another aspect the matrix is chitosan, a chitosan derivative, a modified chitosan, or combinations thereof. In yet another aspect the chitosan derivatives comprise succinyl chitosan, octanoyl chitosan, quateraminated chitosan, caproyl chitosan, myristoyl chitosan, palmitoyl chitosan, chitosan thioglycolic acid, phosphorylated chitosan, carboxy methyl chitosan, and thiol containing chitosan. The composition disclosed hereinabove is used for biosensing and bioimaging, in anti-tumor therapy, for drug delivery, in biomedical devices, in tissue scaffolds, for cellular encapsulation, in live cell imaging, in environmental monitoring, in toxic metal removal, in small molecule detection, and for biological and chemical recognition.

In one embodiment the present invention provides a method for making a stimulus-responsive hybrid luminescent or phosphorescent system comprising the steps of: mixing an aqueous solution comprising a stimulus-responsive matrix comprising a polymer, a hydrogel, a colloid, a microgel, or combinations thereof with an aqueous solution of polyionic metal based phosphor with agitation to form a mixture in an inert atmosphere, centrifuging the mixture with removal of a separated supernatant and a washing of a sediment at one or more regular intervals, and forming the stimulus-responsive hybrid phosphorescent system by incubation of the centrifuged mixture in water, wherein the incubation results in a formation of one or more metal loaded microgel colloids or crystals.

The method disclosed hereinabove comprises the optional steps of: (i) crosslinking the formed microgels by using one or more crosslinking agents to form a crosslinked microgel network and (ii) freeze drying the microgels or the microgel network. In one aspect the metal based phosphors comprise anionic or cationic Au(I) or Pt(II) based phosphors in the form of a complex, a coordination compound, or combinations thereof. In another aspect the matrix is poly-N-isopropylacrylamide (PNIPAM), chemically modified PNIPAM including PNIPAM-co-allylamine and PNIPAM-co-acrylic acid, or a combination thereof. In yet another aspect the system is used for biosensing and bioimaging, in anti-tumor therapy, for drug delivery, in biomedical devices, in tissue scaffolds, for cellular encapsulation, in live cell imaging, in environmental monitoring, in toxic metal removal, in small molecule detection, and for biological and chemical recognition.

In another embodiment the present invention further discloses a method for making one or more luminescent or phosphorescent polyelectrolyte nano or microparticles comprising the steps of: (i) providing a polymer solution comprising modified or unmodified polymers wherein the polymers are selected from the group consisting of poly-N-isopropylacrylamide (PNIPAM), chemically modified PNIPAM including PNIPAM-co-allylamine and PNIPAM-co-acrylic acid, or a combination thereof, chitosan, chemically modified chitosan, chitosan derivatives poly acrylic acid (PAA), polyvinyl alcohol (PVA), alginic acid, PEG, modified PEG, agarose, hydroxy propyl cellulose, methyl methacrylate (MMA), hydroxyethyl methacrylate (HEMA), polystyrene, and poly-hydroxyethyl methacrylate, (ii) adding a solution of one or more polyionic metal based phosphors to the polymer solution to form an opalescent suspension, wherein the metal based phosphors are entrapped in the polymer, (iii) centrifuging the opalescent suspension, and (iv) filtering the centrifuged suspension to recover the luminescent or phosphorescent polyelectrolyte nano or microparticles and separate any unreacted polymers or any unentrapped metal based phosphors. In one aspect of the method the polyionic metal based phosphors comprise anionic or cationic transition or noble metal based phosphors in the form of a complex, a coordination compound, or combinations thereof, wherein the metals are selected from the group consisting of gold, silver, copper, platinum, europium, terbium, ruthenium, rhenium, iridium, thallium, and osmium. In another aspect of the method the luminescent or phosphorescent polyelectrolyte nano or microparticles are used for biosensing and bioimaging, in anti-tumor therapy, for drug delivery, in biomedical devices, in tissue scaffolds, for cellular encapsulation, in live cell imaging, in environmental monitoring, in toxic metal removal, in small molecule detection, and for biological and chemical recognition.

In yet another embodiment the instant invention discloses a method for forming a polymer stabilized cyclic phosphorescent systems comprising the steps of: (i) mixing a solution of a cyclic ligand with a metal, metal complex or coordination compound in presence of an amine or a base with stirring to form a mixture, wherein the ligand comprises a modified or unmodified pyrazole, triazole, imidazole compound, or combinations and modifications thereof, (ii) exposing the mixture to light, air or both, (iii) following a progression of the reaction by monitoring a change in an emissive color of the mixture, (iv) filtering and recrystallizing the solution at a completion of the reaction, (v) adding the filtered and recrystallized metal comprising cyclic ligand to a modified or unmodified polyionic polymer with stirring, wherein the polymer is a biopolymer, a thermosensitive polymer or both, wherein the metal comprising cyclic ligand is entrapped and stabilized in the polymer and (vi) recovering a polymer stabilized cyclic phosphorescent system by centrifugation.

The instant invention in one embodiment also provides for a method for forming a polymer stabilized cyclic phosphorescent systems comprising the steps of: adding a solution of a cyclic ligand to a modified or unmodified polyionic polymer with stirring at a controlled pH to form a mixture, wherein the pH is controlled by an addition of an acid or a base, wherein the ligand comprises a modified or unmodified pyrazole, triazole, imidazole compound, or combinations and modifications thereof, wherein the polymer is a biopolymer, a thermosensitive polymer, or both, adding a metal, metal complex or coordination compound with stirring to the mixture, following a progression of the reaction by monitoring a change in an emissive color of the mixture, and centrifuging the mixture at a completion of the reaction to separate any unreacted polymer or the metal and to recover the polymer stabilized cyclic phosphorescent system in the sediment. In one aspect the metal comprises gold, silver, platinum, or copper. In another aspect the system is used for monitoring or detecting a level of one or more toxic heavy metal contaminants in a liquid sample.

The instant invention in various embodiments describes multiple applications of the stimulus-responsive water soluble hybrid phosphorescent systems. In one embodiment the invention discloses a method of treating a cancer in a human subject comprising the steps of: identifying the human subject in need for the treatment of the cancer, and administering a therapeutically effective amount of a composition comprising one or more polyanionic or polycationic metal based phosphors in the form of a complex, a coordination compound, or combinations thereof linked, attached or entrapped in a stimulus-responsive matrix comprising a polymer, a hydrogel, a colloid, a softgel, a microgel, or combinations thereof in an amount sufficient to treat the cancer, wherein the composition is administered alone, as a combination with other anticancer drugs, or in conjunction with a chemotherapeutic or radiation regimen. The treatment method further comprises the optional steps of: measuring or monitoring a photoluminescent signal emanating from the metal based phosphors to follow an uptake of the composition into one or more normal or cancerous cells and increasing a local concentration of the composition or the combination anticancer drug by inducing an aggregation, a gelation or both of the composition by providing localized heating or a hyperthermia treatment. In one aspect the composition has a gelation temperature at or about a body temperature.

Another embodiment of the instant invention describes a method for monitoring or detecting a level of one or more volatile organic compounds (VOCs), carcinogens, and other contaminants in a sample comprising the steps of: providing the sample suspected of having the VOCs, carcinogens, or other contaminants, wherein the sample is a liquid or a gas, and comprises water, biological fluids, air, or a mixture of gases, providing a photoluminescent composition comprising one or more polyanionic or polycationic metal based phosphors in the form of a complex, a coordination compound, or combinations thereof linked, attached or entrapped in a stimulus-responsive matrix comprising a polymer, a hydrogel, a colloid, a softgel, a microgel, measuring a baseline photoluminescent signal following contact of the composition with a pure sample, wherein the pure sample does not have the VOCs, carcinogens, or other contaminants, measuring the photoluminescent signal following contact of the composition with the sample suspected of having the VOCs, carcinogens, or other contaminants, and monitoring or detecting the level of one or more volatile organic compounds (VOCs), carcinogens, and other contaminants by measuring a change in an intensity and a magnitude of the photoluminescent signal induced by a change in a pH or a temperature of the sample by the one or more volatile organic compounds (VOCs), carcinogens, and other contaminants.

In yet another embodiment the instant invention discloses a method for monitoring or detecting a level of one or more toxic heavy metal contaminants in a liquid sample comprising the steps of: (i) providing the liquid sample suspected of having the toxic metal contamination, wherein the liquid sample comprises drinking water, biological fluids, industrial effluents, ground water, wastewater, or combinations thereof, (ii) providing a photoluminescent composition comprising one or more polyanionic or polycationic metal based phosphors in the form of a complex, a coordination compound, or combinations thereof linked, attached or entrapped in a stimulus-responsive matrix comprising a polymer, a hydrogel, a colloid, a softgel, a microgel, (iii) monitoring an emission color of the composition prior to contacting the composition with the liquid sample, wherein the monitoring is done under daylight, UV light or both, (iv) monitoring the emission color after contacting the composition with the liquid sample, wherein the monitoring is done under daylight, UV light or both, (v) monitoring or detecting the level of one or more toxic metal contaminants in a liquid sample based on a change in the emission color induced by an entrapment or a change in a pH of the one or more toxic heavy metal contaminants by the composition, and (vi) monitoring an Energy Dispersive X-ray analysis (EDX) spectrum of a matrix/polymeric particle system formed after contacting the composition with the liquid contaminant sample enabling a qualitative and a quantitative determination heavy metal contaminant. In one aspect the one or more toxic heavy metal contaminants comprise, lead, mercury, thallium, arsenic, copper, and silver. In another aspect the polymers may have a film forming ability thereby enabling manufacture of a luminescent pH film with a pH sensitive emission profile.

The present invention also discloses a method for in vivo imaging one or more live cells, tissues, or both, targeting one or more receptors, cells, or tissues or detecting a level of a protein, a molecular marker, or a biomolecule comprising the steps of: providing an animal or a human subject, injecting a photoluminescent composition comprising one or more polyanionic or polycationic metal based phosphors in the form of a complex, a coordination compound, or combinations thereof linked, attached or entrapped in a stimulus-responsive matrix comprising a polymer, a hydrogel, a colloid, a softgel, a microgel, and monitoring a photoluminescent signal following the injection to image the one or more live cells, tissues, or both, target the one or more receptors, cells, or tissues, or to detect a level of the protein, the molecular marker or the biomolecule. In one aspect of the imaging method the photoluminescent composition is optionally tagged, linked or conjugated with one or more antibodies, organic dyes, ligands or nanoparticles. In another aspect an enhanced photoluminescence signal from a composition complex in presence of a stimuli responsive matrix can minimize concentration of dye or overcome an auto fluorescence signal from the one or more live cells, tissues, receptors, or any other biological matrix.

### BRIEF DESCRIPTION OF THE DRAWINGS

For a more complete understanding of the features and advantages of the present invention, reference is now made to the detailed description of the invention along with the accompanying figures and in which:

FIGS. 1A-1C are general schematic structures for a polyanionic transition metal based phosphor (1A), a specific polyanionic gold based phosphor (1B), and a poly N-isopropylacrylamide (PNIPAM) polymer (1C);

FIG. 2 is a general schematic structure of polycationic transition metal based phosphors;

FIGS. 3A and 3B show general schematic structures of polyionic cyclic trimer transition metal based phosphors: Pyrazole ligand based cyclic trimer (3A) and Triazole ligand based cyclic trimer (3B);

FIGS. 4A and 4B show general schematic structures of polyionic cyclic trimer transition metal based phosphors: Imidazole ligand based cyclic trimer (4A) and a modified Imidazole ligand by itself (4B);

FIG. 4C-4E show schematic structures of Ru and Os metal complexes: [Ru(bpy)₃](PF₆)₂ or [Os(bpy)₃](PF₆)₂ where bpy=2,2′-Bipyridine (4C), K₄[Ru(dcbpy)₃] or K₄[Os(dcbpy)₃] where dcbpy=4,4′-dicarboxy-2,2′-bipyridine (4D), and K₄[Ru(tctrpy)₂] or k₄[Os(tctrpy)₂] where tctrpy=4,4′,4″-tricarboxy-2,2′:6′,2″-terpyridine (4E);

FIGS. 5A and 5B are schematic representations of the synthesis of regular PNIPAM gels: positively charged gels (5A) and negatively charged gels (5B);

FIG. 6A-6H are schematic representations of structures of chemically modified biopolymer chitosan: Succinyl chitosan (6A), Quateraminated chitosan (6B), Octanoyl chitosan (6C), Caproyl/Myristoyl/Palmitoyl chitosan (6D), Chitosan thioglycolic acid (6E), Conjugated/Thiol containing chitosan (6F), Phosphorylated chitosan (6G), and Carboxy Methyl chitosan (6H);

FIG. 7A shows schematic structures of chemically modified thermo-responsive PNIPAM gels, along with thermosensitive PNIPAM grafted chitosan polymer;

FIGS. 7B and 7C show unmodified negatively charged polymers: Poly-acrylic acid (7B) and alginic acid (7C);

FIGS. 7D-7F show unmodified positively charged polymers: Chitosan (7D), PNIPA-amine polymer (7E), and polyamido amine polymer (7F);

FIGS. 7G-7L show structures of unmodified neutral polymers that can be modified in to positively charged or negatively charged based on chemical modification: Polyvinyl alcohol (7G), hydroxy propyl cellulose (7H), polyethylene glycol (7I), agarose (7J), poly-N-isopropylacryl amide (7K), and (hydroxyethyl)-methacrylate (7L);

FIGS. 8A and 8B show the mechanism of stabilization of polyionic luminescent metal based phosphors in various forms of gels/polymers: Electrostatic attraction that lead to size reduction in the phosphorescent microgels of PNIPAM-co-allylamine (8A) and formation of a chemically-crosslinked phosphorescent hydrogel network of PNIPAM-co-allylamine. The iridescent color of the crystalline product (or lack thereof to attain a colorless hydrogel network) can be controlled by varying the polymer concentration while the phosphorescence color remains the same (8B);

FIGS. 9A and 9B show the formation of thermosensitive/pH sensitive biopolymer based polymeric nanoparticles by taking advantage of anionic/cationic functional moieties of polyionic metal based phosphors: In pH sensitive bio-polymer chitosan (9A), in thermosensitive PNIPAM polymer (9B);

FIG. 10 shows changes in PNIPAM hybrid microgels in presence of polyionic metal based phosphor during gelation demonstrated explicitly inside a positively charged gel system using a polyanionic Au based phosphor;

FIG. 11A shows steady state photoluminescence data for pH and functional group dependent loading of gold phosphor into different PNIPAM microgels;

FIG. 11B shows photoluminescence spectra at room temperature for different forms of phosphorescent hydrogels: (a) microgels; (b) chemically-crosslinked bulk hydrogel; (c) freeze-dried xerogel;

FIGS. 12A and 12B show changes in hydrodynamic radius of positively charged PNIPAM gels in presence of anionic metal based phosphors demonstrating physical interactions and stability: In presence of Au based phosphor (12A), in presence of Pt(II) metal based phosphor (12B);

FIG. 13 shows turbidity vs. wavelength measurements for positively charged PNIPAM microgels after loading metal based phosphors at different temperatures;

FIGS. 14A-14D show the photoluminescence enhancement in hybrid positively charged microgels in presence of polyanionic gold based phosphor at RT with respect to Au phosphor concentration: 3×10⁻⁶M (14A), 5×10⁻⁶M (14B), 1×10⁻⁵M (14A), and 5×10⁻⁵M (14D);

FIGS. 15A-15D show photoluminescence enhancement of Au phosphor vs. microgel concentration: FIGS. 15A and 15B show results for a titration of an aqueous solution of ˜1×10⁻⁶ M Au phosphor (2.50 mL) with 0.1-mL increments of ˜2.0 wt. % PNIPAM-co-allylamine microgel at pH 5.5 and room temperature, FIGS. 15C and 15D show results of a similar titration for a 1×10⁻⁵ M sample of a freshly synthesized Au phosphor (3.00 mL) with 20-4, increments of ˜3.5 wt. % microgel;

FIG. 16 shows the temperature-dependent photoluminescence enhancement in positively charged hybrid microgel upon heating from room temperature (RT) to 37° C., contrasted with quenching for aqueous polyanionic Au based phosphor;

FIG. 17 shows the change in photoluminescence spectra demonstrating rigidochromic shift from polyanionic Au based phosphor in positively charged microgels both in ‘Sol’ and ‘Gel’ forms;

FIGS. 18A-18C demonstrate formation of positively charged polymeric nanoparticles in presence polyanionic Au based phosphor with chitosan biopolymer: Changes in size with of polymeric particles with changes in concentration of phosphor (18A), photoluminescence for the same (18B), and images of particles at room light and on UV exposure (18C);

FIG. 19 demonstrates changes in size with respect to changes in pH from phosphorescent chitosan nanoparticles;

FIG. 20 demonstrates the minimization of usage of toxic chemical crosslinkers in presence of polyanionic Au based phosphor for chitosan nanoparticles;

FIGS. 21A and 21B demonstrate the formation of positively charged polymeric nanoparticles in presence polyanionic Au based phosphor with thermo-sensitive NIPA polymer: Changes in size of polymeric particles with changes in concentration of phosphor (21A) and photoluminescence for the same (21 B);

FIG. 22 shows the thermosensitive behavior of phosphorescent PNIPAM-RCONH₂ polymeric particles with respect to changes in temperature;

FIG. 23 shows ¹H NMR spectra of phosphorescent CSNP in D₂O;

FIG. 24 is the FTIR spectra of chitosan polymer, AuP and Phosphorescent chitosan nanoparticles;

FIGS. 25A and 25B show scanning Electron Microscopy (SEM) images of phosphorescent positively charged chitosan particles;

FIGS. 26A-26D show high resolution transmission electron microscopy (HRTEM) images of phosphorescent positively charged chitosan particles;

FIGS. 27A-27D show HRTEM images of phosphorescent chitosan nanoparticles;

FIG. 28 shows the steady state and lifetime data for polyanionic Au phosphor doped chitosan films/powder (inset shows luminescent chitosan film);

FIGS. 29A and 29B show the photoluminescence spectra for chitosan polymer stabilized anionic Au trimer system (pyrazole based) completely in aqueous medium: At pH 3.0 (29A) and at pH 6.0 (29B);

FIGS. 30A and 30B show the changes in photoluminescence spectra for chitosan polymer stabilized anionic Au trimer system (pyrazole based) before (30A) and after doping Ag+ ions (30B);

FIGS. 31A and 31B show the changes in photoluminescence spectra for chitosan polymer stabilized anionic Au trimer system (pyrazole based) before (31A) and after doping Pb⁺² ions (31B);

FIGS. 32A and 32B show changes in photoluminescence spectra for chitosan polymer stabilized anionic Au trimer system (pyrazole based) before (32A) and after doping Ti⁺³ ions (32B);

FIG. 33 shows the retained film forming ability of biopolymer chitosan even after doping different anionic Au based phosphors;

FIG. 34 shows the changes in emission colors or tuning of emission colors in positively charged chitosan biopolymer-stabilized anionic cyclic Au trimer systems either by varying pH or heavy metal ion entrapped;

FIG. 35 shows the temperature dependent hydrodynamic radius changes in strong gelation samples vs. plain microgel (Inset shows complete changes beyond gelation point);

FIGS. 36A and 36B show the formation of phosphorescent PNIPAM-RCONH2-AuP polyelectrolyte complex nanoparticles and PL spectra for the same: Rh changes with respective to AuP concentration (36A) and PL spectra for the same (36B);

FIGS. 37A and 37B show ³¹P{¹H} NMR spectra in D₂O for the Na₈[Au(TPPTS)₃] sample prepared in the present invention (37A) vs. the free TPPTS ligand (37B). Note the main broad peaks at 45.04 & 42.14 ppm for the Na₈[Au(TPPTS)₃] complex concomitant with low intensities of peaks due to free ligand, and the low intensity of the TPPTS oxide peak at 34.2 ppm in the spectrum of the free ligand (purchased as 95% purity);

FIG. 38 shows the steady-state photoluminescence (in the solid state and aqueous solution) and UVN is electronic absorption spectra (aqueous solution) of Na₈[Au(TPPTS)3] samples prepared in the present invention;

FIGS. 39A and 39B show the ¹H NMR spectra in D₂O for the Na₈[Au(TPPTS)₃] sample prepared in the present invention (38A) vs. the free TPPTS ligand (38B);

FIGS. 40A and 40B show the time-resolved photoluminescence decay of Na₈[Au(TPPTS)₃] aqueous solution prepared in the present invention. The data were acquired using either: N₂ laser pulsed excitation (337.1 nm) and a stratosphere nanosecond detector (40A) or xenon flashlamp excitation (337 nm) and a gated microsecond detector (40B). The decay analysis included deconvolution of the pulsed excitation signal using the Felix software for the PTI instrument; and

FIGS. 41A and 41B show IR spectra for KBR pellets of the Nag[Au(TPPTS)₃] sample prepared in the present invention (41A) vs. the free TPPTS ligand (41B).

### DETAILED DESCRIPTION OF THE INVENTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not delimit the invention, except as outlined in the claims.

As used herein the term ‘transition metal” should be understood to include elements 21 through 30, 39 through 48, 57 through 80, and 89 through 103 of the Periodic Table. In chemical terms, these are elements having a partially filled inner shell of electrons. Alternatively, these are elements that are in the d-block and f-block of the Periodic Chart of the Elements; that exhibit a variety of oxidation states; and that can form numerous complex ions. As used herein, the phrase “d-block” includes those elements that have electrons filling the 3 d, 4 d, 5 d, and 6 d orbitals surrounding the nucleus of the element. As used herein, the phrase “f-block” refers to those elements that have electrons filling the 4 f and 5 f orbitals surrounding the nucleus of the element, including the lanthanides and the actinides. Non-limiting examples of preferred transition metals include cobalt, copper, nickel, zinc, vanadium, chromium, platinum, gold, silver, tungsten, and molybdenum.

The term “phosphorescent system” as used herein refers to a system which when exposed to a stimulus, for example an ultraviolet radiation, absorbs such light or component and continues to emit visible light after the stimulus is removed.

As used herein, the term “polyanionic” refers to a chemical entity, for example, an ionically charged species, such as an ionically charged polymeric material, which includes more than one discrete anionic charge, that is multiple discrete anionic charges. The term “polycationic” as used herein refers to agents that in their chemical structure comprise a plurality of discrete positive charges.

The term “hydrogel” as used herein in the specification and claims refers to a polymeric material which exhibits the ability to swell in water and to retain a significant portion of water within its structure without dissolution. “Hydrogels” are typically three dimensional macromolecular networks in water formed from a cross-linked polymer. The term “colloid” as used herein refers to the minute particles suspended in the continuous medium, said particles having a nanometer-scale particle size. The particles in a “colloid” have a particle size of generally less than 10 microns, or the particles have at least one dimension between about 1 and about 1000 nm. As used herein the term “microgel” is used to indicate a three-dimensional hydrogel particle that is 0.1-10 μm in diameter.

The term “microparticle” as used herein, refers to a particle of about 10 nm to about 150 μm in diameter, more preferably about 200 nm to about 30 μm in diameter, and most preferably about 500 nm to about 10 μm in diameter.

The term “nanoparticle” as used herein in various embodiments will be understood by one of ordinary skill to encompass all materials with small size and quantum size confinement, generally the size is less than 100 nm.

The term “chemical crosslinking agent” as used herein and in the claims refers to any chemical agent capable of covalently binding to polymers or biomaterials to form a crosslinked network.

The term “opalescent” refers to a type of dichroism seen in highly dispersed systems with little opacity. The material appears yellowish-red in transmitted light and blue in the scattered light perpendicular to the transmitted light.

The terms “gelation temperature” or “gel/sol temperature” as used herein refers to the temperature at which a solution transitions to become a gel.

As used herein the term “carcinogen” refers to a compound (e.g., a chemical, a protein, a molecule) which causes a cell to demonstrate transformation characteristics which can include any properties associated with tumor or cancer cells. In particular, such characteristics can include formation of foci, anchorage independence, loss of growth factor or serum requirements, change in cell morphology, ability to form tumors when injected into suitable animal hosts, and/or immortalization of the cell. The presence of one or more of such transformation characteristics is indicative that the compound is “carcinogenic”.

The term “photoluminescent” as used in the present invention refers to any material that absorbs and stores energy when illuminated by visible or invisible (e.g. ultraviolet or infrared) light and that later reradiates the stored energy as visible or invisible light. “Photoluminescent” materials are particularly useful in applications in which there are no sources of natural light or electrical power during emergency situations. They are used in commercial, industrial, and transportation environments for identifying emergency escape routes and danger areas, (such as stairs and doors) and for marking equipment, pipes, tools, fire equipment (such as fire hoses and extinguishers), and clothing for work or accident prevention.

The term, “in vitro” is defined herein as being an artificial environment outside the living organism (e.g., a petri dish or test tube). The term “in vivo” as used herein refers to processes that occur in a living organism.

The terms “administration of” or “administering a” compound refers to providing a compound of the invention to the individual in need of treatment in a form that can be introduced into that individual's body in a therapeutically useful form and therapeutically useful amount, including, but not limited to: oral dosage forms, such as tablets, capsules, syrups, suspensions, and the like; injectable dosage forms, such as IV, IM, or IP, and the like; transdermal dosage forms, including creams, jellies, powders, or patches; buccal dosage forms; inhalation powders, sprays, suspensions, and the like; and rectal suppositories.

The terms “effective amount” or “therapeutically effective amount” refers to the amount of the subject compound that will elicit the biological or medical response of a tissue, system, animal or human that is being sought by the researcher, veterinarian, medical doctor or other clinician.

As used herein, the term “treatment” or “treating” indicates any administration of a compound of the present invention and includes (1) inhibiting the disease in an animal that is experiencing or displaying the pathology or symptomatology of the diseased (i.e., arresting further development of the pathology and/or symptomatology), or (2) ameliorating the disease in an animal that is experiencing or displaying the pathology or symptomatology of the diseased (i.e., reversing the pathology and/or symptomatology).

The present invention describes multiple synthetic procedures of stimuli-responsive phosphorescent hydrogel microspheres by incorporating a known water-soluble phosphorescent Au(I) complex, Na8[Au(TPPTS)3], TPPTS=tris(3,3′,3″-trisulfonatophenyl)phosphine, into the polymer network of poly-N-isopropylacrylamide (PNIPAM). Further the present invention provides a novel method for stabilizing and enhancing photoluminescence properties of phosphorescent complexes of transition metals in general and noble transition metals (Gold Silver, Copper, Platinum, Ruthenium, and Osmium) in particular in a wide variety of soft gels and polymers which is particularly significant for aqueous or non-aqueous compatible systems as described herein.

There are a few examples in the literature explaining the effect of PNIPAM or other polymer-based systems on the luminescent properties of lanthanide complexes. For example, Huang and coworkers1 detail the interactions of the lanthanide ion Tb(III) with PNIPAM-co-styrene microspheres. McCoy and coworkers2 reported the incorporation of Eu(III)-based complexes into methyl methacrylate: hydroxyethyl methacrylate (MMA:HEMA) hydrogels. Yan et al. 3, showed that the formation of PNIPAM polymer chain by incorporating a Tb(III) complex in the solid state, lead to emission enhancement of Tb(III) in PNIPAM/Tb(III) systems due to effective intramolecular energy transfer from the PNIPAM polymer chain to the Tb(III) centers. However, a noble transition metal based phosphor has not been stabilized in such a wide variety of soft gel systems with complete retention and improvement in phosphorescent properties as in the present invention. Though photoluminescence enhancement is known previously either due to scattering from noble metal nanoparticles or in presence of micelles, two order magnitudes of enhancement as shown herein has not been reported or discussed elsewhere.

Polymer solutions with thermoreversible gelation are highly sought after because it may be used as an injectable scaffold for delivering medicine or encapsulating cells 4. A typical system is gelatin solution that undergoes normal sol-gel transition without chemical cross-linking 5-6. Other polymeric solutions have an inverse (that is, gelation upon heating) thermoreversible gelation including triblock copolymers (Pluronic or Poloxamer) or degradable triblock copolymers 7-8. The present inventors recently report an inverse thermoreversible gelation from PNIPAM-PAAc IPN microgels 9 and Gan and co-workers have reported in-situ gelation using (P(NIPAM-HEMA)) based microspheres as building blocks in presence of CaCl2 and used them as injectable scaffolds in cell encapsulation studies. In all these cases, though physical gelation/aggregation/flocculation behavior in PNIPAM and other block polymers is attributed to physical interactions between oppositely charged polymer systems or due to presence of simple electrolytes like NaCl or NaNO3 or CaCl2 at high concentrations (˜0.08M), but nobody has ever used a phosphorescent molecular system as luminescent gelating agent as reported herein by the present inventors.

Formation of polyelectrolyte complex nano/micro particles is known from chitosan or other natural or pH/thermosensitive polymers in presence of oppositely charged polymers or anionic systems like tripolyphosphate or dextran sulfate. But luminescence is inducted into these polymeric particles mostly by doping with an external luminophore. Only a few examples of phosphorescent materials with crosslinking capabilities are known, mainly/only from Ruthenium, Rhenium, or Iridium transition metal complexes acting as crosslinking agents or molecular probes 10-11. Thus, the present invention presents a novel application using mainly transition metal based phosphors. The transition noble metal based phosphors presented here have dual roles: (i) physical crosslinkers that result in either gelation of microgel or hydrogel particles above their LCST or crosslinking of linear polymer chains into microgels, microparticles, or nanoparticles; and (ii) light emitting centers.

Formation of gold (I) based triazole/pyrazole for sensing applications is already know in organic solvents but stabilization in aqueous medium is very much unknown due to poor solubility and stability issues of these trimer systems. And also chitosan polymer property to entrap different metal ions from water due to chelation property of chitosan polymer is well studied. The present invention takes advantage of the same chelation property of chitosan but by doping a phosphorescent transition metal complex, wherein hazardous heavy metal ion sensing is demonstrated through dramatic changes in luminescence colors and/or intensities upon interaction of the sensors herein with a variety of hazardous heavy metal ions. Such novel soft material phosphors have significant applications in sensing and bioimaging.

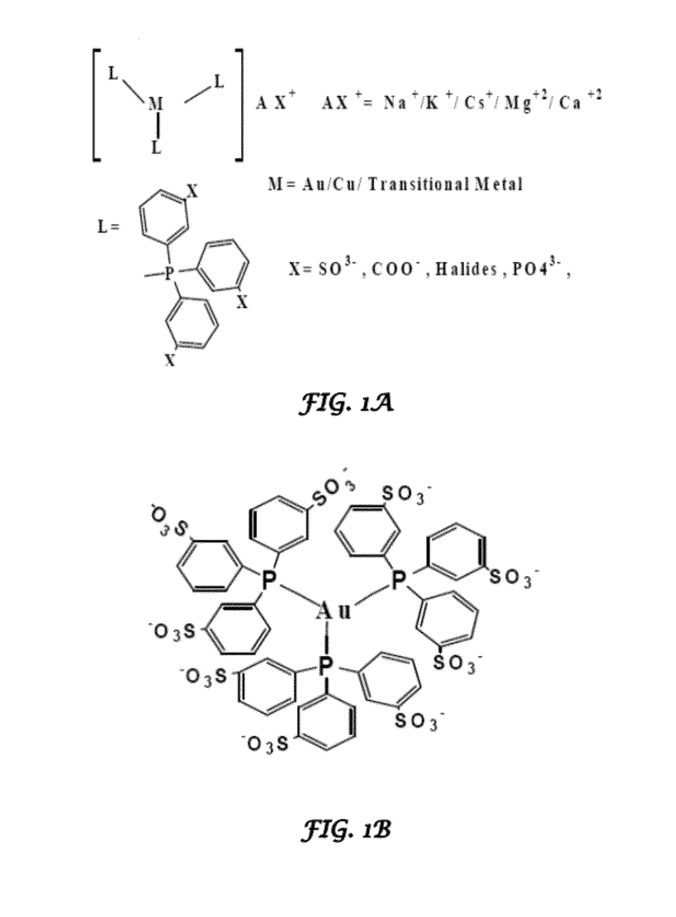

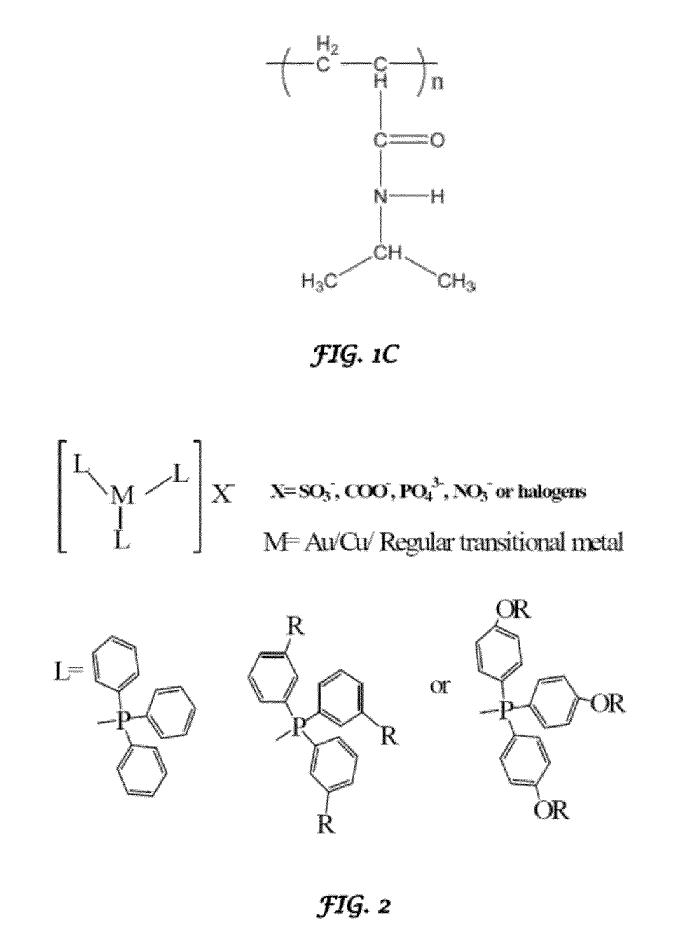

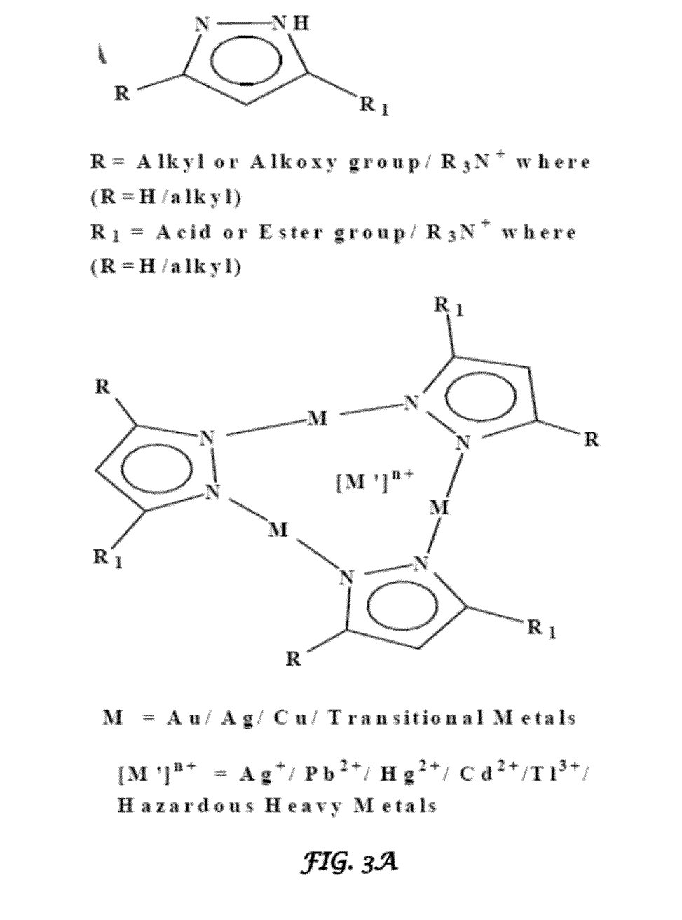

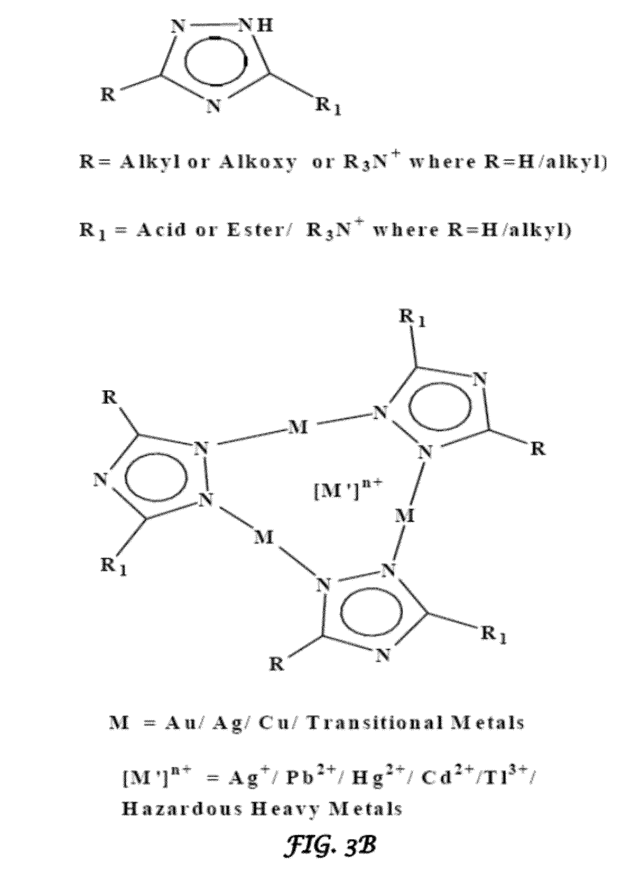

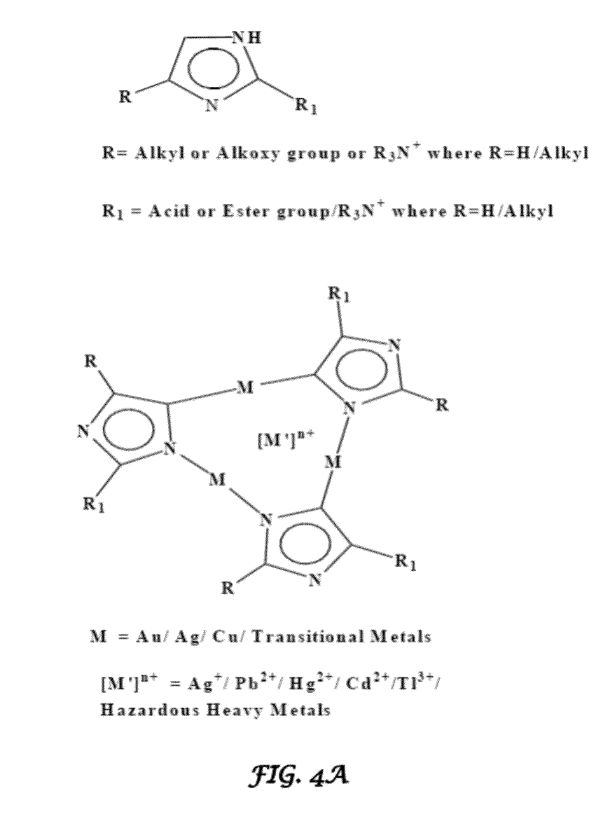

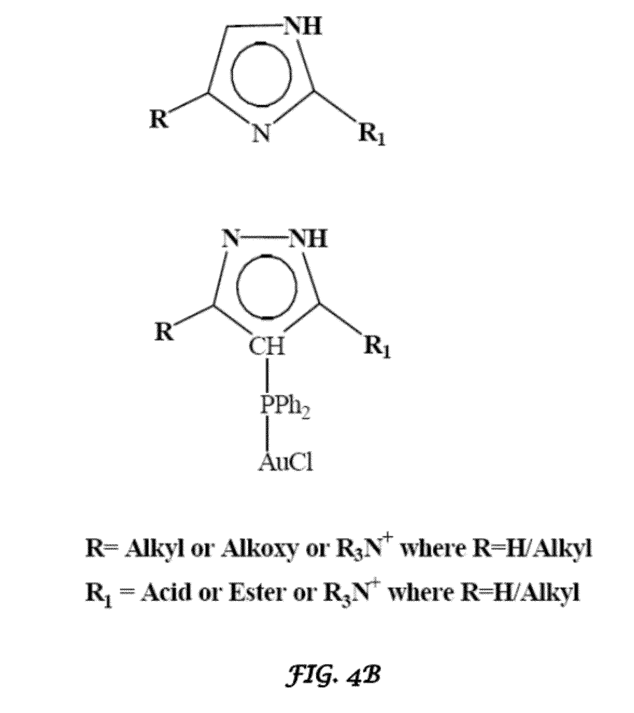

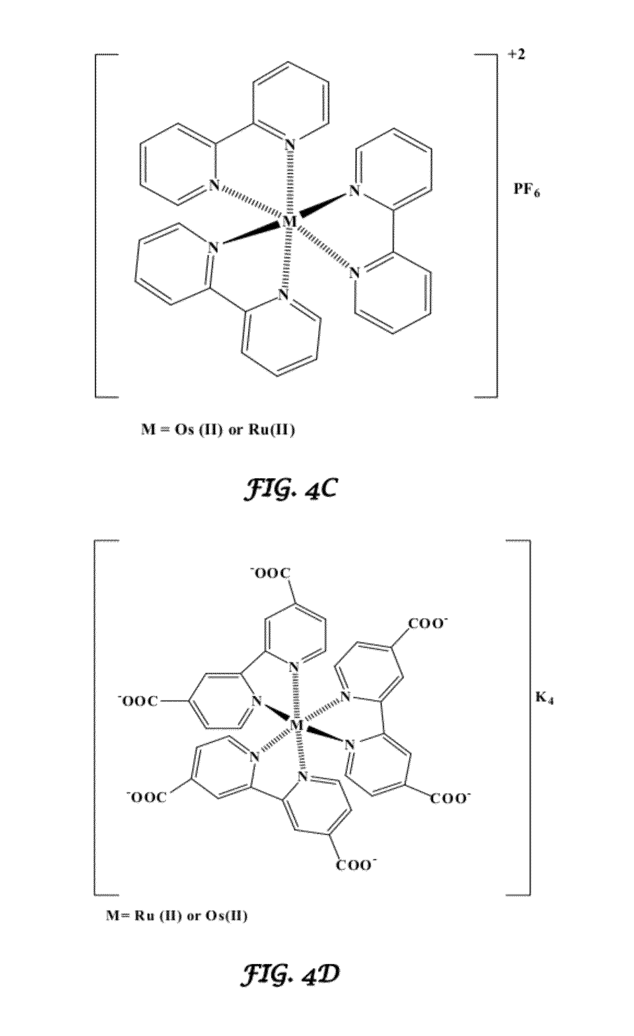

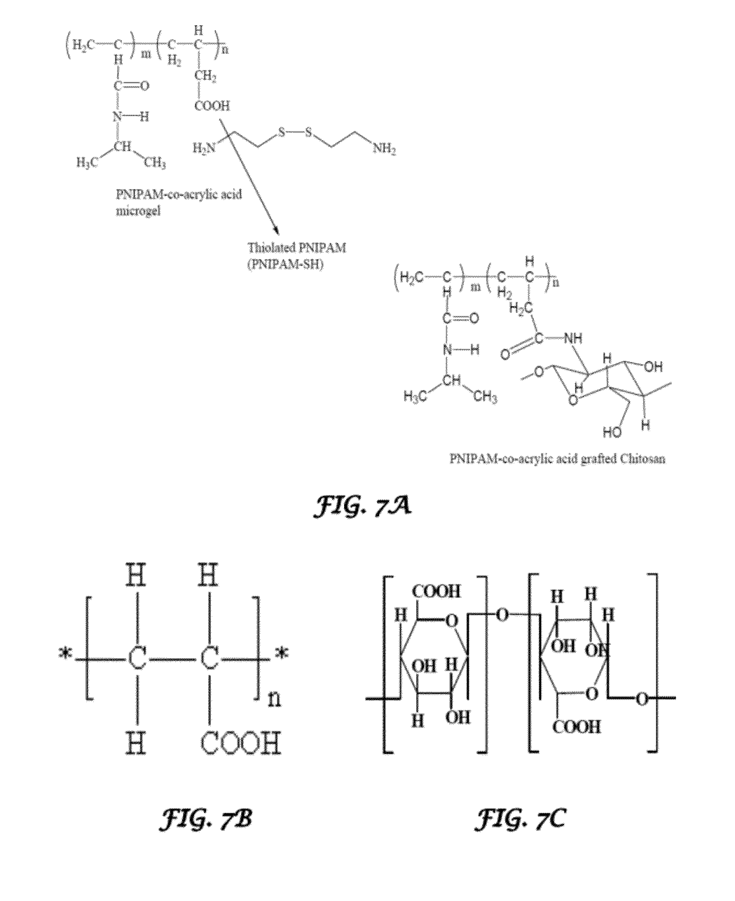

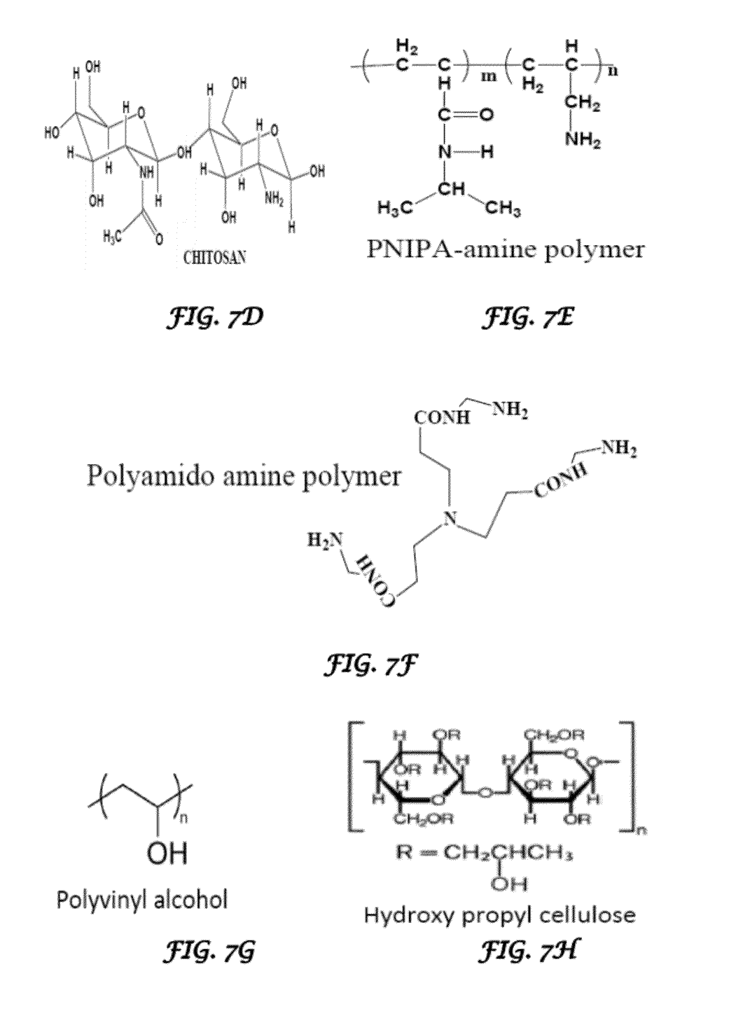

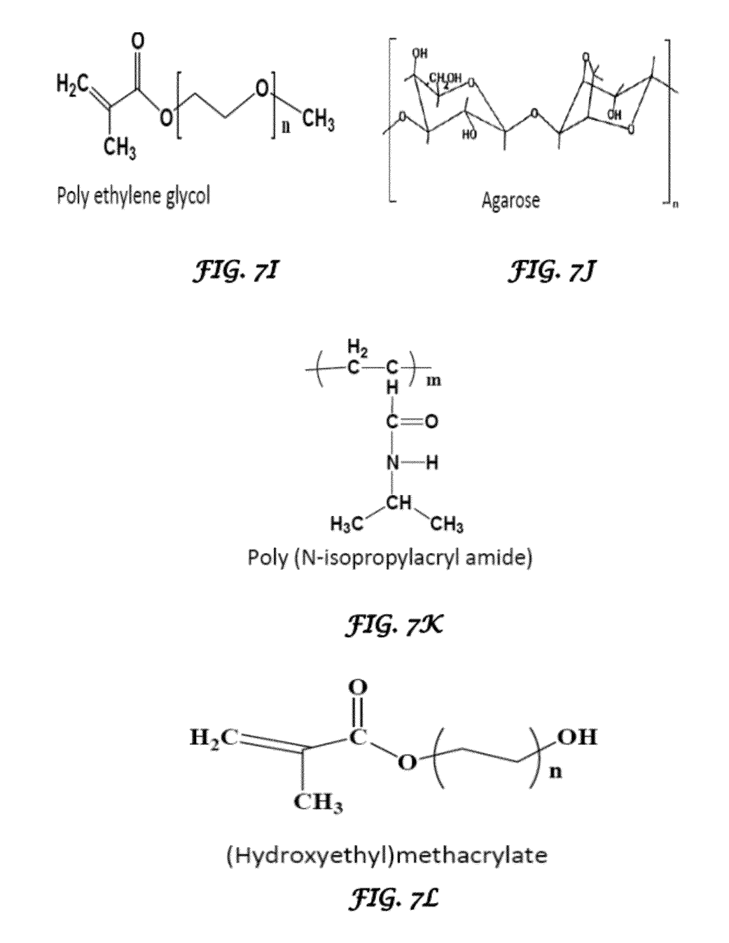

FIGS. 1A and 1B show the general schematic structures of polyanionic transition metal based phosphors described in the present invention. FIG. 2 shows general schematic structures of polycationic transition metal based phosphors. FIGS. 3A, 3B, 4A and 4B show the structures of cyclic transition metal based trimers synthesized using different azole based ligands, whereas FIGS. 4C, 4D and 4E show Ru(II) and Os(II) based phosphors that are stabilized in different biopolymers and gels. FIGS. 5A, 5B, 6A-6H, and 7A-7L show the structures of chemically un-modified or modified polyionic biocompatible/stimulus sensitive (including thermo-responsive and pH sensitive) polymers and gels that are used for stabilizing different transition metal based polyionic phosphor systems proposed herein. The unmodified biocompatible/stimulus sensitive polyionic charged polymers in the present invention include poly acrylic acid (PAA), polyvinyl alcohol (PVA), alginic acid, PEG, agarose, hydroxy propyl cellulose, polyamido amine, hydroxyethyl methacrylate.

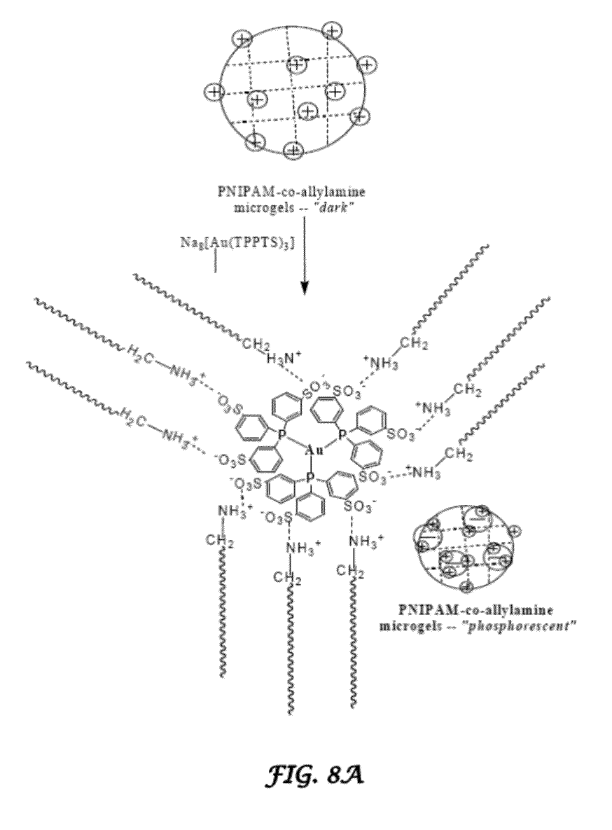

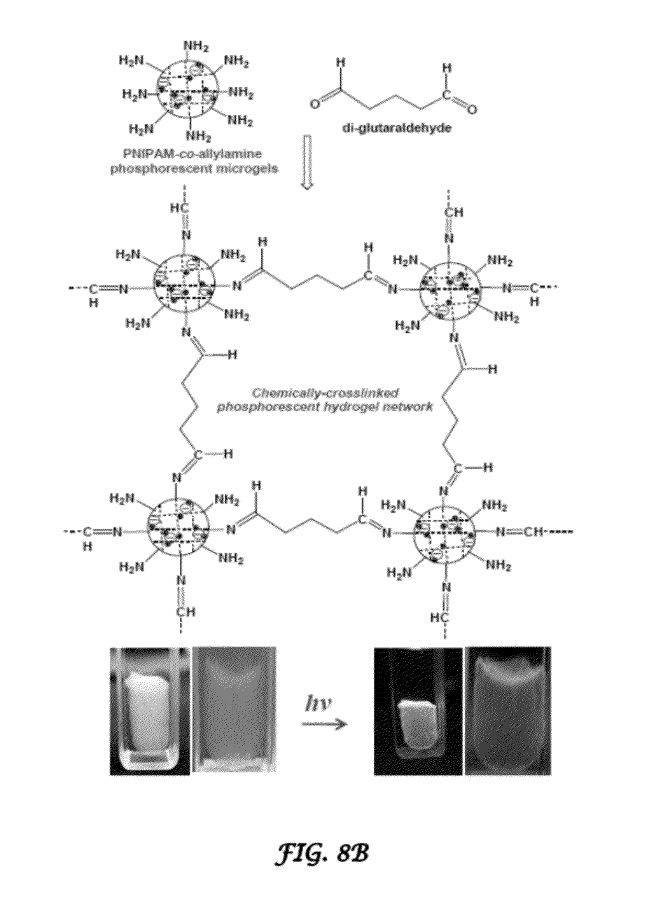

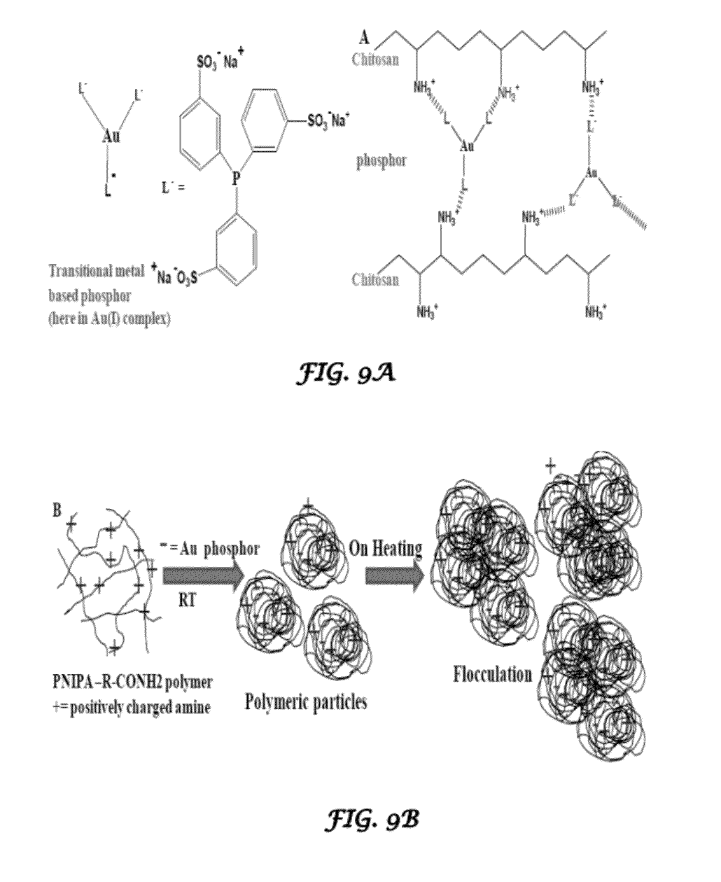

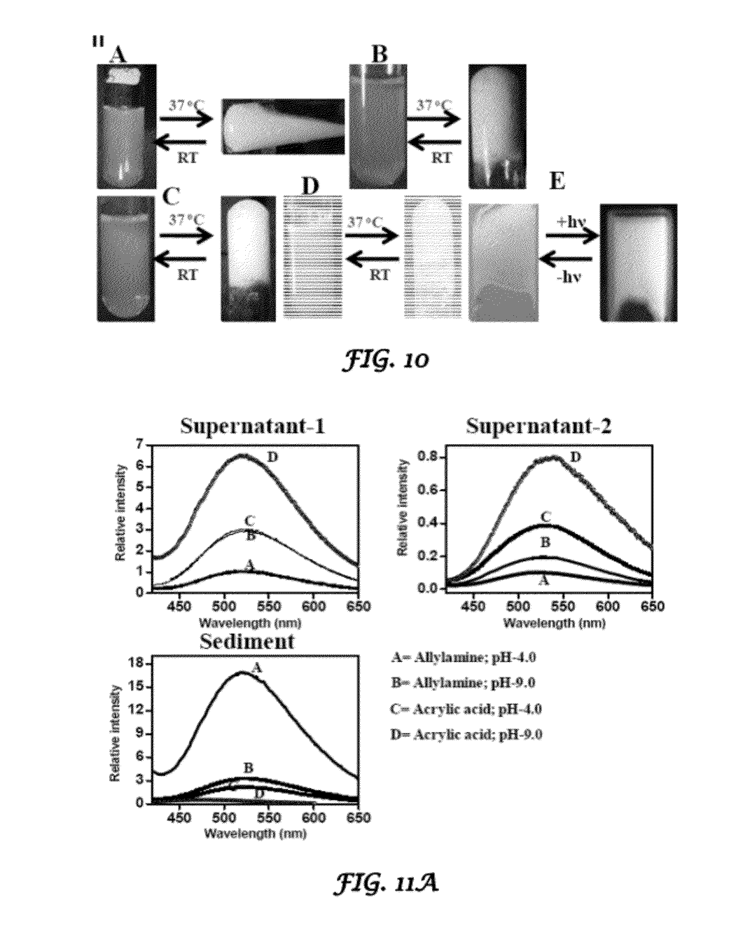

FIGS. 8A and 8B depict the stabilization of polyanionic metal based phosphors inside positively charged soft gels/polymers in general. FIGS. 9A and 9B depict the schematic formation of polymeric nanoparticles where the transition metal based phosphor is acting as physical crosslinker. FIG. 10 depicts physical changes of positively charged phosphorescent microgels in presence of polyanionic gold based phosphor during gelation.

The characterization of the phosphorescent hybrid gels was done by measuring their electron microscopy, zeta potential, and photoluminescence, light scattering, UVN is absorption, FTIR and NMR spectra.

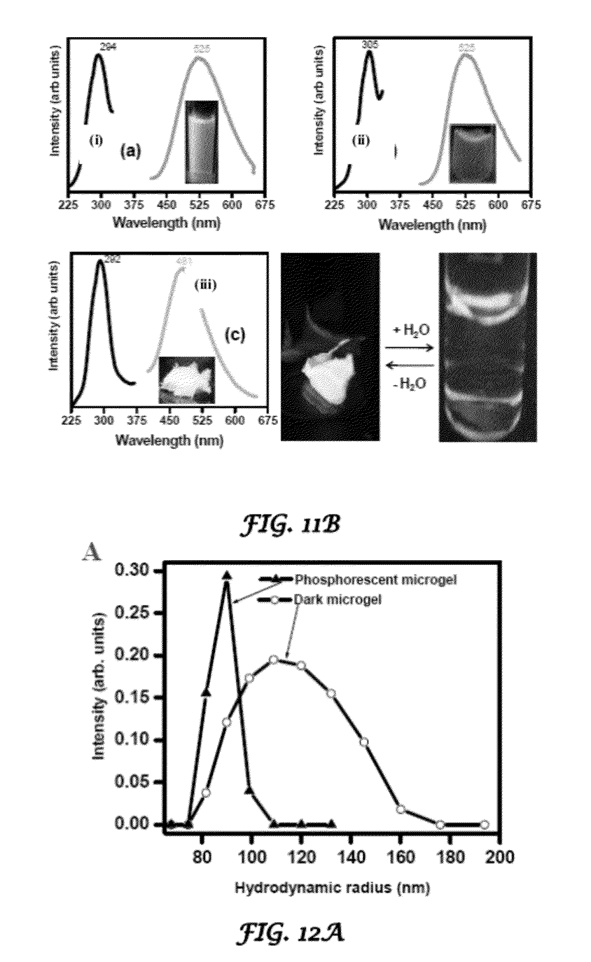

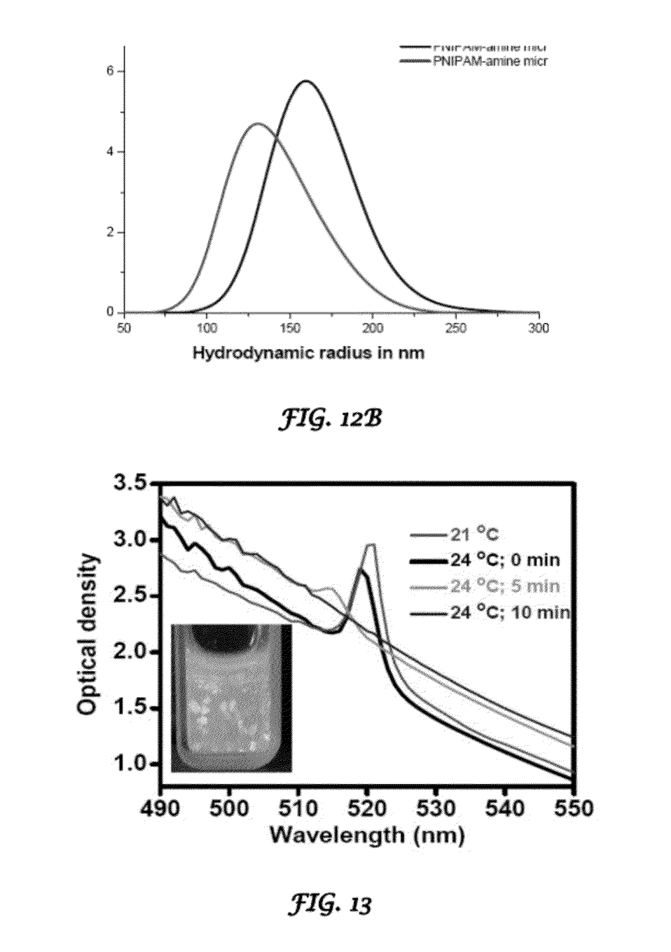

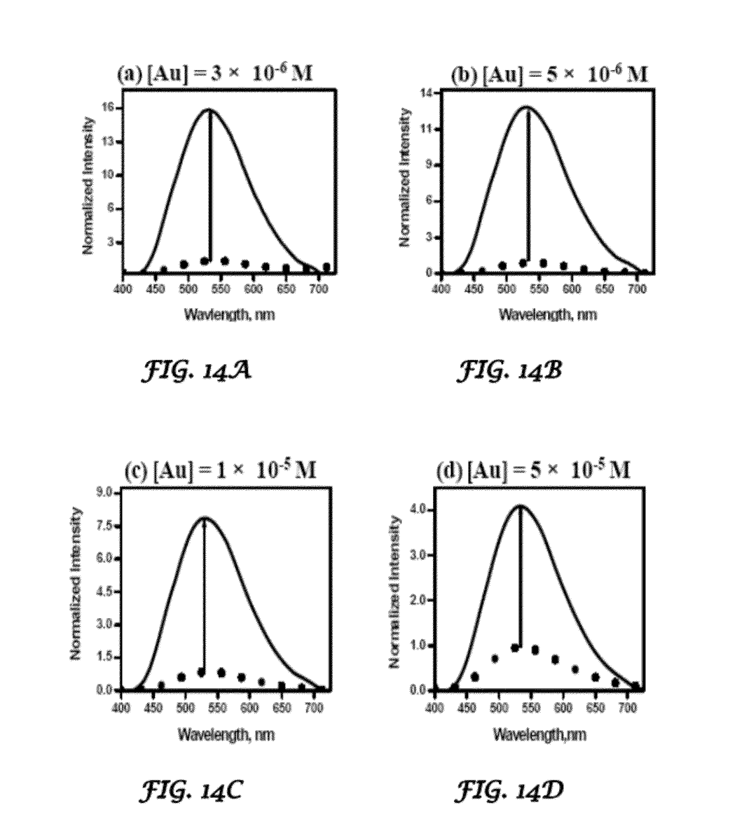

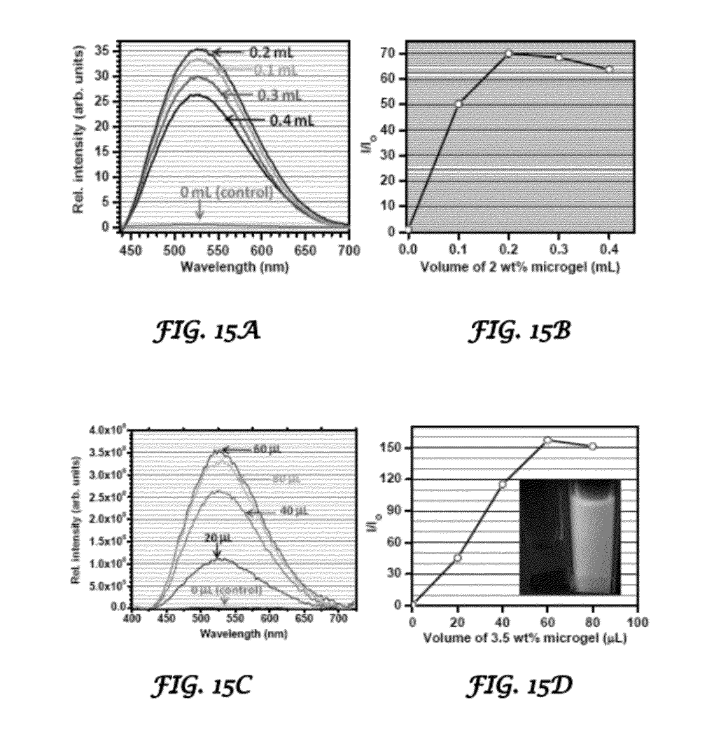

FIGS. 11A and 11B show photoluminescence spectra from different forms of hybrid microgels in presence of transition metal based phosphor and how pH and functional group dictate a loading efficiency of the same in two different PNIPAM gels. FIGS. 12A, 12B, and 13 show changes in hydrodynamic radius and turbidity of microgels before and after loading the transition anionic metal based phosphor in two positively charged PNIPAM gels, which signifies the interactions between gels and phosphor. FIGS. 14A-14D, 15A-15D, and 16 demonstrate an unusual more than order magnitude photoluminescence enhancement from transition metal based phosphor in presence of PNIPAM microgels in complete aqueous medium by varying concentration of metal phosphor, microgel concentration, pH, and temperature.

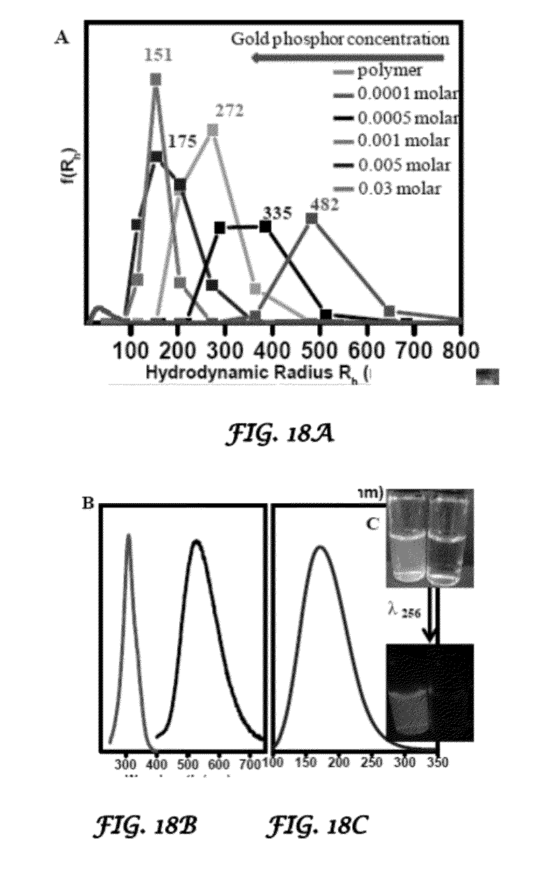

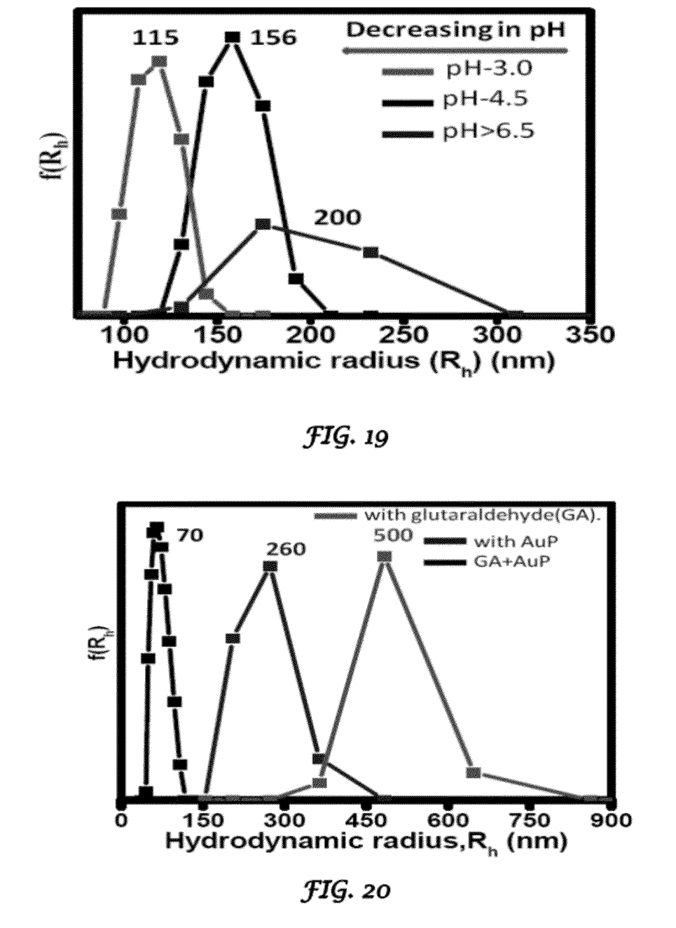

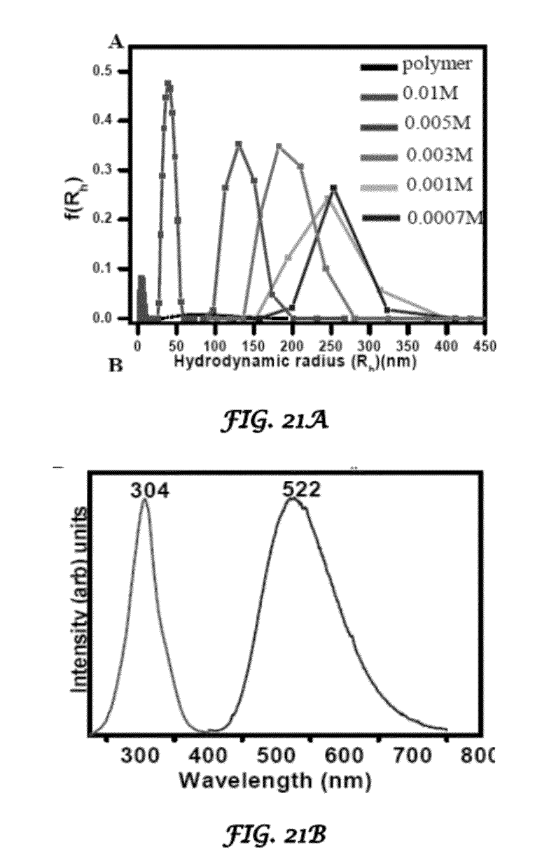

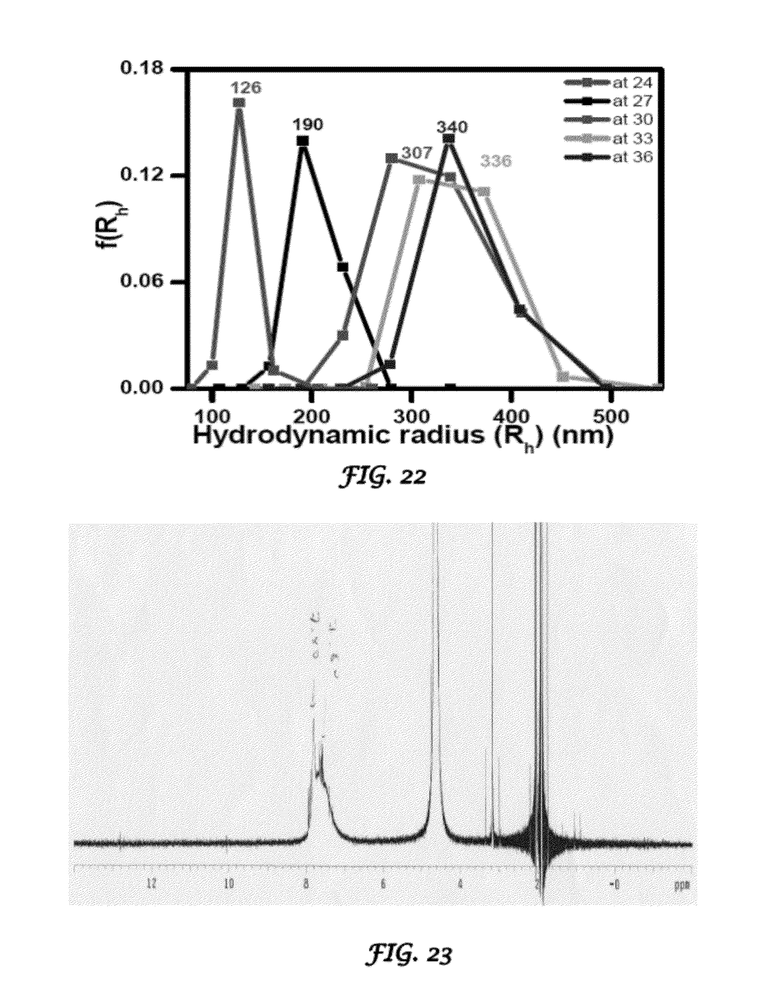

FIG. 17 shows a change in PL emission from transition metal based phosphor upon gelation and changes in hydrodynamic radius of microgels at various concentrations of phosphor on gelation. FIGS. 18A-18C and 19 show PL emission from chitosan based nanoparticles formed from physical crosslinking of metal based phosphor and effect of phosphor concentration and pH on size and distribution of chitosan polymeric particles. The chemical crosslinking capability of glutaraldehyde a well known chemical crosslinking agent's capability is enhanced in presence of our metal based phosphor which results in lowering of toxic chemical crosslinker's (FIG. 20) usage for formation of similar size polymeric nanoparticles. FIGS. 21A, 21B, and 22 demonstrate physical crosslinking capability of the metal based phosphor presented herein in presence of another temperature sensitive polymer PNIPAM-RCONH2. FIGS. 23 and 24 characterize formation of polymeric nanoparticles from FTIR and NMR data.

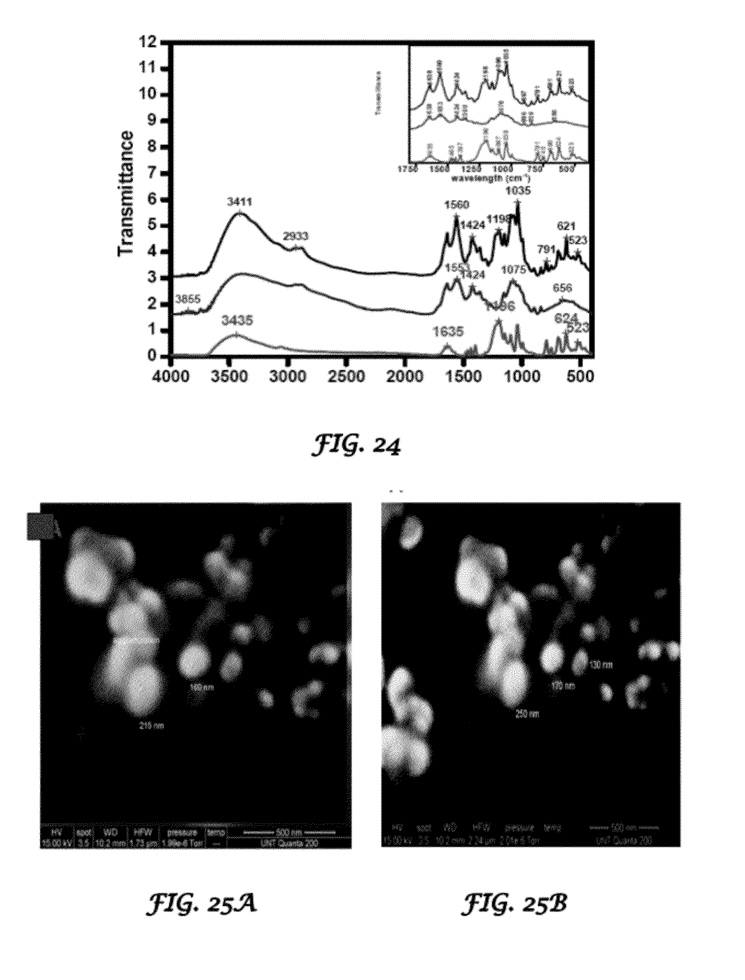

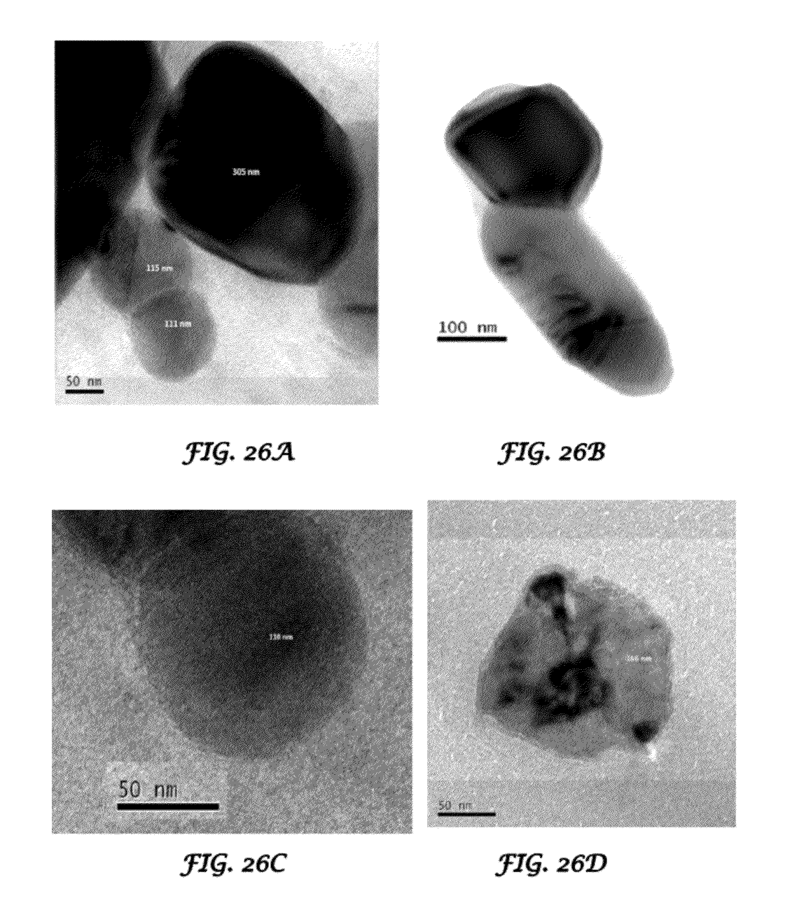

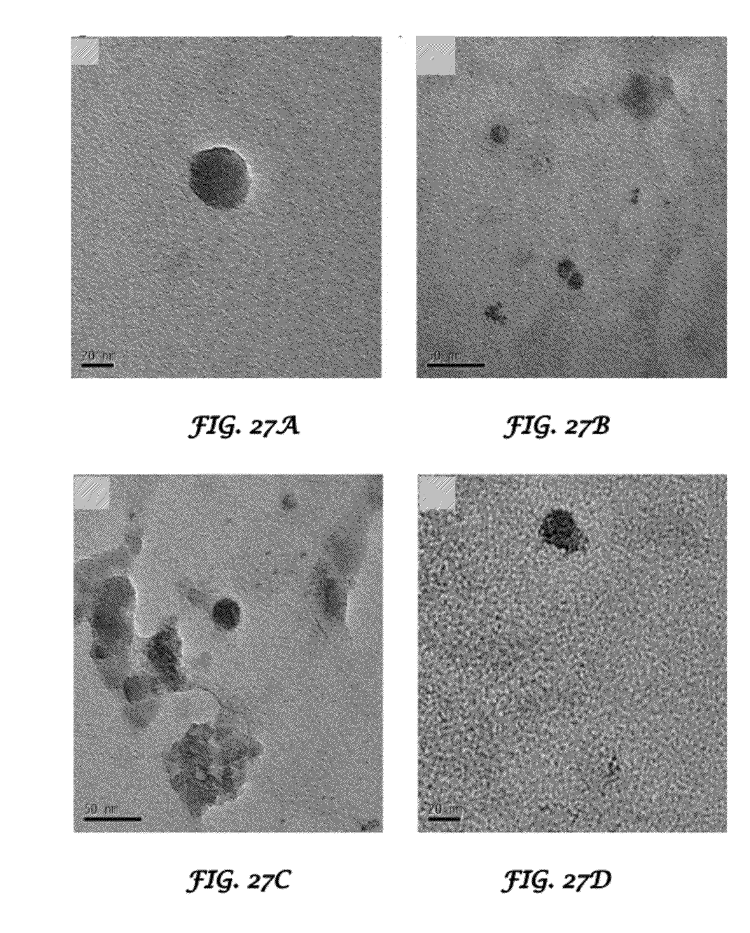

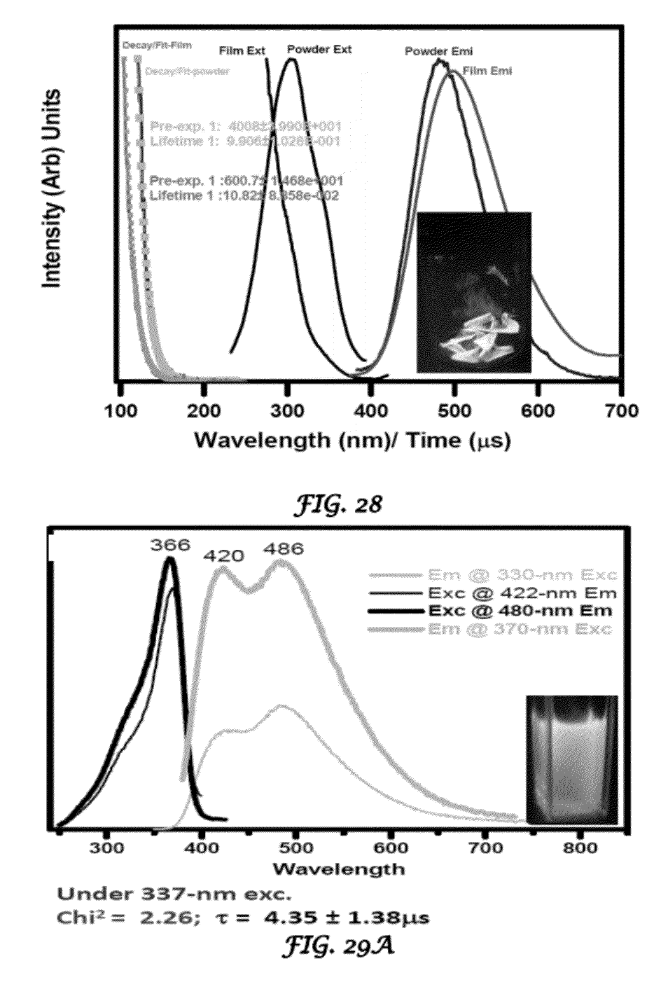

FIGS. 25A and 25B are scanning electron microscopy (FE-SEM) images showing clearly the formation of positively charged polymeric nanoparticles using a polyanionic metal based phosphor. FIGS. 26A-26D and FIGS. 27A-27D demonstrate the tuning of size with wide sizes ranging from 300 nm to 20 nm for the positively charged polymeric nanoparticles just by varying concentration of polyanionic metal based phosphor. FIG. 28 demonstrates retained film forming ability of chitosan after being doped with anionic transition metal based gold phosphor.

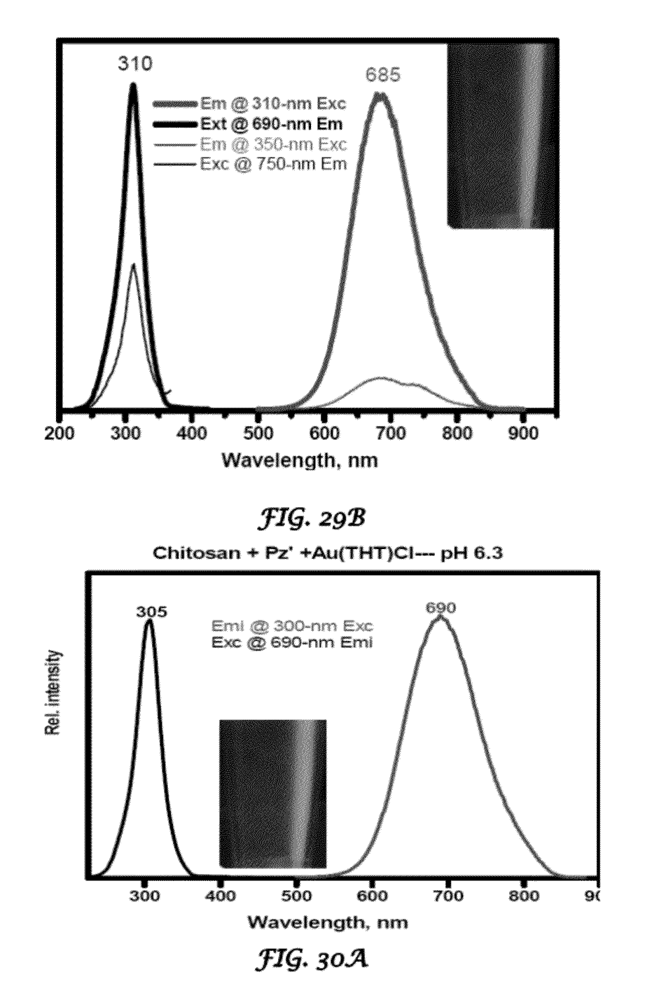

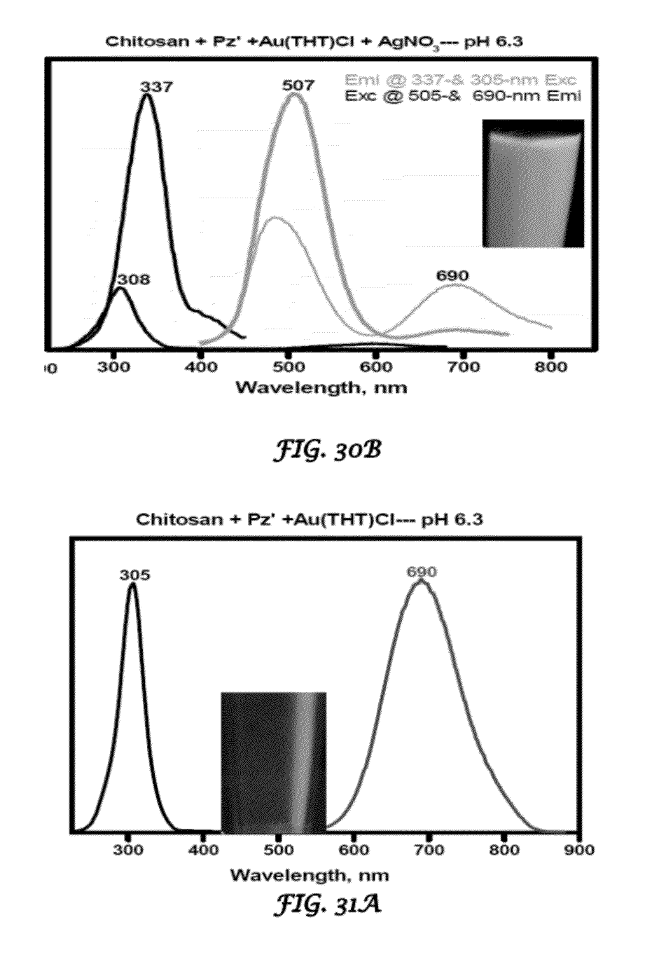

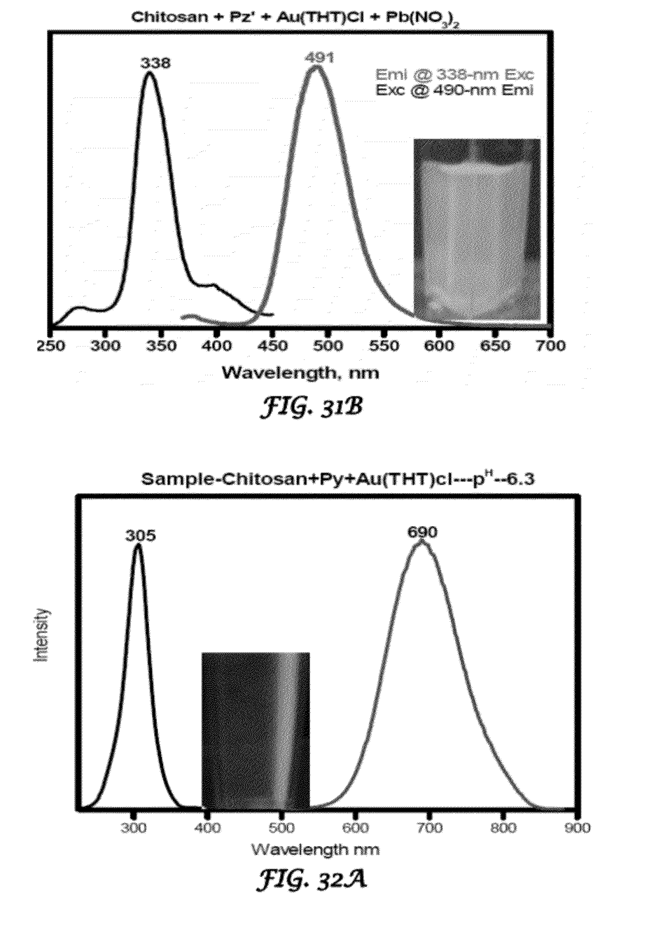

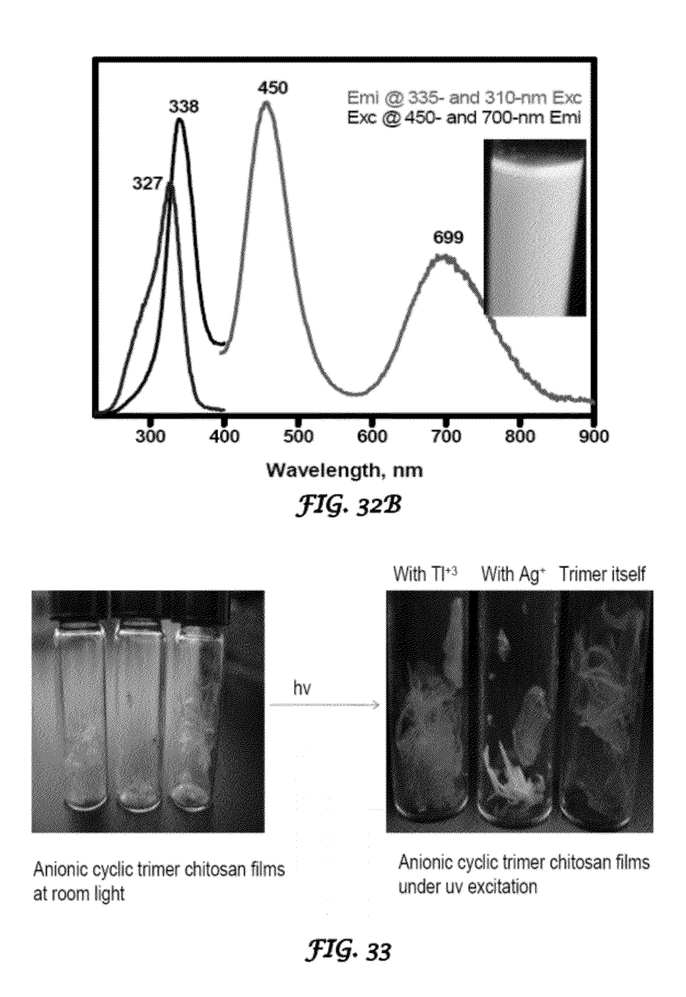

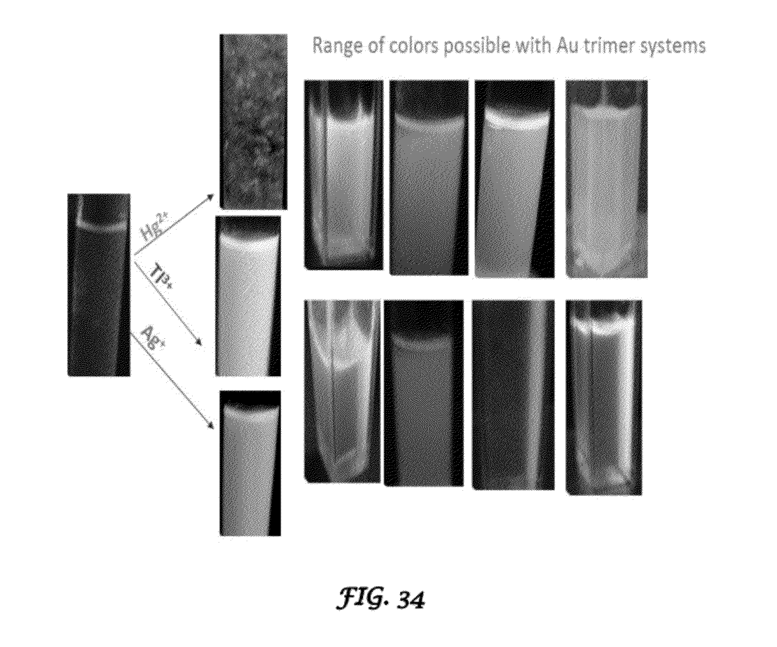

FIGS. 29A and 29B show PL spectra for anionic cyclic Au based trimer system (pyrazole based) stabilized completely in aqueous medium in presence of positively charged chitosan biopolymer under both acidic and neutral pH. FIGS. 30A, 30B, 31A, 31B, 32A, and 32B demonstrate detection and extraction of toxic heavy-metal ions (Ag+/Tl+3/Pb+2) using chitosan stabilized anionic cyclic trimers in aqueous medium by a clear change in emission colors before and after entrapment of different metal ions. FIG. 33 shows different anionic Au(I) cyclic trimer systems doped in chitosan films both under day light and exposure to UV light. FIG. 34 specifically shows how the emission in these Au based anionic cyclic trimers can be tuned after stabilizing in positively charged biopolymer system either by changing in pH or by changing the heavy metal entrapped.

Hydrogels are composed of hydrophilic organic polymer networks crosslinked physically or chemically 12. Some hydrogels exhibit stimulus-sensitive behavior that leads to undergoing conformational changes in response to various external variables such as pH, 13 temperature, 14-17 electric, 18-19 magnetic 20-21 and/or optical stimuli 22-25. Such materials have been called “smart” or “intelligent” hydrogels 26-27 because the aforementioned behavior allows them to be useful for various biomedical applications such as site-specific and controlled drug delivery, 28-38 tunable optics and bio-sensors, 39-40 encapsulation of cells, 41 molecular imaging, 42 immobilization of enzymes and cells, 43 protein assays, 44 separation and waste water remediation, 45-46 and in vitro tissue formation 47-49. Poly-N-isopropylacrylamide (PNIPAM) represents the most extensively-studied stimulus-sensitive polymer hydrogel material 50. Studies on hydrogels already covered a broad domain in materials science; little attention has been paid, however, to hydrogels as hosts for versatile molecular luminophores. Though studies of ligand-lanthanide luminescent molecules in connection with hydrogels as host matrixes are known, 1-3 similar studies using a transition metal-based luminescent complex are yet unexplored, perhaps due to limited pursuit of water-soluble and -stable systems that exhibit sufficiently bright phosphorescence at physiological conditions. Given the quantum leap that phosphorescent transition metal complexes have caused in photonic applications such as organic light-emitting diodes, 51-52 it is important to pursue applications that take advantage of their remarkable photophysical properties by seeking strategies that enable their introduction to biocompatible media such as hydrogels. The present invention fills this gap by introducing the water-soluble phosphorescent transition metal compound Na8[Au(TPPTS)3] into the polymer network of PNIPAM (FIGS. 1B and 1C).

Phosphorescent hydrogels can enable studies of diffusional processes, phase transformations of entangled biopolymers, and environmental stimuli 53. In their swollen state, hydrogels are usually transparent and colorless while in their collapsed state they become turbid. Advances in instrumentation technology have rendered luminescence spectroscopy a very versatile and powerful yet readily accessible tool for the characterization of polymers and hydrogel systems. In order to track the gel swelling behavior or to monitor the location of gel particles in cells, fluorescent dyes or quantum dots are typically used so that the hydrogels are distinguished from the surrounding biological environment 54-58. In this process, fluorescent labels have some disadvantages that include short nanosecond lifetimes that may coincide with any biological background emission (auto-fluorescence), self-quenching effects, high photo-bleaching, and weak fluorescence intensity at physiological pH 59-64. Some of these disadvantages are predicted to be overcome by different emission enhancement mechanisms such as the enhancement in presence of either plasmonic colloidal metal nanoparticles or a micelle medium. Though there is considerable literature work on explaining the mechanism of metal colloid-based enhancement with different fluorophores, little is known about the exact mechanism of fluorescence enhancement mechanism in presence of micelles. Fluorescence enhancement in presence of a micelle medium is predicted to result from a combination of various factors that include enhanced stability or solubility of the fluorophore and/or decrease in non-radiative decay of the fluorophore in presence of micelle medium; however, a definitive mechanism about fluorescence or phosphorescence enhancement in presence of micelle or scattering polymeric colloidal medium is not well-documented.

The present invention demonstrates that the phosphorescence of a three-coordinate gold(I) phosphor can be enhanced by more than an order of magnitude compared to the situation in a plain aqueous medium at different temperatures. While Raman scattering enhancement is well-documented for detection of small molecules at very low concentrations, the invention presented herein paves the way to a wider scope of research by the simpler and more widely accessible method of steady-state photoluminescence spectroscopy to enable scattering-stimulated enhanced detection of luminescent molecules at very low concentrations. Thus, the inventors demonstrate the loading of a highly-stable water-soluble polyanionic three-coordinate gold(I) complex, [Au(TPPTS)₃]⁸⁻, into stimulus-responsive PNIPAM hydrogel colloidal particles in aqueous medium without sacrificing the environmental sensitivity of the hydrogel while simultaneously enhancing the Au-centered phosphorescence. The selection of three-coordinate Au(I) complexes as phosphors for this is based on the fact that some of these complexes can maintain their luminescence even in aqueous solution⁶⁵⁻⁶⁸.

Materials: N-isopropyl acrylamide=“NIPA monomer” and N,N′-methylene-bis(acrylamide)=“BIS”, SDS, allylamine, acrylic acid monomers are purchased from Polysciences Inc. The Na₉TPPTS, CS₉TPPTS, unmodified Pyrazole, Triazole, Imidazole, Cu₂O, AgNO₃, potassium thiocyanate, ammonium thiocyanate, tetrabutylammonium thiocyanate, tetrabutylammonium hydroxide, 4-ethylpyridine, 10% Pd on activated carbon, and acidic-type Al₂O₃ for chromatography were purchased from Fluka or Sigma-Aldrich. Polymers like chitosan, Alginic acid, polyacrylic acid, different PEG based monomers and polymers, different functional group polystyrene polymers are brought from Sigma-Aldrich and used as it is or modified according to requirement. The Ru precursor is hydrated ruthenium trichloride available from Johnson Matthey. The Os precursor is (NH₄)₂[OsCl₆] available from Alfa Aesar (99.99%). The Au precursor is Au(tetrahydrothiophene)Cl or Au(Me₂S)Cl, both of which the inventors synthesized from a pure gold metal dissolved in aqua regia followed by reduction using the tetrahydrothiophene or Me₂S ligand. Millipore® water was used as solvent for all studies.

Synthesis of gold phosphor: Anionic phosphorescent gold(I) compounds Na₈[Au(TPPTS)₃] and Cs₈[Au(TPPTS)₃] are synthesized exactly following literature procedure⁶⁶. The purity of all these complexes was ascertained via and ¹H and ³¹P{-¹H} NMR, IR, electronic absorption and emission, and time-resolved luminescence spectroscopic data compared to the literature.

Synthesis of the Pt(II) based or Ru(II) or Os(II) based phosphors: The phosphorescent Pt(II) complex commonly called “Pt—POP” was synthesized following the literature procedure¹¹⁸. The purity of the complex was ascertained via ¹H and ³¹P{¹H} NMR, IR, electronic absorption and emission, and time-resolved luminescence spectroscopic data compared to the literature. Different cationic and anionic Ru(II) based compounds like [Ru(bpy)₃](PF₆)₂ or K₄[Ru(dcbpy)₃], or K₄[Ru(tctrpy)₂] (where, bpy=2,2′-Bipyridine, dcbpy=4,4′-dicarboxy-2,2′-bipyridine; tctrpy=4,4′,4″-tricarboxy-2,2′:6′,2″-terpyridine) are synthesized and characterized exactly following literature procedure¹¹⁹. Os (II) analogues of the same are also synthesized and characterized using similar procedures already described in literature¹²⁰.

The structures of these Ru(II) and Os(II) based compounds are depicted schematically in FIGS. 4C-4E.

Synthesis of positively charged PNIPAM microgels: Positively charged PNIPAM microgels are synthesized using allylamine or other amine terminated monomers like N-(3-aminopropyl)methacrylamide hydrochloride as co-monomers following literature procedures¹³.

Synthesis of negatively charged PNIPAM microgels: Negatively charged PNIPAM microgels are synthesized using acrylic acid or other carboxylic acid terminated monomers as co-monomers following literature procedures¹³. Combination of both positively charged and negatively charged PNIPAM microgels are synthesized using equal ratios of co-monomers during syntheses.

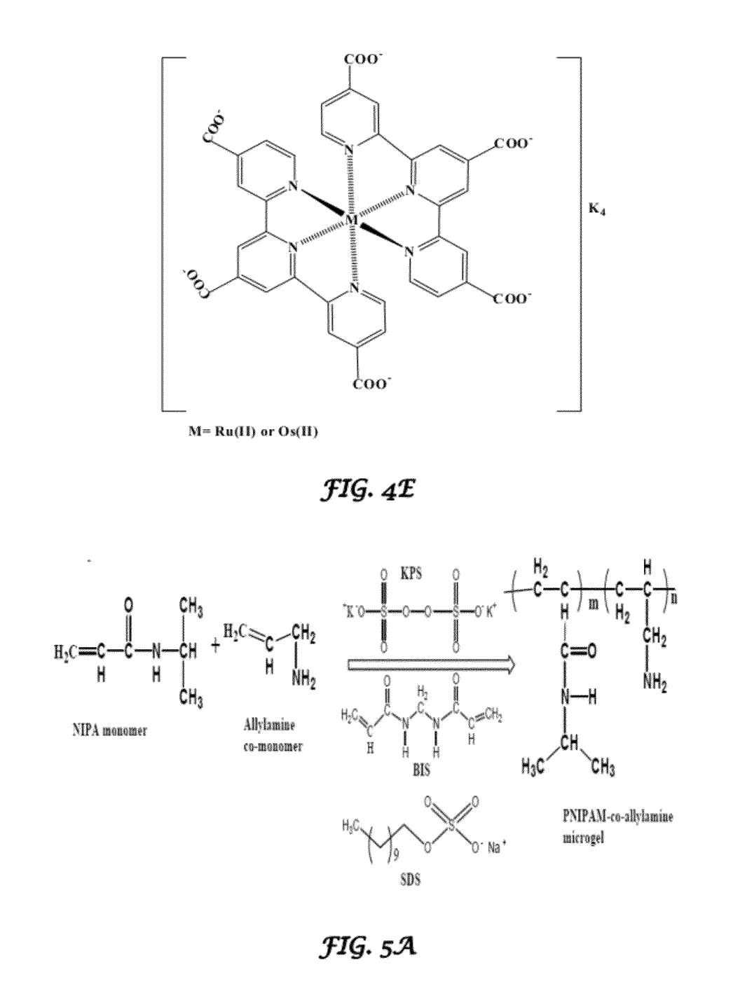

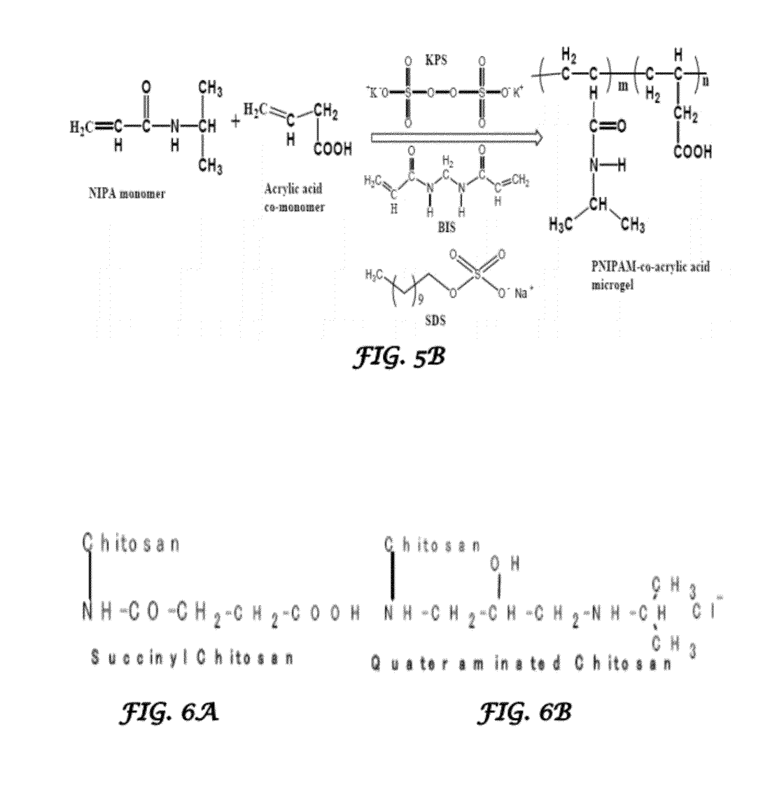

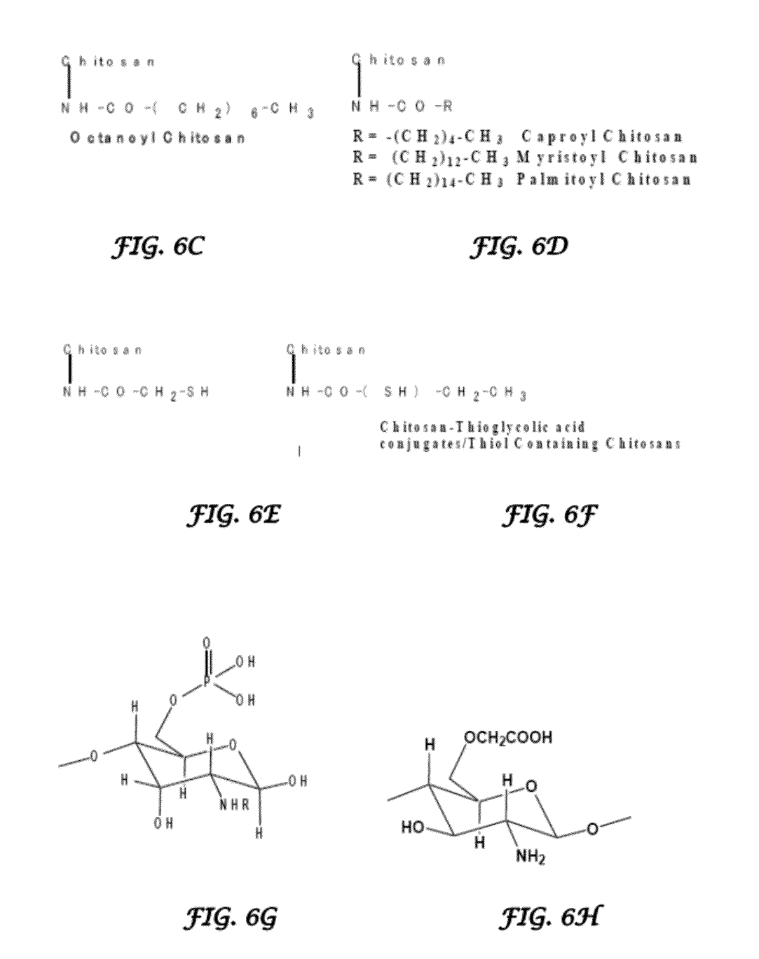

Chemically modified polymers: Different chemically modified polymers shown in FIGS. 6A-H and 7A-L are synthesized following literature procedures or brought directly from Sigma-Aldrich or Polyscience.

Synthesis of anionic gold(I) based phosphorescent PNIPAM-co-allylamine microgels: Loading of the phosphor was carried out by stirring 10 mL of a 1.5 wt. % aqueous solution of the relevant PNIPAM-allylamine microgel and 3 mL of a 0.001M aqueous solution of Na₈[Au(TPPTS)₃] for 48 hours under argon atmosphere, followed by centrifugation at 25000 rpm for 3 hours. After every one hour of centrifugation the supernatant was discarded and the sediment washed with Millipore® water. After three washes, the microgel obtained was incubated in DI water at around 22° C. for 2 days, allowing the formation of Au phosphor-loaded PNIPAM-co-allylamine or PNIPAM-co-acrylic acid microgel colloids or colloidal crystals.

Synthesis of anionic gold(I) based phosphorescent PNIPAM-co-allylamine hydrogel networks: Crystalline luminescent microgels in water obtained after incubation were crosslinked using di-glutaraldehyde as described elsewhere. Phosphorescent PNIPAM-co-allylamine crystalline microgel was added to di-glutaraldehyde at neutral pH followed by incubation at 4° C. for chemical crosslinking. After 48 hours, the fully crosslinked crystalline microgel network was removed and washed twice before storing in fresh Millipore water, resulting in formation of stable phosphorescent PNIPAM-co-allylamine engineered hydrogel crystal networks.

Formation of anionic gold(I) based lyophilized hybrid PNIPAM-co-allylamine microgel: The dispersion of crystalline arrays of phosphorescent microgels was freeze-dried. After 7 days, the freeze-dried microgel was characterized by luminescence spectroscopy at room temperature, and then re-dispersed in fresh Millipore water to check the loading stability of the gold phosphor. The re-dispersion was carried out by dipping the freeze-dried microgel into fresh Millipore water and then waiting for 24 hours to check the photoluminescence stability of the re-dispersed phosphorescent gel.

Synthesis of different anionic platinum based phosphorescent PNIPAM-co-allylamine microgels: As described in [0122], [0123], and [0124], different platinum (II) based phosphorescent microgels/hydrogels/lyophilized microgels are synthesized exactly in the same way but by using a Pt(II) based phosphor instead of Au(I) based phosphor.

Synthesis of different cationic gold(I) based phosphorescent PNIPAM-co-acrylic acid microgels: For stabilizing cationic gold(I) based phosphors (FIG. 2), the procedures described in [0122], [0123], and [0124] are employed but by using negatively charged PNIPAM microgels/hydrogels/lyophilized microgels instead of cationic PNIPAM gels.

Synthesis of phosphorescent polymer polyelectrolyte transition metal complex nano/microparticles: Polyelectrolyte complex phosphorescent polymeric particles are in general prepared by a simple dropping method. To a 5 ml 0.2 to 0.05 wt. % polymer solution (modified or unmodified Chitosan, PNIPAM, PAA, Alginic acid, Polystyrene, or Polyacrylic acid) is added drop by drop into the required concentration of polyionic transitional metal based phosphor with continuous stirring until an opalescent suspension was obtained. The resultant opalescent solution is first centrifuged at 15000 rpm followed by filtration using micron size millipore filters to separate any unreacted polymer. The samples are also subjected to sonication for better particle distribution. Finally the samples are dialyzed for 3 days to remove any unreacted polymers or unentrapped phosphors.

Phosphorescent positively charged polyelectrolyte complex micro/nanoparticles: As previously described in [0127], to the required wt. % of positively charged un-modified or modified polymers, different anionic transition/noble metal based phosphors such as those shown in FIGS. 1A and 1B are added in the required concentration to tune the size of polyelectrolyte complex particles anywhere between 20 nm to 1000 nm based on pH and concentration of anionic transition/noble metal phosphor complex.

Phosphorescent negatively charged polyelectrolyte complex micro/nanoparticles: As described in [0127], to the required wt. % of negatively charged un-modified or modified polymers polymers, different cationic transition/noble metal based phosphors like shown in FIG. 2 are added in required concentration to tune the size of polyelectrolyte complex particles anywhere between 20 nm to 1000 nm based on pH and concentration of cationic transition/noble metal phosphor complex.

Synthesis of polymer stabilized phosphorescent polyionic cyclic Au(I)/Ag(I)/Cu(I) trimer: First the cyclic metal based trimers (Au/Ag/Cu) are synthesized by dissolving modified azole (pyrazole/triazole/Imidazole) ligands shown in FIGS. 3A, 3B, 4A, and 4B in methanol or methanol/water mixture, followed by dissolving the metal precursor (Au(THT)Cl/Au(Me₂S)Cl/AgNO₃/Cu₂O) in another flask. After 2 hours of stirring both these solutions are mixed by addition of few drops of freshly distilled triethyl amine. The mixture is left stirring unexposed to light or air for 3 more hours. The formation of cyclic trimer can be checked from emissive color of the solution. The final solution is filtered and recrystallized. After recrystallization the cyclic trimer can be dissolved in any polyionic biopolymer/thermosensitive polymer solutions by simple stirring for over 6 hours. After 6 hours the solution is filtered and centrifuged to collect the emissive sediment.

In situ stabilization/synthesis of phosphorescent polyionic Au(I)/Ag(I)/Cu(I) cyclic trimer complexes: Modified Pyrazole/Triazole/Azole ligands after dissolving in water/methanol mixture, is added to any polyionic biopolymer/thermosensitive polymer maintaining the pH of the solution either 3.0 or 6.0 by addition of required amount of acetic acid or ammonium hydroxide. After stirring the ligand with polymer solution for 3-4 hours required amount metal precursor is added followed by stirring under ambient conditions for 24 hours. The unreacted polymer and metal precursor are removed after repeated centrifugation. In situ formation of cyclic trimers with in polymer systems can be indicated from their strong emissive colors. Later the solution is centrifuged to collect emissive sediment, used as it is for heavy metal ion sensing.

Synthesis of positively charged polymer stabilized phosphorescent anionic cyclic Au(I)/Ag(I)/Cu(I) trimer systems: As described previously, for stabilizing anionic Au(I), Ag(I), Cu(I) cyclic trimer systems, any of the positively charged unmodified-modified polymers mentioned above are used.

Synthesis of negatively charged polymer stabilized cationic cyclic Au(I)/Ag(I)/Cu(I) trimer systems: As described previously, for stabilizing cationic Au(I)/Ag(I)/Cu(I) trimer systems any of the negatively charged unmodified-modified polymers mentioned above are used.

Synthesis of cationic [Ru(bpy)₃](PF₆)₂ based phosphorescent PNIPAM microgels: Cationic [Ru(bpy)₃](PF₆)₂ is stabilized in different negatively charged PNIPAM microgels in the same way as described in previous sections above.

Synthesis of anionic K₄[Ru(dcbpy)₃] based phosphorescent PNIPAM microgels: Anionic K₄[Ru(dcbpy)₃] (dcbpy=4,4′-dicarboxy-2,2′-bipyridine) is stabilized at high pH in various positively charged PNIPAM microgels exactly in the same way as described previously.

Synthesis of anionic K₄-[Ru(tctrpy)₂] based phosphorescent PNIPAM microgels: Anionic K₄[Ru(tctrpy)₂] (tctrpy=4,4′,4″-tricarboxy-2,2′:6′,2″-terpyridine) is stabilized at high pH in various positively charged PNIPAM microgels exactly in the same way as described in the previous sections.

Phosphorescent positively charged polyelectrolyte complex micro/nanoparticles using Anionic K₄-[Ru(dcbpy)₃] or K₄-[Ru(tctrpy)₂] as physical crosslinker: As described previously to required wt. % of positively charged un-modified or modified polymers like chitosan/NIPA, required concentration of K₄[Ru(dcbpy)₃] or K₄[Ru(tctrpy)₂] is added to tune the size of polyelectrolyte complex particles anywhere between 20 nm to 1000 nm based on pH and concentration of anionic phosphor complex.

Phosphorescent negatively charged polyelectrolyte complex micro/nanoparticles using cationic [Ru(bpy)₃](PF₆)₂ as crosslinker: As described in the previous sections to required wt. % of negatively charged un-modified or modified polymers like polyacrylic acid/alginic acid, required concentration of [Ru(bpy)₃](PF₆)₂ is added to tune the size of polyelectrolyte complex particles anywhere between 20 nm to 1000 nm based on pH and concentration of cationic phosphor complex.

Synthesis of cationic [Os(bpy)₃](PF₆)₂ based phosphorescent PNIPAM microgels: Cationic [Os(bpy)₃](PF₆)₂ is stabilized in different negatively charged PNIPAM microgels exactly in the same way as described in previously.

Synthesis of anionic K₄[Os(dcbpy)₃] based phosphorescent PNIPAM microgels: Anionic K₄[Os(dcbpy)₃] (dcbpy=4,4′-dicarboxy-2,2′-bipyridine.) is stabilized in different positively charged PNIPAM microgels exactly in the same way as previously described.

Synthesis of anionic K₄[Os(tctrpy)2] based phosphorescent PNIPAM microgels: Anionic K₄[Os(tctrpy)₂] (tctrpy=4,4′,4″-tricarboxy-2,2′:6′,2″-terpyridine.) is stabilized in different positively charged PNIPAM microgels exactly in the same way as described in the previous section.

Phosphorescent positively charged polyelectrolyte complex micro/nanoparticles using Anionic K₄[Os(dcbpy)₃] or K₄[Os(tctrpy)₂] as crosslinker: As described previously to required wt. % of positively charged un-modified or modified polymers like chitosan/NIPA, required concentration of K₄[Os(dcbpy)₃] or K₄[Os(tctrpy)₂] is added to tune the size of polyelectrolyte complex particles anywhere between 20 nm to 1000 nm based on pH and concentration of anionic phosphor complex.

Phosphorescent negatively charged polyelectrolyte complex micro/nanoparticles using cationic [Os(bpy)₃](PF₆)₂ as crosslinker: As described in the previous sections to required wt. % of negatively charged un-modified or modified polymers like polyacrylic acid/alginic acid, required concentration of [Os(bpy)₃](PF₆)₂ is added to tune the size of polyelectrolyte complex particles anywhere between 20 nm to 1000 nm based on pH and concentration of cationic phosphor complex.

Photoluminescence characterization: All photoluminescence studies were carried with the PTI spectrofluorometer described below. For titrations, 2.5-mL aliquots were placed in the quartz cuvette and 0.1 mL of gel was added at every titration point with magnetic stirring. Titration data were plotted as I/Iₒ vs. volume of gel added (where I=intensity of sample after addition of gel and 1ₒ=intensity of pure aqueous gold phosphor solution). Lifetime data were analyzed using a xenon flash lamp or N₂ laser systems for both the sample and a scatterer solution used to generate the instrument response function (IRF) used for deconvolution.

Dynamic light scattering characterization: The hydrodynamic radius of each sample was measured by dynamic light scattering (DLS). The temperature of the samples was controlled by a circulation water bath (Brinkmann Lauda Super RM-6) to within ±0.02° C. The samples for all the dynamic light scattering analysis were prepared by homogenization followed by dilution with Millipore water. Each sample was measured 3 times and the mean radius was reported. Zeta potential was measured on a zetasizer nano ZS (Malvern Instruments) loading samples into maintenance-free cells.

Spectroscopic equipment: Steady-state luminescence spectra were acquired with a PTI QuantaMaster Model QM-4 scanning spectrofluorometer equipped with a 75-watt xenon lamp. The UVN is absorption spectra were measured at 1-nm resolution on either a diode array spectrometer (Hewlett-Packard, model 8543) or a Perkin-Elmer Lambda-900 double-beam UVN is/NIR absorption spectrophotometer.

UV-Visible spectroscopy measurements: The turbidity (α) of the dispersions was measured vs. wavelength using a diode array UV-Visible spectrometer (Agilent 8453) by calculating the ratio of the transmitted (Iₜ) to incident (I_{O}) light intensity, α=−(1/d)In(Iₜ/I₀), where d is the thickness (1 cm) of the sampling cell.

Spectroscopic characterization of the gold phosphor: Na₈[Au(TPPTS)₃] comprises a highly water soluble three-coordinate Au(I) anionic complex that exhibits phosphorescence at room temperature both in aqueous solution and the solid state. According to the literature, Na₈[Au(TPPTS)₃] exhibits a broad emission band maximum at 494 nm in the solid state at room temperature, whereas at 77° K the emission intensity increases and undergoes a blue shift in peak maximum to 486 nm. In aqueous solution, the [Au(TPPTS)₃]⁸⁻ emission is red-shifted with unsymmetrical broad emission band centering approximately at 515 nm. The absorption and photoluminescence spectra obtained for the gold phosphor samples synthesized herein were such that an aqueous solution of 0.001M [Au(TPPTS)₃]⁸⁻ exhibits a strong emission maximum at 525 nm and excitation maximum at 292 nm. The broad green emission band in solution is blue-shifted to turquoise-blue in the solid state with unsymmetrical broad emission at 500 nm with excitation maximum at 356 nm. The photophysical properties observed for our samples synthesized are in overall good agreement with those reported by Assefa et al⁶⁶.

Luminescence spectra of different hybrid hydrogel forms: The gold phosphor has been successfully loaded into three forms of PNIPAM-co-allylamine hydrogel systems: microgels in water, chemically-crosslinked bulk hydrogel, and lyophilized freeze-dried xerogel. FIG. 11B shows that these different forms of hydrogel systems after incorporating the gold phosphor exhibit similar photoluminescence spectra to those for the gold phosphor itself in aqueous solution or the solid state. This result suggests that the phosphor molecule retains its optical properties in its native form even in presence of different hydrogel environments, which would be very attractive for different biological applications as most of the fluorescent dyes are expected to be sensitive to changes in microenvironment. Gold phosphor-loaded PNIPAM-co-allylamine microgel dispersion (FIG. 11B (i)) and the bulk hydrogel (FIG. 11B (ii)) exhibit broad unsymmetrical green emission with λₘₐₓ near 525 nm, mirroring the emission of an aqueous solution of the phosphor. An increase in emission intensity accompanied with a blue shift to 490 nm is observed in the lyophilized freeze-dried xerogel (FIG. 11B (iii)) at room temperature. The characteristic broad unsymmetrical emission bands from the gold phosphor are retained with minor shifts. Previous studies based on quantum mechanical computations illustrated the feasibility of systematic tuning of emission by controlling steric bulk in very similar three-coordinate Au(I) complexes, suggesting a Jahn-Teller distortion from a trigonal-planar ground state toward a T-shaped phosphorescent excited state. PNIPAM microgels and bulk hydrogels in their swollen form in aqueous solution allows for a relatively unencumbered distortion toward the T-shape geometry of the phosphorescent state, whereas the freeze-dried xerogel form entails a significant constraint to such a large molecular distortion. The underlying “luminescence rigidochromism” phenomenon in such three-coordinate d¹⁰ systems is addressed at a fundamental level elsewhere for water-insoluble complexes;⁶⁹ the phenomenon is well-known for other classes of phosphorescent molecules, including their use to monitor sol-gel-xerogel and other setting transformations in inorganic silicates⁷⁰⁻⁷⁷.

Influence of the gold phosphor on inherent hydrogel properties: PNIPAM-co-allylamine microgels in water with polymer concentrations within 1.5-5.0 wt. % can self-assemble into colloidal arrays with bright iridescent patterns at room temperature discussed elsewhere in detail. Even after loading the gold phosphor, hybrid microgels at a concentration of about 2.0 wt. % self-assemble into a temperature sensitive ordered crystalline arrangement. Because of the Bragg diffraction from the crystalline phase at 21° C., the UV-visible spectrum exhibits a sharp attenuation peak (FIG. 13), measured with UV-visible spectroscopy. At an elevated temperature (24° C.), both the peak height and peak wavelength decrease with time, indicating melting of crystalline structures. The shift in the sharp Bragg diffraction peak and its disappearance upon temperature increase in the hybrid phosphorescent microgels here is remarkably similar to the behavior with the unloaded PNIPAM microgel systems. This result signifies that the presence of gold phosphor does not result in sacrificing either formation or stimuli responsiveness of PNIPAM microgel crystals.

The magnitude of electrostatic attraction or repulsion is considered a key factor in controlling the dispersion mechanism of colloids in general. Loading the gold phosphor into PNIPAM-co-allylamine microgel dispersions due to electrostatic interactions in the aqueous medium is explained based on changes in dynamic light scattering data, zeta potential values, pH and functional group-dependent luminescence data obtained from various microgels studied at room temperature. Previous work by Frisken and co-workers illustrated a sharp decrease in particle size by introducing charged or ionic components into similar PNIPAM microgel systems. FIG. 12A shows the hydrodynamic radius changes in PNIPAM-co-allylamine microgels after loading the gold phosphor, giving rise to a 23% decrease in Rh values, from 118 nm to 92 nm, under identical concentration and experimental conditions. Strong electrostatic interactions between the microgel particles and the transition metal phosphorescent complex also result in improved particle distribution, as clearly manifested by the reduction in the width of the DLS peak of the microgel dispersion after loading the phosphor (FIGS. 12A and 12B).

The changes in hydrodynamic radius emphasize charge component interactions between the transition metal based phosphor and PNIPAM-co-allylamine microgel particles as observed elsewhere in similar microgel particles. Examination of zeta potential values also confirms the same conclusion. Thus, the +25.1 mV zeta potential value for the microgel at pH 5.0 before loading, which is attributed to positively-charged protonated allylamine groups of microgel dispersion decreases to +5.3 mV immediately after adding the gold phosphor. This decrease in zeta potential values in hybrid microgels clearly indicates adsorption of negatively-charged phosphorescent complexes onto positively-charged microgel spheres. Based on these results, the inventors assume strong polyelectrolyte interactions between PNIPAM-co-allylamine microgel and the gold phosphor. FIG. 8A illustrates that the anionic Au(I) complex can effectively screen the interaction between positive charges on the microgel and lead to size reduction of the microgel. It is noted that other factors may also contribute the reduction of the particle size. For example, for charged colloids the diffusion coefficient is larger at lower ionic strengths, which corresponds to smaller size.

The crystallinity of PNIPAM microgel crystals can be lost by shaking the dispersion at room temperature or heating the colloidal crystals just above room temperature. To stabilize the colloidal structure, the crystalline array of gold phosphor-loaded microgel particles was covalently-crosslinked with di-glutaraldehyde following a published procedure. The resulting hybrid bulk hydrogel network (FIG. 8B) not only has a stable colloidal crystalline structure but also retains the broad green emission peak unchanged at 525 nm (FIG. 11B (ii)). This suggests the presence of a strong electrostatic attraction between the PNIPAM-co-allylamine microgel and the gold phosphor. To the best of the present inventor's knowledge, this is the first example of a luminescent crystalline network that has been successfully obtained in aqueous medium.

Photoluminescence spectra recorded at room temperature for the lyophilized sample (FIG. 11B (iii)) show a broad turquoise emission with maximum at 481 nm, similar to that for the gold phosphor solid. These spectra, however, are significantly different from those of the hybrid microgel aqueous samples with a significant blue shift. The lyophilized hybrid microgel can be re-dispersed into fresh Millipore water, re-generating the green emission characteristic of the aqueous microgel (FIG. 11B (iii)), bottom-right inset. These findings demonstrate stable loading of the gold phosphor into PNIPAM-co-allylamine microgels both in the solid and solution phase.

Functional group and pH dependence of phosphor loading: Guided by the steady state photoluminescence data, loading of the gold phosphor into PNIPAM microgels is determined to depend on both the identity of the co-monomer and the pH of the microgel solution. Loading studies were conducted for PNIPAM microgels with allylamine and acrylic acid co-monomer functionalities at acidic and basic pH values. FIGS. 14A-14D show steady-state photoluminescence data for the gold phosphor loaded under these variations. The pH of the PNIPAM-co-allylamine microgel dispersion was adjusted from acidic values within 5.5-4.0 to basic values near 9.0 by addition of suitable volumes of 0.1M acetic acid and 0.1M ammonium hydroxide solutions, respectively, to the microgel dispersion. The efficiency of the gold phosphor loading can be readily determined by contrasting the photoluminescence intensity of supernatants vs. sediments in each of the four samples studied. The most efficient loading is observed in PNIPAM-co-allylamine microgels at pH 4.0, whereas very little or no loading is observed in PNIAPM-co-acrylic acid microgels at pH 9.0.

PNIPAM-co-allylamine microgels at pH 4.0 exhibit efficient loading of the gold phosphor. But for the same system at pH 9.0, the PL data indicate incomplete or inefficient loading. In the case of acrylic acid-based microgels at pH 9.0, on the other hand, all the gold phosphor was retained in the supernatant while the sediment did not exhibit any emission, indicating 0% loading. At pH 4.0, the same acrylic acid microgel shows <20% emission intensity from the sediment of the microgel while most of the gold phosphor was still in the supernatant without being loaded. So its concluded that from DLS, zeta potential and steady state data attachment/loading of transition/Noble metal based phosphor on to microgel is, therefore attributed to electrostatic interactions between negatively charged anionic groups of phosphor and positively charged microgel or vice-versa. A previous report by Gong et al. showed the entrapment of photoluminescent quantum dot (QD) nanocrystals into PNIPAM microspheres due to centrifugation force and weak hydrogen bonding between thioglycerol-capped CdTe nanocrystals and PNIPAM microspheres⁷⁸; the stable fluorescent microspheres were observed after centrifugation only in the sediment compared to the supernatant of the sample. Uniform size of the nanocrystals and aggregation were controlling factors for loading such QD nanocrystals into PNIPAM microspheres. However, the present results suggest that such uniformity and aggregation problems can be avoided for loading any polyelectrolytic molecular phosphors like Na₈[Au(TPPTS)₃] into oppositely charged PNIPAM/other above mentioned soft based gels/microspheres.

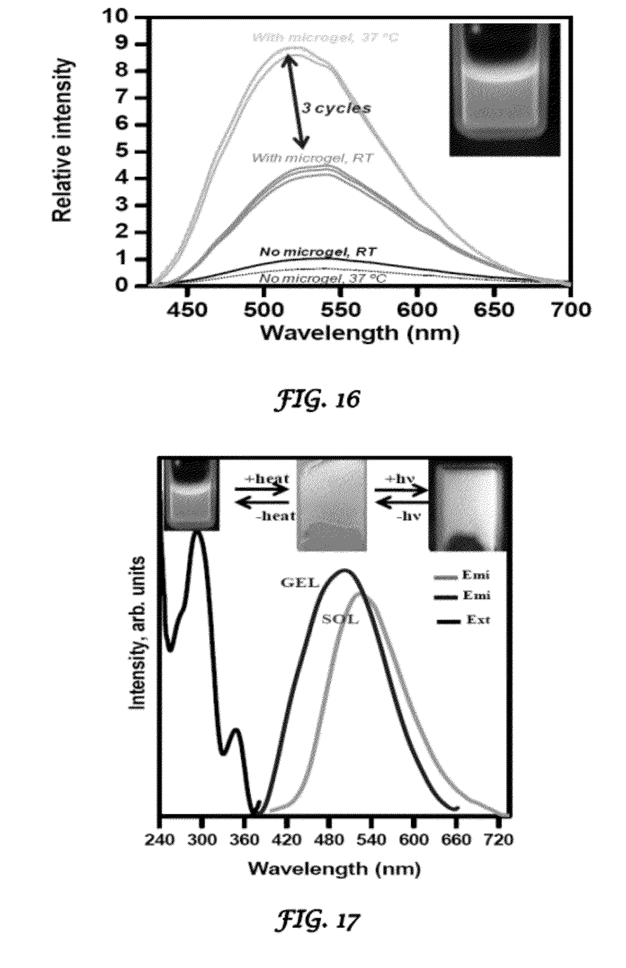

Photoluminescence enhancement: One can attain significant photoluminescence (PL) enhancement in multiple fashions upon incorporation of Noble/Transition metal based phosphors into different stimuli responsive polymers/microgels. For example a dramatic manifestation is noticed upon titrating the gold phosphor (Na₈[Au(TPPTS)₃]) with PNIPAM-co-allylamine microgels. The extent of PL enhancement can be varied by controlling the gold phosphor concentration, temperature above or below the volume phase transition temperature of the microgel, pH, etc. Titration experiments at various gold phosphor concentrations while keeping the concentration of the microgel constant are shown in FIGS. 14A-14D while the effect of the microgel concentration is shown in FIGS. 15A-15D. The highest ratio of PL enhancement is attained at the lowest concentration of gold phosphor tested. In this set of experiments, we attain a 4-fold, 9-fold, 14-fold, 17-fold, and 50-fold PL enhancement upon decreasing the gold phosphor concentration along the direction 5×10⁻⁵ M, 1×10⁻⁵ M, 5×10⁻⁶ M, 3×10⁻⁶ M, and 1×10⁻⁶ M, respectively. The inventors state that, at a fixed microgel concentration, only a certain number of phosphorescent molecules will be loaded or are present in the vicinity of the microgel surface so as to contribute to the PL enhancement. Titration experiments were also performed at basic (pH 9.0) conditions. The microgel concentration affects the PL enhancement in all cases. For example, FIGS. 15A-15D show gradual PL enhancements up to 70-fold on addition of 0.2 mL of microgel while further microgel aliquots lead to slow decrease or a plateau; the inner-filter effect becomes relevant at higher gel concentrations⁷⁹⁻⁸². A freshly synthesized sample of the Au phosphor was titrated with smaller quantities of microgel to diminish the inner-filter effect while increasing the PL enhancement up to 157-fold (FIGS. 15C and 15D). These results signify that, at a fixed concentration of phosphor, highest PL enhancement is attained at a critical concentration of microgel, which is very similar to the micellar effect on fluorescence enhancement as noticed by Takeuchi. The collection of experiments we performed suggests delicate interplay between the critical microgel concentration, concentration of the gold phosphor, pH, temperature, ionic strength, sample freshness, molecular weight and refractive index of polymer gel, etc. An interesting situation is shown in FIG. 16 in which a blue shift in emission maximum, from 532 nm to 515 nm, concomitant with emission enhancement, is seen upon heating microgels from ambient temperature to around 37° C. The luminescence data in FIG. 16 suggests that the physical state of heated hybrid microgel sample is intermediate between solution form and the lyophilized dried form. This result is consistent with the luminescence rigidochromism phenomenon discussed above for three-coordinate Au (I) complexes. Thus, the Jahn-Teller T-shape distortion in the phosphorescent excited state from the trigonal ground state geometry becomes more greatly hampered as one proceeds from the fluid solution, to the more viscous heated gel, and then to the lyophilized dried gel, leading to greater blue shift in that direction. As shown in the temperature-dependent titrations in FIG. 16, heating the gold phosphor microgel system to 37° C. results in not only a 620 cm⁻¹ blue shift in emission maximum but also approximately 100% PL enhancement (double the intensity based on peak height). This thermally-induced PL enhancement is fully reversible, as shown by three heating/cooling cycles in FIG. 16. The PL enhancement on heating is at least partially ascribed to the change of the microenvironment from hydrophilic to hydrophobic as PNIPAM is heated above its lower critical solution temperature (LCST, which is about 34° C.). The PL enhancement is likely further assisted by an increase of the refractive index of the microgel when the temperatures are higher than the LCST, as observed previously in III-V semiconductor QD- or ZnO-embedded PNIPAM microgels⁸³⁻⁸⁴. The control study for an aqueous solution of the gold phosphor in absence of the PNIPAM microgel leads to quenching upon heating from ambient temperature to 37° C., as expected due to increased non-radiative decay by multiphonon de-excitation to the ground state. This result is promising for possible utilization of the phosphorescent hybrid systems here for live bioimaging applications, for which a strong PL signal at the physiological temperature of 37° C. is critically important.

The above findings are contrasted with other relevant studies in the literature. Photoluminescence was reported earlier in lanthanide-doped hydrogel systems due to energy transfer from PNIPAM-co-styrene to Tb(III) or MMA:HEMA to Eu(III) phosphorescent centers, whereas the systems herein do not contain a chromophore in the hydrogel host as the transition metal phosphorescent center contains chromophoric ligands already. The Eu(III)-doped MMA:HEMA hydrogel films exhibited pH-dependent photoluminescence due to populating the lanthanide excited state by sensitization, whereas here temperature, concentration of phosphor, polymer, microgel all play major roles in the phosphorescence sensitization in addition to pH. Notable literature precedents included the work of Li et al. in which a 4% PL enhancement was reported from different size quantum dots embedded in thermosensitive PNIPAM gels at room temperature. These authors attained nearly 10-fold quenching upon heating the PNIPAM/QD hybrid gels to near body temperature, unlike the dramatic enhancement we obtain in this work (FIG. 16). Zhou and co-workers observed a 1.72-fold PL enhancement for terbium citrate upon binding to silver nanoparticles in solution, attributed to electric field enhancement around terbium from the electron plasmon resonance of silver nanoparticles. Takeuchi reported more significant PL enhancements of 8-20× for dansyl amino acid probes in presence of different surfactants due to micellar effects. The 1-2 order-of-magnitude PL enhancement seen herein in aqueous hydrogel media is rivaled only in purely organic media, which are usually less susceptible to quenching than aqueous media; e.g., a 40-fold PL enhancement for pyrene was reported upon embedding the fluorophore in hydrophobic polystyrene (toluene solution).

Phosphorescent transition metals have been incorporated in gel soft materials besides hydrogels, e.g., as described in the work of Dunn and Zink for Re(I) complexes in orthosilicate sol-gel systems, Aida and co-workers for Ag⁺ adduct^{s} of trinuclear Au(I) complexes with 4-(3,5-dioctadecyloxybenzyl)-3,5-dimethylpyrazole organogels,⁸⁵ and Yam and co-workers for Pt(II) alkynyl complexes in 2,6-bis(N-dodecylbenzimidazol-2′-yl)pyridine⁸⁶. The present inventors are unaware of any precedents of hydrogel/microgel/polymer environments entrapping molecular or semiconductor species to exhibit photoluminescence in such a broad range of gel forms and/or responsiveness to the range of study variations reported herein.

As for the origin of PL sensitization, there are several mechanisms that have been invoked in prior literature precedents. Various groups reported that when a fluorophore is positioned within a restricted space provided by micelles or metallic nanoparticles, the electronic absorption and fluorescence spectra often change due to the effect of the microenvironment on fluorophores. Increase in structural rigidity accompanied by decrease in accessibility to the surrounding aqueous medium in presence of micelles decreases the non-radiative deactivation, resulting in fluorescence enhancement in fluorophores positioned within the microenvironment of micelles or vesicles. In case of fluorophores localized close to metallic nanoparticles, fluorescence enhancement is explained as being due to intensified electromagnetic field from electronic plasmons, resulting in increased excitation rate or radiative rates. Finally, an increase in the refractive index of the microgel was invoked to explain PL enhancement in PNIPAM microgels embedded with III-V semiconductor quantum dots or ZnO nanoparticles. These different explanations are all relevant to the PL enhancement observed herein. PNIPAM hydrogels indeed offer a micelle environment because they contain both hydrophilic and hydrophobic parts, thus limiting the quenching by water molecules compared to analogous aqueous solutions of noble/transition metal based phosphors that do not contain hydrogel/any polymer matrix systems. Both the electrostatic interactions (between the anionic/cationic phosphorescent complex and oppositely charged soft gel or polymer system) and van der Waals or other hydrophobic/hydrophilic interactions (between hydrocarbon and other non-polar parts of the phosphor and PNIPAM/polymer moieties) act to reduce water quenching and thus the rate of non-radiative decay. In addition to the drastic PL enhancements seen in FIGS. 14A-14D, 15A-15D, and 16 the inventors observed an increase of the phosphorescence lifetimes (from 1.4 μs to 2.6 μs in a typical example of (Na₈-[Au(TPPTS)₃]) for solutions that exhibit an order-of-magnitude PL intensity enhancement on addition of PNIPAM hydrogels to the aqueous solutions. The magnitude of the lifetime increase is less than the corresponding intensity increase, suggesting that suppressed non-radiative deactivation via multiphonon relaxation to the ground state is not solely responsible for the PL enhancement observed. Therefore, gel/polymer scattering must play at least some role in the PL sensitization, consistent with multiple literature precedents for other classes of emitters⁸⁷⁻⁸⁸.

The present invention discloses stimuli-sensitive phosphorescent hydrogel microspheres synthesized by incorporating a transition/specially noble metal based coordination compounds, like Na₈-[Au(TPPTS)₃], phosphor into PNIPAM or other soft based gels or above mentioned polymers. The resulting hybrid material exhibited sensitized Au-centered emission compared to that of the gold complex in water both above and below the volume phase transition temperature of the microgel. The resulting phosphorescent microspheres showed decreased size and PL enhancement with particularly high phosphorescence sensitization at physiological pH and temperature. The results show strong dependency of the phosphor loading on pH and nature of functional group to maximize the interactions between the PNIPAM microgel host and the gold phosphor guest. The results presented herein encourage the development of new classes of water-soluble transition metal complexes which can serve multiple functions to act as phosphorescent physical crosslinkers of various biopolymers, biological labeling reagents for imaging, and/or optical sensors for various biological and environmental applications.

In addition to the studies on loading water soluble transition/noble metal based gold(I) phosphors^{65,67,103} such as Na₈-[Au(TPPTS)₃], into different soft gels or polymer based dispersions as discussed hereinabove, the present inventors report thermoreversible gelation from PNIPAM-co-allylamine microgels with retained photoluminescent properties on doping with AuP. This system is a liquid at room temperature but becomes a gel/aggregate upon heating close to body temperature (˜37° C.). The inventors further show that a polyanionic gold based phosphor can physically crosslink positively charged polymer chains into microgels. As a result, this invention reveals that polyanionic transition/noble metal based phosphors have dual roles: one acts as a physical crosslinker that results in gelation above the LCST or links positively polymer chains into microgels, and the other performs as light emitting centers.

It is well known that luminescent transition metal complexes can serve as biological labels and probes because of their intensive long-lived emissions in the visible region¹⁰⁴. Different metal based phosphors were studied¹⁰⁴⁻¹⁰⁶ to surpass disadvantages like short nanosecond lifetimes, self-quenching effects, high photo-bleaching, and weak fluorescence intensities at physiological pH of conventional fluorescent labels through various emission enhancement mechanisms^{59,60,62}. The present inventors are herein disclosing doping and emission enhancement from transition/noble metal phosphor in microgel dispersions by more than an order of magnitude compared to the situation in a plain aqueous medium at different temperatures, which would encourage using these phosphors for labeling studies of soft material in particular.

On the other hand, polymer solutions with a thermoreversible gelation are highly sought after because it may be used as an injectable scaffold for delivering medicine or encapsulating cells⁴. A typical system is gelatin solution that undergoes normal sol-gel transition without chemical cross-linking^{5,6}. Other polymeric solutions have an inverse (that is, gelation upon heating) thermoreversible gelation including triblock copolymers (Pluronic or Poloxamer) or degradable triblock copolymers^{7,8}. Recently, an inverse thermoreversible gelation has been reported by the present inventors from PNIPAM-PAAc IPN microgels⁹ and Gan and co-workers have reported in-situ gelation using (P(NIPAM-HEMA)) based microspheres as building blocks in presence of CaCl₂ and used them as injectable scaffolds in cell encapsulation studies¹⁰¹. In all these cases, though physical gelation/aggregation/flocculation behavior in PNIPAM and other block polymers is attributed to physical interactions between oppositely charged polymer systems or due to presence of simple electrolytes like NaCl or NaNO₃ or CaCl₂ at high concentrations (˜0.08M), but never using a phosphorescent molecular system as luminescent gelating agent as reported here.

Different weight percentage (wt. %) PNIPAM-co-allylamine microgels prepared using precipitation polymerization method⁹ are mixed with different concentrations of AuP in a culture tube at room temperature and then placed in a water bath at regulated temperature 36+/−1° C. The physical inverse thermoreversible gelation is observed within a few minutes by inverting the test tube upside down. (FIG. 10) Visual observations also revealed that solutions turned from translucent to white precipitate during inverse gelation (FIG. 10).

Compositions of both microgel and gold phosphor required for exhibiting inverse gelation are summarized in Table 1. It is noted that at microgel concentrations lower than 1.5 wt. % gelation/aggregation is not observed even with highest concentrations of gold phosphor tested (0.005 molar) but whereas at 2 wt. % of the microgel inverse gelation can be tuned, and with beyond 3 wt. % of the gel at highest concentrations of AuP tested strong physical aggregation is noticed which is not reversible for a few hours. So the process of inverse thermo reversible gelation can be tuned from a point of no gelation to strong inverse thermo reversible gelation by varying concentration of both AuP and microgels.

TABLE 1
Concentration dependent gelation observed by inverting a test tube.

| | 1.5% | 2.0% | >3 wt % |
|---|---|---|---|
| 5 × 10⁻³M | Weak | YES | Strong |
| 10⁻³M | NO | YES | YES |
| 10⁻⁴M | NO | NO | NO |

The mechanism of inverse thermoreversible gelation is investigated using a dynamic light scattering (DLS) method that provides valuable insight into the onset and growth of cluster sizes. Upon heating the sample, hydrodynamic radius increases (Rₕ) (Table 2) as nanometer size microgel particles in the sol state start to grow into micron size aggregates indicating onset of gelation/aggregation (FIG. 10). From Table 2, weak gelation is indicated with 8.2 times increase in hydrodynamic radius at 0.001 molar AuP concentration compared to 22 times increase in radius in case of strong gelation at 0.01 molar AuP concentration. The controls show regular decrease in particle size with temperature for samples with 0.0001 or lower molar concentrations of AuP that exhibit no gelation similar to undoped microgel (FIG. 35).

TABLE 2
Determining strong, weak and no gelation in PNIPAM-co-allylamine microgels
at various molar concentrations of AuP by dynamic light scattering data.

| | Strong Gelation (Rₕ) | | Weak Gelation | | | |
|---|---|---|---|---|---|---|
| Temperature | nm | | (Rₕ) nm | | No Gelation | |
| (° C.) | undoped | hybrid | undoped | hybrid | undoped | hybrid |
| 21 | 118 | 92 | 117 | 92 | 114 | 90 |
| 24 | 109 | 90 | 115 | 89 | 110 | 87 |
| 27 | 107 | 84 | 108 | 82 | 106 | 82 |
| 30 | 99 | 58 | 99 | 65 | 100 | 73 |
| 34 | 80 | 86 | 72 | 75 | 96 | 61 |
| 36 | 57 | 1870 | 54 | 610 | 69 | 43 |

The inverse thermoreversible gelation in oppositely charged microgels/polymers and phosphor systems are not understood completely but predicted due to both van der Waal interaction between neighboring microgels above the LCST and electrostatic interactions between oppositely charged systems, similar to mechanism explained by Benee et al. and Islam et al. individually to explain irreversible and reversible flocculates formed from PNIPAM microgels in presence of different electrolyte solutions¹⁰⁷. In another similar work Saunders et al., have demonstrated effect of NaNO₃ on PNIPAM graft copolymer aggregate formation¹⁰⁷⁻¹⁰⁹. Thus the attractive interactions between dispersed microgels and AuP play an important role for the gelation. Therefore increased concentration of polyanionic gold phosphor inside PNIPAM-co-allylamine matrix results in strong aggregation, resulting in larger macroscopic aggregates as confirmed from dynamic light scattering data in Table 2. It is clearly noticed that the gelation temperature is directly related to the LCST of the PNIPAM in either weak gelation or strong gelation samples. This is because the chemical structure of PNIPAM microgel particles remains the same in presence of phosphor.

The PNIPAM-co-allylamine/AuP system not only has the inverse thermoreversible gelation behavior but also exhibits photoluminescent properties. FIG. 17 shows excitation dependent emission tuning in hybrid microgels which is not observed from Na₈-[Au(TPPTS)₃] itself in plain aqueous solution, the hybrid microgel dispersion exhibits retained broad unsymmetrical emission at ˜527 nm in sol state and on gelation turquoise emission at ˜502 nm with 20 nm blue shift is observed. Systemic emission tuning in three coordinate gold (I) complexes is explained due to excited Jahn-Teller distortion. The reversible blue shift in emission from 527 nm to 502 nm (green to turquoise) between sol-gel state was anticipated due to T-shaped distortion of gold phosphor in gel state as the matrix of the microgel becomes rigid on gelation compared to sol state coupled with aggregation of phosphor molecules in gel form attaining an intermediate state (gel) between bulk solution and solid phase that decreases the distortion in AuP resulting in emission blue shift. Though a good number of photoluminescent sol-gel probes^{74,76} are reported but never before in aqueous soft matter systems like PNIPAM hydrogels from gold based luminophores, so the inventors predict luminescence spectroscopy that is routinely employed for detecting phase transformations in polymers and gels in presence of different chromophores⁹⁷, can further be utilized to detect changes in rigidity and structural changes of polymer/gel matrixes in presence of similar gold based/other transition metal based luminophores that exhibit rigidochromism are capable of acting as gelating agents especially in soft materials.

The inventors also describe the application of these transition/noble metal based phosphors as physical crosslinkers to link positively/negatively charged polymer chains into microgel dispersions. Two positively charged polymer chains have been demonstrated here: one is thermo-responsive PNIPAM-RCONH₂, and the other is pH sensitive chitosan (CS), a natural biologically benign polymer. FIGS. 9A and 9B show the general ionic interactions between positively charged amine groups of chitosan/PNIPAM-RCONH₂ polymers and anionic sulfonate groups of Na₈[Au(TPPTS)₃] that allows for physical crosslinking and formation of polyelectrolyte complex phosphorescent nanoparticles. This mechanism is similar to an ionic gelation mechanism proposed by Bhumakar et al.¹¹⁰ and an opposite charge mechanism is proposed by Parkin et al.¹¹¹

Chitosan (CS) with a pKa of 6.5 is polycationic, positively charged polysaccharide when dissolved in acid presents —NH3⁺ sites, AuP dissolved in water consists of 9 anionic sulfonate groups available from each planar AuP molecule. This can favor more crosslinking points compared to well known linear tripolyphosphate molecules with only 3 phosphate groups per tripolyphosphate molecule or 2.3 sulfate groups per glucosyl residue in dextran sulfate polymer that are very well documented physical crosslinking agents for formation of CS nanoparticles^{110,111}, though phosphorescence is an extra added advantage using proposed transition/noble metal based phosphors.

Formation of polymeric phosphorescent polyelectrolyte complex particles were studied using dynamic light scattering. At fixed pH and fixed wt. % of CS the number of free amine groups are also fixed that makes Na₈[Au(TPPTS)₃] concentration a deciding factor in tuning size of the particles. Light scattering data in FIG. 18A shows initially no light scattering signal from CS polymer itself solubilized in 1% acetic acid (˜pH-4.5), but on addition of Na₈[Au(TPPTS)₃] interactions of SO3⁻ with —NH3⁺ groups result in formation of polyelectrolyte complex nanoparticles confirmed from strong light scattering signal and colorless polymer solution turning opalescent (FIG. 18C). As phosphor concentration increases chitosan matrix becomes more compact due to increased ionic interactions between CS and Na₈[Au(TPPTS)₃] resulting in smaller size particles. (FIG. 18A). Nevertheless above 0.01 molar concentration, CS—AuP polyelectrolyte complex precipitates out of aqueous medium as solubility of CS is completely dependent on availability of free —NH3⁺ groups in solution. pH sensitivity of these polyelectrolyte complex particles as demonstrated herein shows that swelling and deswelling behavior at different pH due to intrinsic pH sensitive property of CS itself. Film forming ability of CS has been utilized to^{112,113} quickly demonstrate a facile way for formation of AuP loaded chitosan films that are stable for more than a year retaining strong emission along with microseconds lifetime (FIG. 38) that can be useful for optical and biological applications.

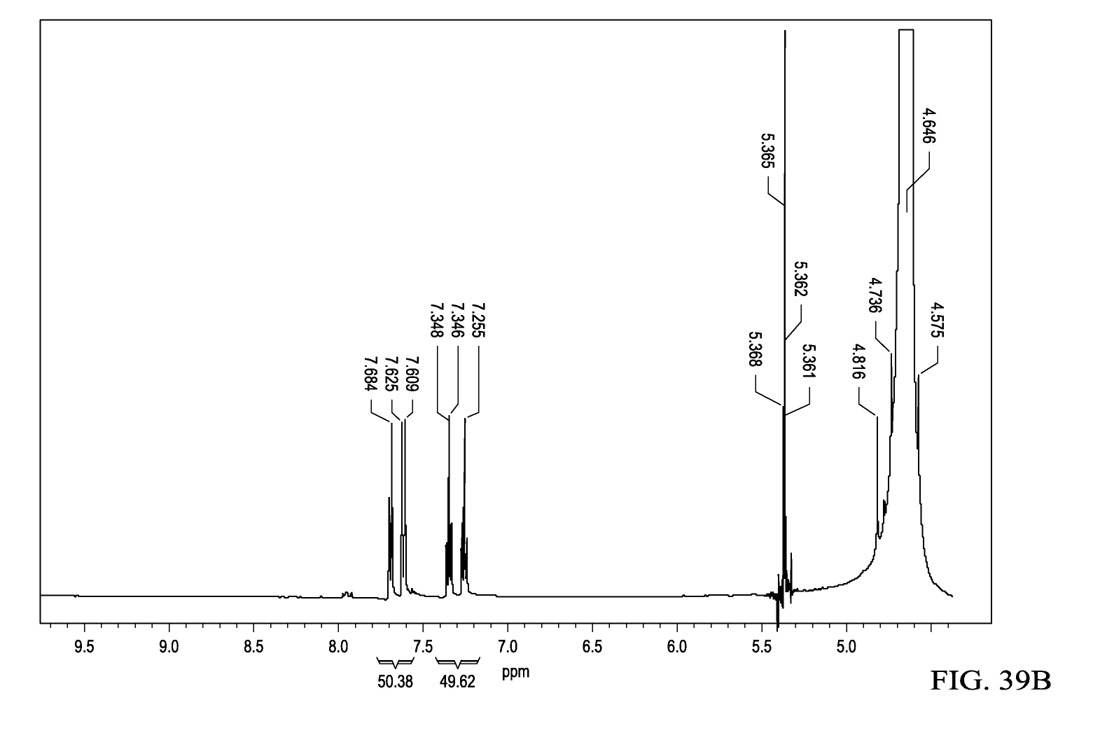

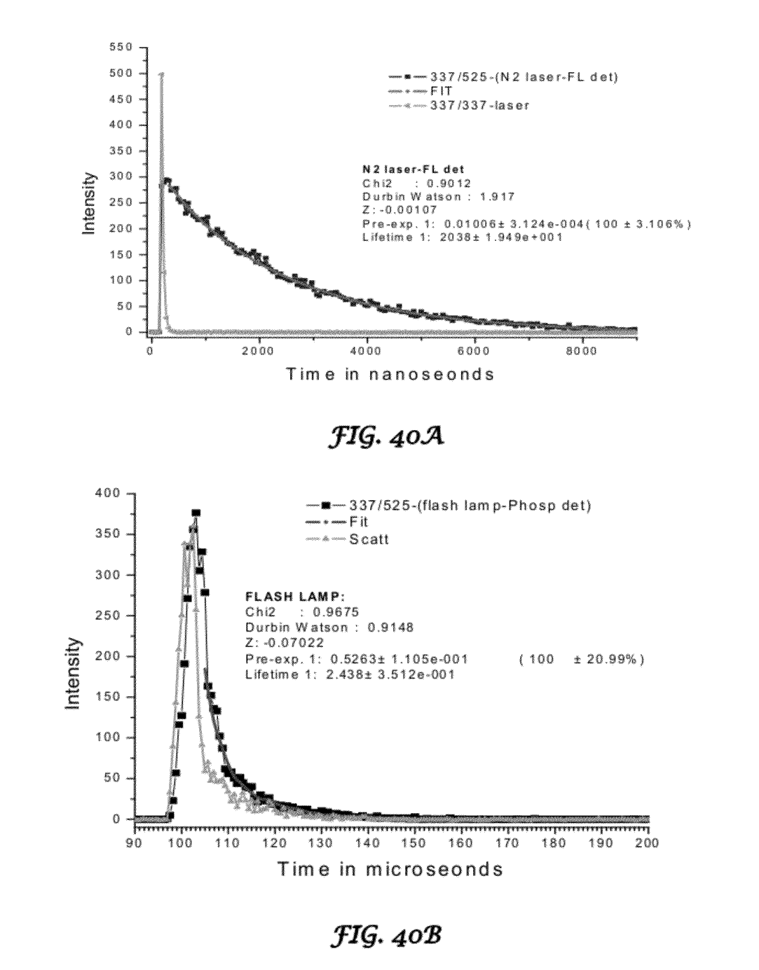

Through FTIR analysis of CS, Na₈[Au(TPPTS)₃] and CS—Na₈[Au(TPPTS)₃] nanoparticles shown in FIGS. 39A and 39B, the interactions between CS and AuP are elucidated. Table 3 lists out characteristic peaks which are in close agreement with observations in literature¹¹⁴⁻¹¹⁷. The spectrum of CS—Na₈[Au(TPPTS)₃] complex did not present any new peaks except for narrowing of peaks followed by increased intensity that clearly indicates ionic interactions between CS and AuP and does not change actual chemical structure of either, even after dialysis, and all workout techniques that establishes retaining of Na₈[Au(TPPTS)₃] in CS matrix and polyelectrolyte interactions between CS and Na₈[Au(TPPTS)₃].

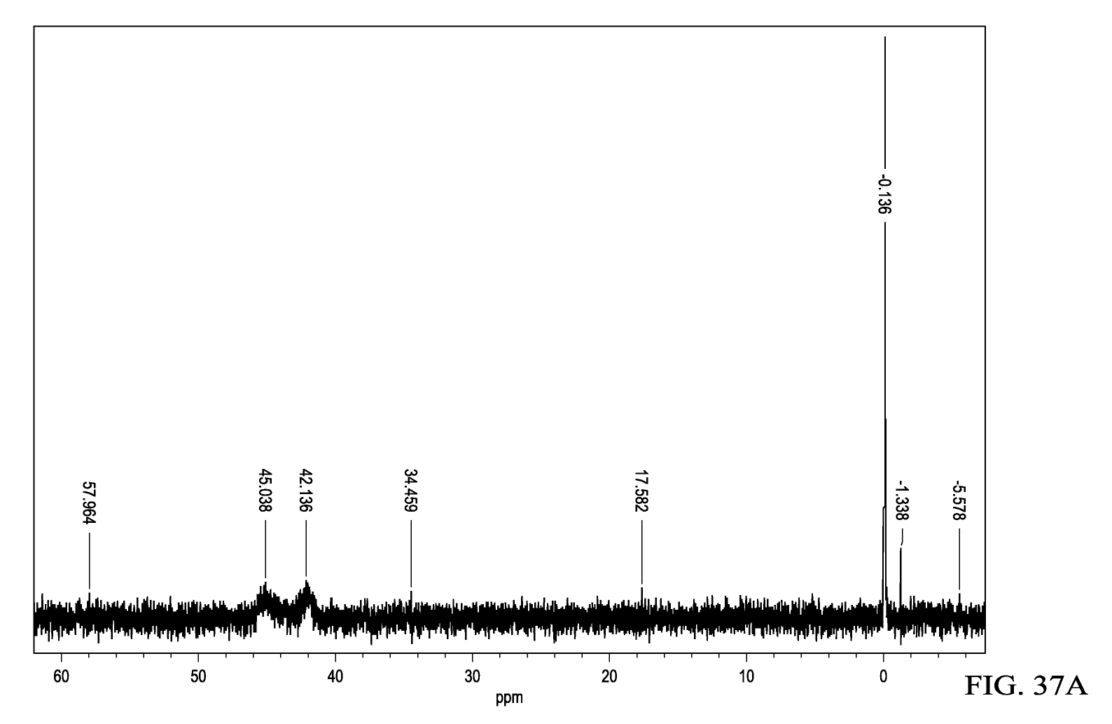

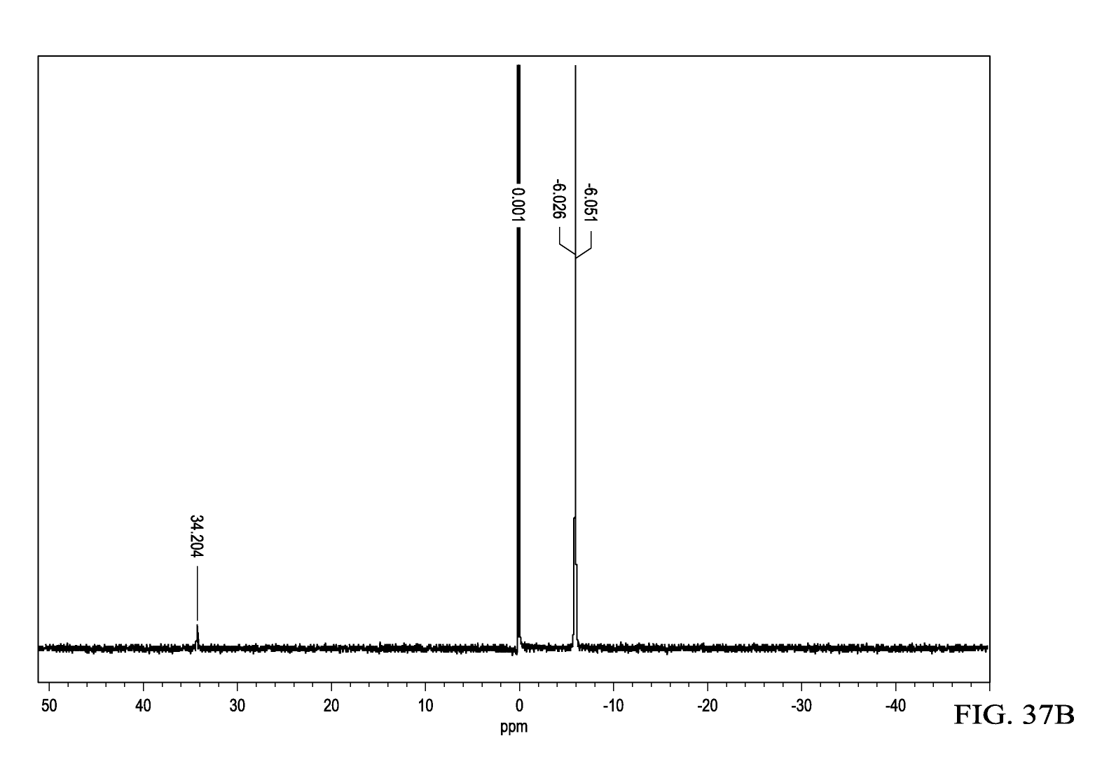

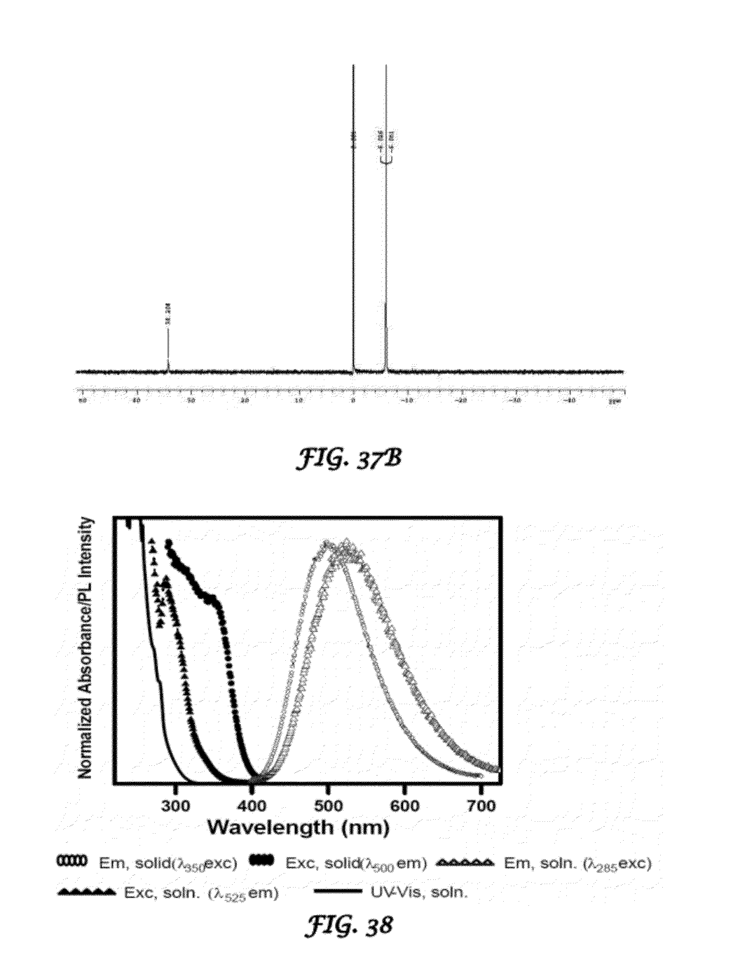

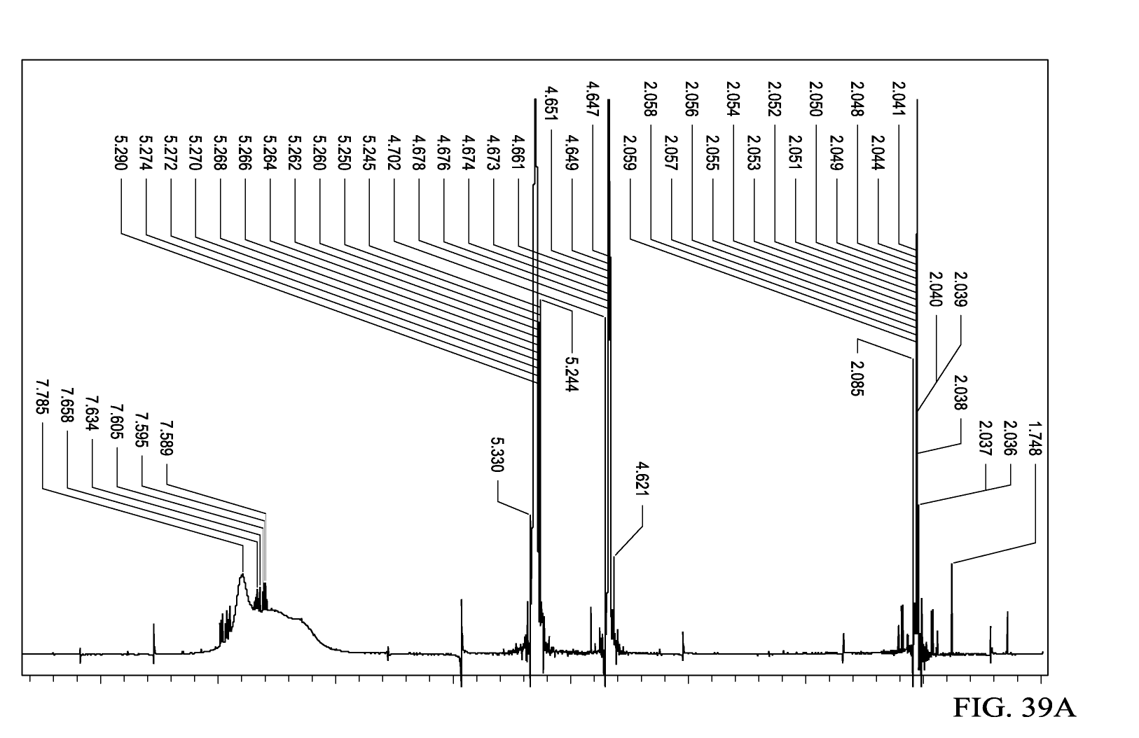

TABLE 3
Comparison of selected characterization parameters of
Na₈[Au(TPPTS)₃] samples prepared in the present
invention vs. reported literature data^{65, 66}.

| Property | Present Invention | Literature results^{65, 66} |
|---|---|---|
| Uv-Vis | 271 and 280 nm | 270 nm and 280 nm |
| solution | | |
| ³¹P{¹H} NMR | 45.04 & 42.14 ppm | 45.5 and 43.5 ppm (both broad) |
| (in D₂O) | (both broad) | (Free TPPTS: −5.2 ppm; 34.5 |
| | (Free TPPTS: −6.02 | ppm TPPTS oxide impurity) |
| | ppm; 34.45 ppm | |
| | TPPTS oxide impurity, | |
| | FIGS. 37A and 37B). | |
| PL, soln. (RT) | Uncorr.: 296 nm exc.; | 293 nm exc.; 513 nm em. |
| | 510 nm em. Corr.: 285 | (broad) |
| | nm exc.; 525 nm em. | |
| τ, soln. (RT) | 2.04 ± 0.02 μs (FIGS. | 1.9 μs (fitting error not given) |
| | 40A and 40B) | |

After 2 days of dialysis, CS—AuP polyelectrolyte complex solution exhibited very similar photoluminescence properties FIGS. 18B and 18C as AuP aqueous solution itself with characteristic broad emission at ˜530 nm signifying the retention of AuP in CS—AuP polyelectrolyte nanoparticle complex in its native form which is desired as most of the conventional organic dyes are sensitive to changes in microenvironment⁵⁹.

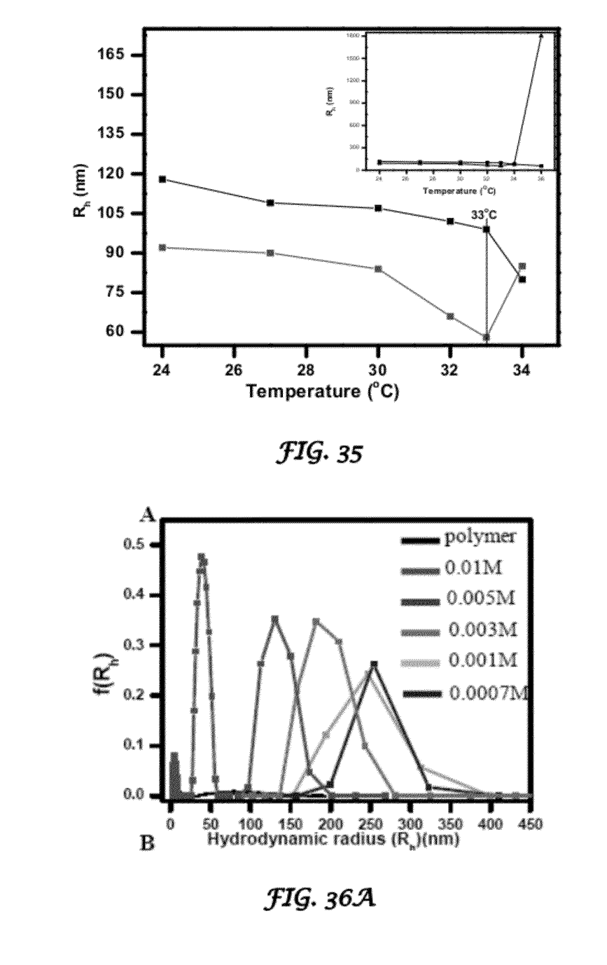

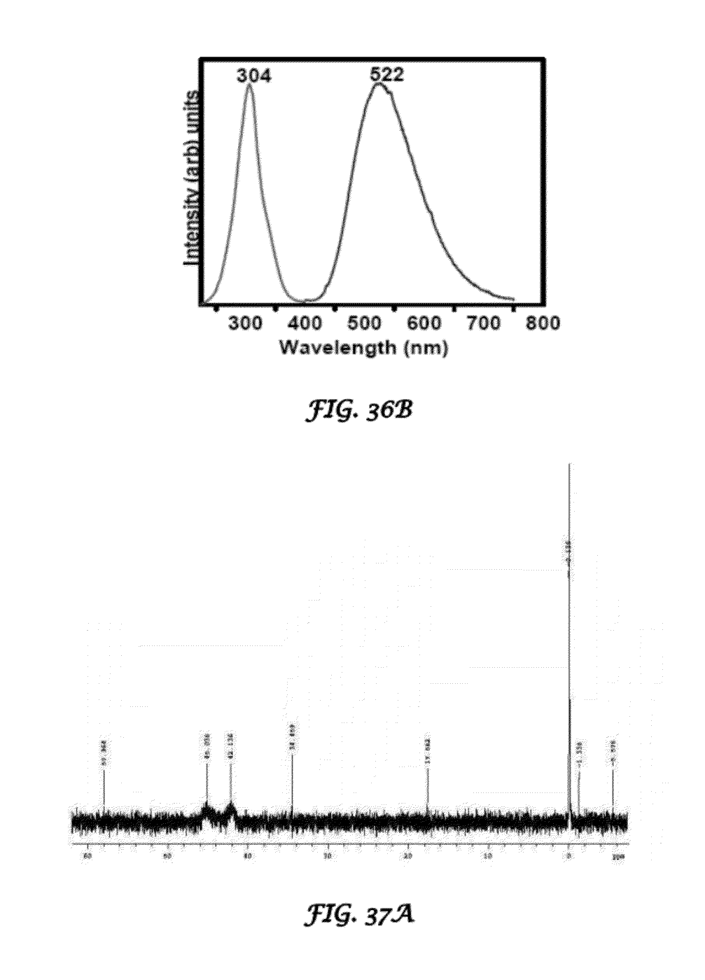

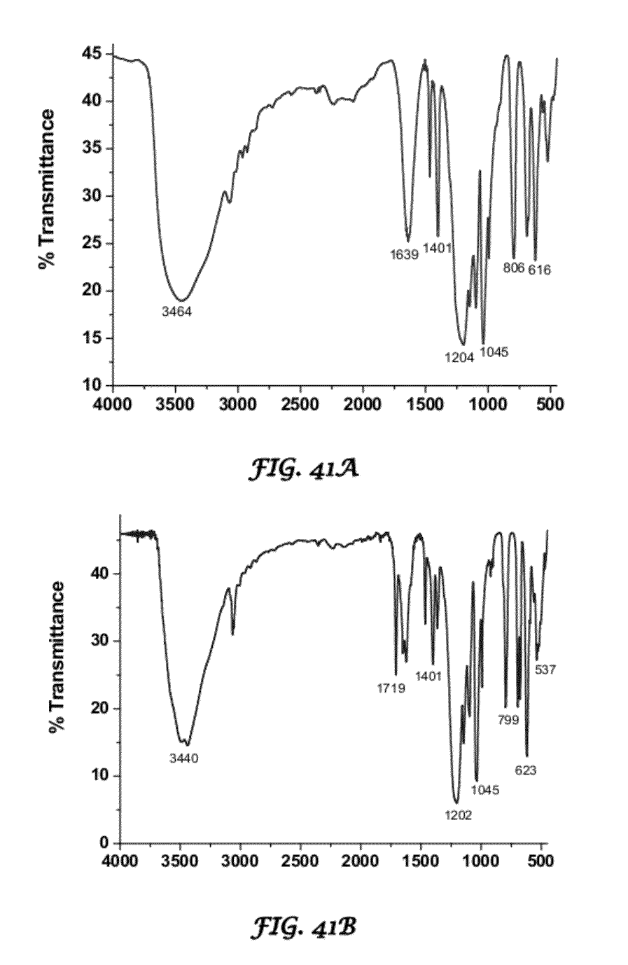

With the above results the inventors confirm development of a new gold based phosphorescent physical crosslinker with the ability to form phosphorescent CSNPS. It is expected that the method of forming microgels as described herein using AuP as a physical crosslinker can be applied to other positively charged polymer chains as well. As a demonstration, the inventors applied the similar procedures to PNIPAM-RCONH₂ chains. FIG. 36A shows formation and changes in hydrodynamic radius of the PNIPAM-R—CONH₂/AuP microgels with respective to AuP concentration. Sharp decrease in (Rₕ) from 252 nm to 36 nm with increased scattered light intensity on increasing the concentration of AuP indicates clear aggregation of PNIPA-RCONH₂ co-polymer chains at room temperature in presence of AuP due to strong ionic interactions. Note that the luminescence of the so formed aggregates also retained similar photophysical properties as AuP in aqueous solutions exhibiting bright metal centered emission ˜528 nm (FIG. 36B). Here positively charged amine groups of co-polymer NIPA-N-(3-aminopropyl)methacrylamide interact with the sulfonate groups of AuP, resulting in polymer aggregates in solution with altered hydrodynamic radius (Rₕ) above and below the phase transition temperature of PNIPAM polymer (˜33° C.). Thus, the resulting polymer aggregates exhibit continuous increase in size with increase in temperature (FIGS. 41A and 41B) due to electrostatic interactions between AuP and the polymer that overcome repulsions among polymer particles. These results are very similar to data observed from PNIPAM-co-allylamine microgel aggregates due to inverse thermo reversible physical gelation (Table 2). Both these temperature dependent light scattering data confirm that there are strong ionic interactions between positively charged amine groups and polyanionic sulfonate groups below and above phase transition temperature either in the form of PNIPAM microgel or PNIPA polymer chain resulting in formation of thermo reversible aggregates.

The work presented herein provides the first reported example of a transition metal based phosphors ability to induce inverse thermoreversible physical gelation in PNIPAM-co-allylamine microgels with retained photoluminescent properties in both sol and gel forms along with reporting a straight forward approach in syntheses of phosphorescent chitosan and PNIPA polymer based nanoparticles by simple ionic interactions between positively charged amine groups of the polymer and anionic sulfonate groups of the Na₈[Au(TPPTS)₃]. Retained photoluminescence properties in microgels on gelation and also in PNIPA and chitosan complex nanoparticles signifies stability of AuP in presence of different matrix systems crucial for utilizing such systems for labeling and other biological studies. Concentrations of PNIPAM-co-allylamine microgels, AuP are observed to be determining factors for tuning inverse thermoreversible gelation, while size of phosphorescent polymeric nanoparticles are shown tunable with changes in concentration of AuP alone. Light scattering and PL data clearly demonstrates formation and tunability of both chitosan and PNIPA-RCONH₂ polymeric particles with preserved thermosensitive and pH sensitive properties revealing the AuP physical crosslinking capability while un-inhibiting intrinsic properties of polymers and AuP as well. The method can be further engaged with other possible biocompatible and thermosensitive polymer systems to form phosphorescent nanoparticles useful for different biomedical applications.

A skilled artisan will recognize that it is possible to make this or a modification of this invention possible by varying metal based phosphor from gold/platinum/Cu/Ag to other transition metals like Ruthenium, Rhodium, Iridium or Osmium, which are well studied transition metals for making luminescent metal compounds. The enhancement property can be attained in other luminescent systems like metal oxide nanoparticles or quantum dots which can have significant applications in biological and materials applications. PNIPAM microgels can be varied with PEG or latex based particles which are considered to be more biologically benign and already commercially available in the market. Varying structure of anionic transition metal based complexes similar to biologically related molecules like Auranofin, Solganol, C is-platin which have both medical importance and photoluminescence properties. Later dope them into similar gels/polymers for enhancing their biological applications. Biologically benign chitosan polymer can be varied with negatively charged polymers like alginic acid, polyacrylic acid or polylactic acid which already have tremendous applications in different fields.

Loading of transition noble metal based (Au or Pt) phosphorescent systems into different PNIPAM based microgels and biological benign or thermosensitive polymers for enhancing the stability of metal based phosphors and also to attain distinctive properties from these hybrid systems can be employed for sensing and biological imaging applications as discussed in the examples herein below.

### Example I

### Application as Antitumor Therapeutic Agents

Clinical successes of cisplatin, many platinum and non-platinum based metallodrugs are currently investigated as antitumor agents. Gold(I) based drugs have been shown to have several potential biological applications due to low toxicity of the gold. Significant number of gold(III) and gold(I) compounds, with highly different chemical structures, have been against various human cancer cell lines for their activity⁸⁹. The noble (Au/Pt) based phosphorescent molecules presented in this invention can also be studied for similar activities with additional advantage of phosphorescence which would make these molecules easily imageable and traceable in living cells.

Thermo-responsive polymeric carriers that have gelation properties close to body temperature could be designed to carry a soluble anticancer drug that would become insoluble and accumulate in locally heated regions when injected in vivo. This could be better monitored when the polymeric system has gelation temperature close to body temperature with added advantage of emission for better monitoring. Such polymeric systems can be employed for local hyperthermia treatments using clinically approved anticancer/antitumor drugs⁹⁰.

### Example II

### Imaging Applications

Advances in instrumentation technology have made luminescence spectroscopy a very versatile and powerful tool for the characterization of polymers and hydrogel systems in presence of fluorescent dyes or quantum dots. In order to track the gel swelling behavior or to monitor the location of gel particles in cells⁵³. But most of fluorescent dyes and quantum dots come with their own advantages and disadvantages. Like short nanosecond lifetimes of fluorescent labels that may coincide with any biological background emission (auto-fluorescence), self-quenching effects, high photo-bleaching, and weak fluorescence intensity at physiological pH where as quantum dots come with their own disadvantages like necessity of surface modification, entrapment within polymer systems for solubility reasons. By doping the transition metal based phosphors into different forms of microgels and biological benign polymers the luminescent molecules can be easily stabilized by the same method as the process is simple and easy. Wide range of polymers or gels can be selected depending on nature of luminescent molecule as loading is only determined based on electrostatic interactions only⁵⁴⁻⁶⁴.

Photoluminescence emission enhancement reported here, in presence of PNIPAM microgels can be widely used for overcoming the disadvantages of different fluorescent dyes or quantum dots for imaging purposes as similar to PL emission enhancement noticed in presence of micelles or metallic nanoparticles^{59-60, 62}. This scattering based emission enhancement mechanism can be applied to detection of tumors from regular cells as tumor cells are reported to have more scattering compared to healthy cells due to irregular growth of cells⁵⁹. This scattering based enhancement can also be used for materials applications⁵². Usage of imaging molecules quantity in biological systems can be minimized if scattering enhancement mechanism can be applied to imaging agents which can be toxic at high concentrations.

Fluorescent nanoparticles are widely used as tracers in many applications ranging from immuno and genetic fluorescence detection, neuroscience and cell labeling in in vitro and in vivo. It has been widely believed and observed that imaging agents trapped within particles avoid photostability and quenching caused due to different complex reasons in biological fluids⁹¹.

Micro and nanospheres which are considered to be popular tools for bioanalysis suffer from intrinsic background fluorescence problem. The background fluorescence emission is shown to be eliminated by two common approaches, on one hand using NIR dyes or using phosphorescent dyes with long decay times where in the background emission can be eliminated by measurement arrangement or time based analysis⁹².

Proteins and polymers are routinely tagged by organic dyes like FITC to study drug release and other properties of microspheres and polymers⁹³. Luminescent nanoparticles are routinely synthesized either by incorporation of organic dyes or quantum dots but suffer from many limitations. Developments of nanoparticle based imaging agents have made it possible to overcome some of these challenges. Luminescent nanoparticles can be conjugated with ligands to target them to a molecular marker of interest, Furthermore, pH- and temperature-sensitive nanoparticles can be used to visualize regional differences in these parameters with modern imaging techniques Molecular imaging enables the non-invasive assessment of biological and biochemical processes. Such technologies therefore have the potential to enhance our understanding of disease and drug activity during preclinical and clinical drug development. Crosslinking reagents that contain a fluorescent moiety are relatively limited with few organic homo- and hetero-bifunctional and trifunctional crosslinkers like dibromobimane which are particularly developed to bring two or more biological molecules together. Only a few examples of phosphorescent materials with crosslinking capabilities are known, mainly/only from Ruthenium/Rhenium/Iridium transition metal complexes acting as crosslinking agents or molecular probes⁹⁴.

Phosphorescent Chitosan nanoparticles outlined in this invention can have considerable advantages and can be employed as perfect imaging agents because of their easy method of preparation and stability compared to regular fluorescent dye doped nanoparticles⁹⁵.

Crosslinked chitosan gels are regularly formed by covalent crosslinking of chitosan like aldehyde, epoxides, cyanates, and other crosslinking charged ions or molecules. A transition metal based phosphor as crosslinking moiety for formation of chitosan nanoparticles has never been achieved. This property of anionic noble (Au/Pt) transition metal based phosphors can be widely used for formation of luminescent polymeric nanoparticles from a wide variety of positively charged polymers⁹⁶.

### Example III

### Environmental Monitoring

Reliable monitoring of volatile organic compounds (VOCs) has significant importance in environment and for public safety. Temperature and pH dependent photoluminescence enhancement detailed in this invention can be used for screening such volatile organic compounds or detection of carcinogenic agents or efficient detection of biological molecules in biological fluids under physiological conditions.

### Example IV

### Small Molecule Detection

As photoluminescence emission enhancement mechanism described in the invention works its best under very low concentrations of metal based phosphors, it can become an attractive alternative compared to Raman scattering detection of small molecules under low concentrations⁶¹.

### Example V

### Live Cell Imaging

Temperature and pH dependent photoluminescence emission enhancement can allow imaging of live cells under suitable physiological conditions without much hindrance due to autofluorescence or self bleaching¹⁰.

### Example VI

### Injectable Scaffolds and Drug Delivery Devices

Microphase separation in aqueous polymer systems can lead complex rheological changes, thermo-thickening polymer systems which particularly exhibit increase in viscosity on heating are shown to have special pharmaceutical and biomedical applications. Luminescence rigidochromism demonstrated in presence of soft gels can be made useful for detection of phase changes in different polymers or gel systems by employing photoluminescence techniques. Polymer solutions with thermoreversible gelation property are highly sought after because they are used as injectable scaffold for delivering medicine or encapsulating cells^{4, 7-9}. Though a good number of photoluminescent sol-gel probes⁷⁶ are reported but never before in aqueous soft matter systems like PNIPAM hydrogels from gold/Pt based luminophores, can be employed for detecting phase transformations in polymers and gels in presence of different chromophores⁹⁷, can further be utilized to detect changes in rigidity and structural changes of polymer/gel matrixes in presence of similar gold based/other transition metal based luminophores that exhibit rigidochromism are capable of acting as gelating agents especially in soft materials.

### Example VII

### Chemical and Biological Recognition

Cyclic trinuclear systems involving aromatic rings are shown to process key roles in chemical and biological recognition. Several examples of organometallic compounds employed in medicine as drugs or diagnosis containing one or more metal atoms are shown to play different roles: Like acting as Lewis-acid binding sites, sensitive units carrying that can increase strength of electrostatic interactions. Gold-gold interactions that results in unusual emissive tuning behavior in cyclic trinuclear gold systems either due to aurophilically bonded dimer-of-trimer units or where luminescence color changes are noticed due to doping/dedoping of metal ions like Ag⁺ as well documented for their interesting applications in sensors and displays. But all this work is restricted to organic medium due to solubility and stability concerns of cyclic trimer systems in an aqueous medium. Stabilizing these systems in presence of polymers as described in this invention can open new route is for stabilizing these very significant physical and chemical sensitive phosphorescent molecules in aqueous medium opening wide scope of applications in biological sensing and imaging^{89,98}.

Phosphorescent heavy metal complexes are long known for a multitude of photonic applications and as biological labeling agents⁹³. But recently these heavy metal complexes have attracted considerable interest as chemosensors or heavy metal ion sensors due to strong photophysical properties. Such as a consequential shift in the emission and excitation wavelength, significant stoke shifts and measurable changes in lifetimes due to change in local environment are better off compared to regular organic luminophore. Until now only few classes of transition compounds are explored as chemosensors. Like platinum (II) complexes, rhenium (I), Ruthenium (II) and Iridium (III) complexes are successfully explored. But most of these compounds have poor solubility or stability issues in aqueous medium. So stabilizing similar anionic transition metal based phosphors systems in polymer based aqueous medium can play an important role in development of highly selective chemosensors for the detection of toxic transition heavy metal ions like Mercury/Lead/Thallium in different biological systems and from ground water⁹⁹⁻¹⁰⁰.

A skilled artisan will recognize that there can be other applications of the present invention and the present invention can be adapted or modified to confer additional characteristic and advantages to solve problems not addressed herein. The anionic transition noble metal (Au/Pt) based phosphors described in this invention can themselves or after entrapping in polymeric nanoparticles be tested for antitumor activities on various cell lines, The film forming ability of chitosan and other polymers can be used for formation of luminescent films after doping them with above-mentioned anionic phosphors which could be used as volatile organic sensors or pH sensors.

The technology, compositions, and methods disclosed herein are superior and offer advantages over existing technologies and the prior art. The performance of metal based phosphors either for detection/sensing or imaging depends on stability in aqueous and biological medium. However, most of the transition metal based phosphors already known in literature suffer from serious backdrop of solubility or stablity only in organic solvents. Only few stable, water soluble transition metal based phosphors are known till date. So this invention not only focuses on water soluble anionic noble metal (Au/Pt) based transition phosphorescent systems but also shows a way to stabilize them and enhance their optical properties in presence of soft gels and polymers which mimic biological environment. As these anionic phosphors are solely made from either Gold, Platinum or a combination of both such systems are highly stable and less cytotoxic compared to other transition metal based phosphorescent systems already known.

Photoluminescence enhancement reported until now is only restricted to either from micelles or in presence of metallic nanoparticles mostly under room temperature conditions only^{59,62}. But photoluminescence enhancement reported here has wide applications being novel in presence of PNIPAM gels and also being expressed in different pH and temperature conditions that can be applied to many other luminescence systems. This invention discloses a method of photoluminescence enhancement that can help detection of molecules in very low concentrations similar to Raman scattering. PL enhancements from such low concentrations of phosphors either by micelle or nanoparticle was previously unknown.

This invention discloses a novel previously unknown method using an anionic noble (Au/Pt) transition metal based phosphor as gelating agent in presence of positively charged PNIPAM gels. As most of gelation/aggregation mechanisms reported in such polymers or gels is due to presence of oppositely charged polymers or in presence of high concentration of salts like NaNO₃ or CaCl₂¹⁰¹.

Synthesis of luminescent chitosan or polymeric nanoparticles generally involves two steps. The first step is formation of polymeric nanoparticles by using various kinds of physical or chemical crosslinkers, then in the second step luminescence is attained by doping or crosslinking the formed nanoparticles with organic dyes or quantum dots^{102,55}. But this invention discloses a single step method for formation of pH/temperature sensitive polymeric particles with wide ranges in size in a single step method by taking advantage of anionic functional group moieties. This can improve biological applications of probing and imaging aspects of these luminescent nanoparticles as the crosslinker itself is a luminescent agent.

Though phosphorescent transition metal based phosphors for heavy metal ion sensing are well known, but most of them are synthesized and stabilized in organic medium that restricts their applications in biological or aqueous medium. The stabilization mechanism disclosed in this invention takes advantage of water solubility and stability of anionic cyclic trinuclear trimer systems in presence of polymer backbone for heavy metal ion sensing applications in biological medium under physiological conditions.

It is contemplated that any embodiment discussed in this specification can be implemented with respect to any method, kit, reagent, or composition of the invention, and vice versa. Furthermore, compositions of the invention can be used to achieve methods of the invention.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, MB, BBC, AAABCCCC, CBBAAA, CABABB, and so forth. The skilled artisan will understand that typically there is no limit on the number of items or terms in any combination, unless otherwise apparent from the context.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

### REFERENCES

U.S. Pat. No. 6,207,077: Luminescent Gel Coats and Moldable Resins.
WIPO Patent Publication No. WO/2003/102109: Phosphorescent and Luminescent Conjugated Polymers and their use in Electroluminescent Assemblies.
WIPO Patent Publication No. WO/2005/021678: Phosphorescent Polymer Compound and Organic Light Emitting Device using the Same.
1. Huang, X.; Lu, T.; Chen, M.; Guo, X. J. Poly. Res. 2008, 15, 41.
2. McCoy, P. C. Chem. Mater. 2006, 18, 4336.
3. Yan, B.; Xiang, Y.; Wang, Q.; Liu, Y. J. Fluoresc. 2006, 16, 723.
4. F. Cellesi, N. Tirelli, J. Mat. Sci: Mat in Med. 2005, 16, 559.
5. A. Hayashi, S. C. Oh, Agri. Biol. Chem. 1983, 47, 1716.
6. B. Jeong, S. W. Kim, Y. H. Bae, Adv. Drug. Deli. Rev. 2002, 54, 37.
7. B. Jeong, Y. H. Bae, S. W. Kim, Macromolecules. 1999, 32, 7064.
8. D. S. Lee, M. S. Shim, S. W. Kim, H. Lee, I. Park, T. Chang, Macro. Rapid. Commun. 2001, 22, 587.
9. Z. Hu, X. Xia, Adv. Mat. 2004, 16, 4, 305.
10. Kam-Wing Lo, K.; Chun-Ming, D.; Chi-Keung, C. Organometallics 2001, 20, 4999.
11. Chem. Eur. J. 2003, 9(2), 475.
12. Nayak, S.; Lyon, A. L. Angew. Chem. Int. Ed. 2005, 44, 7686.
13. Siegel, R. A.; Firestone, B. A. Macromolecules. 1988, 21, 3254.
14. Hirotsu, S.; Hirokawa, Y.; Tanaka, T. J. Chem. Phys. 1987, 87, 1392.
15. Wang, J.; Gan, D.; Lyon, A. L.; El-Sayed, M. A. J. Am. Chem. Soc. 2001, 123, 11284.
16. Suzuki, D.; Tsuji, S.; Kawaguchi, H. J. Am. Chem. Soc. 2007, 129, 8088.
17. Keerl, M.; Pedersen, J. S.; Richtering, W. J. Am. Chem. Soc. 2009, 131, 3093.
18. Kwon, I. C.; Bae, H. Y.; Kim, W. S, Nature. 1991, 354, 291.
19. Osada, Y.; Okuzaki, H.; Hori, H. Nature. 1992, 355, 242.
20. Chen, J.; Yang, L.; Ding, G.; Lie, J.; Huang, G. Macromol. Symp. 2005, 225, 71.
21. Kondo, A.; Fukuda, H. J. Ferm. Bioeng. 1997, 84, 337.
22. Jones, C. D.; Serpe, M. J.; Schroeder, L.; Lyon, L. A. J. Am. Chem. Soc. 2003, 125, 5292.
23. Gorelikov, I.; Field, L. M.; Kumacheva, E. J. Am. Chem. Soc. 2004, 126, 15938.
24. Garcia, A. Marquez, M.; Cai, T.; Rosario, R.; Hu, Z. B.; Gust, D.; Hayes, M.; Vail, S. A.; Park, C. Langmuir. 2007, 23, 224.
25. Pelton, R. Adv. Coll. Inter. Sci. 2000, 85, 1.
26. Peppas, N. A. Hydrogels in medicine and pharmacy, CRC Press, 1986.
27. Hoffman, A. S. Adv. Drug Delivery Rev. 2002, 43, 3.
28. Hu, Z.; Gao, J. Langmuir. 2002, 18, 1360.
29. Gao, J.; Frisken, B. Langmuir. 2005, 21, 545.
30. Hu, Z.; Huang, G. Angew. Chem. Int. Ed. 2003, 42, 4799.
31. Xia, X.; Hu, Z.; Marquez, M. J. Contr. Drug Rel. 2005, 103, 21.
32. Tang, S. J.; Hu, Z. B.; Zhou, B.; Cheng, Z. D.; Wu, J. Z.; Marquez, M. Macromolecules. 2007, 40, 9544.
33. Doherty, P.; Benedek, G. B. J. Chem. Phys. 1974, 61, 5426.
34. Qui, Y.; Park, K. Adv. Drug Delivery Rev. 2001, 53, 321.
35. Bouhadir, H. K.; Alsberg, E.; Mooney, J. D. Biomater. 2001, 22, 2625.
36. Moretto, A.; Tesolin, L.; Marsilio, F.; Berna, M.; Veronese, F. M. IL FARMCO. 2004, 59, 1.
37. Huang, G.; Hu, Z.; John, V.; Ponder, B. C.; Moro, D. J. Contr. Drug Rel. 2004, 94, 303.
38. Petrak, K. Drug Discov. Today. 2005, 23/24, 1667.
39. Reese, C. E.; Mikhonin, A. V.; Kamenjicki, M.; Tikhonov, A.; Asher, S. A. J. Am. Chem. Soc. 2004, 126, 1493.
40. Kim, J.; Serpe, J. M.; Lyon, A. L. J. Am. Chem. Soc. 2004, 126, 9512.
41. Mathew, W. H.; Salley, O, S.; Peterson, D. W.; Klein, D. M. Biotech. Prog. 1993, 9, 510.
42. Apkarian, P. R.; Wright, R. E.; Seredyuk, A. V.; Eustis, S.; Lyon, A. L.; Conticello, P. V.; Menger, M. F. Microsc. Microanal. 2003, 9, 286.
43. Ozkul, N. A.; Khare, A. R. Adv. Drug. Deliv. Rev. 1993, 11, 1.
44. Kim, J.; Lyon, A. L.; Nayak, S. J. Am. Chem. Soc. 2005, 127, 9588.
45. Kiousis, R. D.; Smith, F. D.; Kofinas, P. J. Appl. Poly. Sci. 2001, 80, 2073.
46. Kofinas, P.; Kiousis, R. D. Environ. Sci. Technol, 2003, 37, 423.
47. Stile, A. R.; Burghardt, R. W.; Healy, K. E. Macromolecules. 1999, 32, 7370.
48. Boelen, J. H. E.; Hooy-Corstjens, S. J. C.; Bulstra, k. S.; Koole, H. L.; Rhijn, W. L. Biomater. 2005, 26, 6674.
49. Byrne, E. M.; Park, K.; Peppas, N. A. Adv. Drug. Delivery Rev. 2002, 54, 149.
50. Gao, J.; Frisken, J. B. Langmuir. 2003, 19, 5217.
51. D'Andrade, B. W.; Forrest, S. R. Adv. Mater. 2004, 16, 1585.
52. Reineke, S.; Lindner, F.; Schwartz, G.; Seidler, N.; Walzer, K.; Lüssem, B.; Leo, K. Nature. 2009, 459, 234.
53. Rangarajan, B.; Coons, L. S.; Scranton, A. B. Biomater. 1996, 17, 649.
54. Wu, Z.; Gong, S.; Li, C.; Zhang, Z.; Huang, W.; Meng, L.; Lu, X.; He, Y. Eur. Polym. J. 2005, 41, 1985.
55. Gao, H.; Zhao, Y.; Fu, S.; Li, B.; Li, M. Colloid. Polym. Sci. 2002, 280, 653.
56. Das, R. R.; Samal, S.; Choi, S. S.; Geckler, K. Macromol. Rapid Commun. 2001, 22, 850.
57. Gong, Y.; Gao, M.; Wang, D.; Mohwald, H. Chem. Mater. 2005, 17, 2648.
58. Li, J.; Hong, X.; Liu, Y.; Wang, Y.; Li, J. Adv. Mater. 2005, 17, 163.
59. Lakowicz, J. R. Anal. Bio. 2001, 298, 1.
60. Wang, Y. H.; Zhou, J.; Zong, R. L.; Shi, S. K.; Wang, T.; Li, B. Optoelectron. Lett. 2006, 2, 0316.
61. Kneipp, K.; Wang, Y.; Kneipp, H.; Perelman, L. T.; Irving, I.; Dasari, R. R.; Feld, M. S. Phys. Rev. Lett. 1996, 78, 1667.
62. Takeuchi, T. J. Chromatogr. A 1997, 780, 219.
63. Sanz-Medel, A.; Alonso, J. I. G.; Gonzllez, E. B. Analy. Chem. 1985, 57, 1681.
64. Susumu, K.; Medintz, I. L.; Mattoussi, H. Colloidal quantum dots: synthesis, photophysical properties and biofunctionalization strategies. In Inorganic Nanoprobes for Biological Sensing and Imaging; Mattoussi, H.; Cheon, J.; House, A. (Eds.); Norwood: MA, 2008, Chapter 1, pp. 1-26.
65. Forward, M. J.; Assefa, Z.; Fackler, J. P. Jr. J. Am. Chem. Soc. 1995, 117, 9103.
66. Assefa, Z.; Forward, M. J.; Grant, A. T.; Staples, J. R.; Hanson, E. B.; Fackler, J. P. Jr. lnorg. Chim. Acta. 2003, 352, 31.
67. Sinha, P.; Wilson, A. K.; Omary, M. A. J. Am. Chem. Soc. 2005, 127, 12488.
68. Barakat, K. A.; Cundari, T. R.; Omary, M. A. J. Am. Chem. Soc. 2003, 125, 14228.
69. Sinha, P., Phosphorescent Emissions of Coinage Metal-Phosphine Complexes:

Theory and Photophysics, Ph.D. Dissertation, Robert B. Toulouse School of Graduate Studies, University of North Texas, 2009.
70. Wrighton, M.; Morse, D. L. J. Am. Chem. Soc. 1974, 96, 998.
71. Fredericks, S. M.; Luong, J. C.; Wrighton, M. S. J. Am. Chem. Soc. 1979, 101, 7415.
72. Giordano, P. J.; Wrighton, M. S. J. Am. Chem. Soc. 1979, 101, 2888.
73. McKiernan, J.; Pouxviel, J. C.; Dunn, B; Zink, J. I. J. Phys. Chem. 1989, 93, 2129.
74. Dunn, B.; Zink, J. I. Chem. Mater. 1997, 9, 2280.
75. Rickus, J. L.; Chang, P. L.; Tobin, A. L.; Zink, J. I.; Dunn, B. J. Phys. Chem. B. 2004, 108, 9325.
76. Dunn, B; Zink, J. I. Acc. Chem. Res. 2007, 40, 747.
77. Vogler, A.; Kunkley, H. Mater. Chem. Phys. 2008, 109, 506.
78. Mohwald, H.; Wang, D.; Gao, M.; Gong, Y. Chem. Mater. 2005, 17, 2648.
79. MacDonald, B. C.; Lvin, S. J.; Patterson, H. Anal. Chim. Acta 1997, 338, 155.
80. Zhang, C.; Liu, M.-S.; Han, B.; Xing, X.-H. Anal. Biochem. 2009, 390, 197.
81. Lloyd, J. B. F. Tech. Vis. Ultraviol. Spectrom. 1981, 2, 27.
82. Boens, N.; Ameloot, M.; Valeur, B. Springer Series on Fluorescence 2008, 5, 215.
83. Neogi, A.; Garner, B.; Cai, T.; Kim, M.; Hu, Z. B. Soft Mater. 2009, 7, 232.
84. John, S.; Marpu, S.; Li, J.; Omary, M.; Hu, Z.; Fujita, Y.; Neogi, A. J. Nanosci. Nanotech. 2010, 10, 1707.
85. Kishimura, A.; Yamashita, T.; Aida, T. J. Am. Chem. Soc. 2005, 127, 179.
86. Tam, A. Y.-Y.; Wong, K. M.-C.; Yam, V. W.-W. J. Am. Chem. Soc. 2009, 131, 6253.
87. Lawandy, N. M.; Balachandran, R. M.; Gomes, A. S. L.; Sauvain, E. Nature (London) 1994, 368, 436.
88. Tam, F.; Goodrich, G. P.; Johnson, B. R.; Halas, N. J. Nano Lett. 2007, 7, 496.
89. Abdou, H. E., et al. “Structure and properties of gold (I) complexes of interest in biochemical applications” Coordination. Chem. Rev. 2009, 253, 1661.
90. Meyer, D. E.; Shin, B. C.; Kong, G. A.; Dewhirst, M. W.; Chilkoti, A. J. Cont. Drug. Release. 2001, 74, 213.
91. Li, M.; Li, B.; Fu, S.; Zhao, Y.; Gao, H. Coll. Poly. Sci. 2002, 280, 653.
92. Haugland, R. P. fluorescence detection methods including FluoSpheres and ELF technologies (Chapter 6). Handbook of fluorescent probes and research chemicals. Molecular probes Inc.
93. Lo, K. K. “Luminescent transition metal complexes as biological labels and probes” Structure and Bonding. Book chapter, 2007, 123, 205.
94. Lo, K. K.; Ng, D. C.; Chung, C. K. Organometallic. 2001, 20, 4999.
95. Agarwal. P. et al. Adv Drug Delivery Rev. 2010, 62, 2010 42-58.
96. Bodnar, M.; Hartmann, J. F.; Borbely, J. Biomacromolecules. 2005, 6, 2521.
97. Chon, A. K., Chong, Z., Bohlka, M. A. and Tsourkas, A. Anal. Chem. 2008, 90, 7437.
98. T. Aida et al. J. Am. Chem. Soc. 2005, 127, 179.
99. Zhao, Q.; Li, F.; Cao. T.; Li, X.; Jing, H.; Huang. C. Organic Letters, 2007, 9, 23, 4729.
100. Organometallics, 2008, 26, 8, 2077.
101. Gan, T, Zhang, Y, Guan, Y Biomacromolecules. 2009, 10, 1410.
102. Zhang, Y.; Tan, W. B.; Nie, Q. Nanotechnology, 2006, 17, 140.
103. Fackler, J. P., Grant, T. A., Hanson, B. E. Gold Bulletin. 1999, 32, 1, 20.
104. Wing Lo, K. K. Struct Bond, Springer 2006, 123, 205.
105. Lo, K. W., Hui, W. K. Chung, C. K., Tsang, K. H., Ming, D. C., Zhu, N., Cheung, K. K. Cord. Chem. Rev. 2005, 249, 1434.
106. Lo, K. W., Chung, C. K., Zhu, N. Chem. Eur. J. 2003, 9, 2, 475.
107. Benee, L. S., Snowden, M. J., Chowdhry, B. Z. Langmuir. 2002, 18, 6025.
108. Liu, R., Leonardis, P. D., Cellesi, F., Tirelli, N., Saunders, B. R. Langmuir. 2008, 24, 7099.
109. Zhu, P. W., Napper, D. H. Macromolecules. 1999, 32, 2068.
110. Bhumkar, D. R., Pokharkar, V. B. AAPS Pharm Sci Tech. 2006, 7, 2, El.
111. Chen, Y., Mohanraj, V. J., Parkin, J. E. Letters in Peptide Science. 2003, 10, 621.
112. Jiang, H. Liang, J., Grant, J. T., Su, W., Bunning, T. J., Cooper, T. M., Adams, W. W. Macrmol. Chem. Phys. 1997, 198, 1561.
113. Niekraszewicz, A. Fibres & Textiles in Eastern Europe. 2005, 13, 16.
114. Moura, M. R., Aouada, F. A., Mattoso, L. H. C. J. Coll. Int. Sci. 2008, 321, 477.
115. Banerjee, T., Mitra, S., Singh, A. K., Sharma, R. K., Maitra, A. Int. J. Pharm. 2002, 243, 93.
116. Fernandes, A. I. P., Morais, W. A., Santos, A. I. B., D. dos Santos, S., Pavinatto, F. J., Oliveira Jr, O. N., Dantas, T. N. C., Pereira, M. R., Fonseca, J. I. C. Coll. Poly. Sci. 2005, 284, 1.
117. Xian, Z., Jun, L. Min, W. Chinese Science Bulletin. 2008, 53, 9, 1329.
118. Filomena Dos Remedios Pinto, M. A. Sadler, P. J.; J. C. S. Chem. Commun., 1980, 13-15.
119. J. Am. Chem. Soc. 2001, 123, 1613-1624.
120. O'Connor, M. B., Killard, A. J., O'Kennedy, R, Forster, R. J. Smyth, M. R. Jour. Inorg. Bio. Chem, 100, 7, 2006, 1252-1259.

What is claimed is:

1. A stimulus-responsive water soluble hybrid phosphorescent system comprising:
one or more polyanionic or polycationic transition metal based phosphors in the form of a complex, a coordination compound, or combinations thereof; and
a stimulus-responsive matrix comprising a polymer, a hydrogel, a colloid, a microgel, or combinations thereof, wherein the matrix has a charge that is opposite to the polyanionic or polycationic metal based phosphor, wherein the one or more metal based phosphors are linked, attached or entrapped in the matrix to form of one or more luminescent polymeric nano or micro particles in the matrix in the presence or absence of a chemical crosslinking agent.

2. The composition of claim 1, wherein the one or more transition metal based phosphors is coordinated by one or more substituted or unsubstituted phosphine ligands.

3. The composition of claim 1, wherein the one or more transition metal based phosphors is coordinated by one or more azolate ligands including substituted or unsubstituted pyrazolate, triazolate, imidazolate, or combination thereof.

4. The composition of claim 1, wherein the one or more transition metal based phosphors is coordinated by one or more polyimine ligands including substituted or unsubstituted 2,2′-bipyridine, 1,10-phenanthroline, 2,2′:6′,2″-terpyridine, or combination thereof.

5. The composition of claim 1, wherein the one or more transition metal based phosphors have a general formula given by:
[A^{x+}]ₙ₁[(M)ₙ₂(L)ₙ₃]^{y-},
wherein A^{x+} comprises Na⁺, K⁺, Cs⁺, NH₄⁺, R₄N⁺ wherein R is selected from hydrogen or alkyl, Mg⁺², Ca⁺², or combination thereof, M comprises a transition metal selected from the group consisting of gold, silver, copper, platinum, palladium, nickel, ruthenium, osmium, iridium, rhenium, or combination thereof, and at least one L comprises 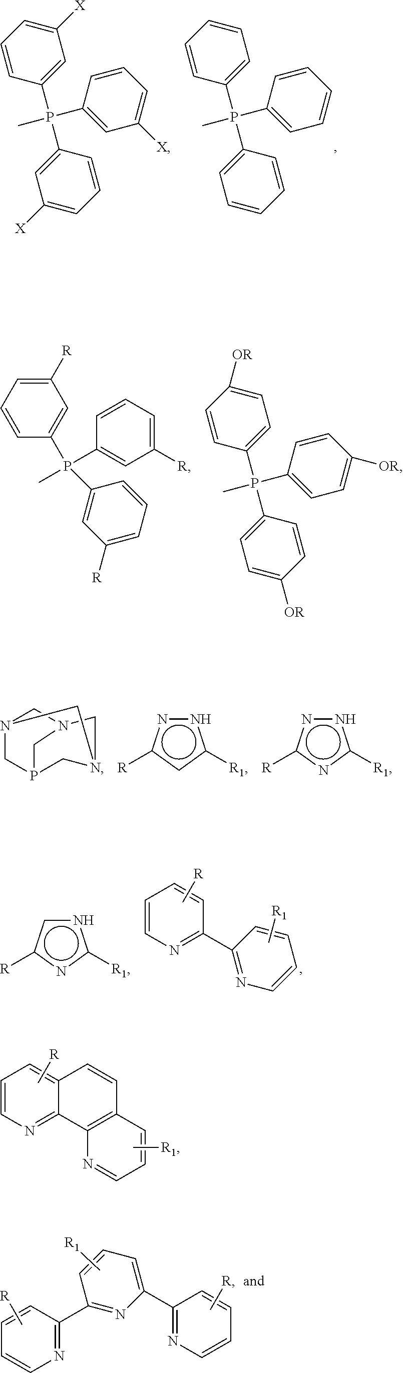
combinations and modifications thereof, wherein R and R₁ are selected from hydrogen, alkyl, alkoxy, aryl, NH₂, NH₃⁺, COOH, COO⁻, SO₃H, SO₃⁻, PO₄H₂, PO₄²⁻, OH, Cl, Br, COOR′, or R′₃N⁺ wherein R′ is selected from hydrogen or alkyl, n₁, n₂, n₃, and y are integer numbers equaling 1 or greater.

6. The composition of claim 1, wherein the one or more transition metal based phosphors have a general formula given by:
[(M)ₙ₁(L)ₙ₂]^{x+}[X^{y-}]ₙ₃,
wherein X^{y-} comprises Cl⁻, Br⁻, I⁻, NO₃⁻, PO₄³⁻, CO₃²⁻, COO⁻, BF₄⁻, PF₆⁻, SO₄²⁻, SO₃²⁻, or combination thereof, M comprises a transition metal selected from the group consisting of gold, silver, copper, platinum, palladium, nickel, ruthenium, osmium, iridium, rhenium, or combination thereof, at least one L comprises 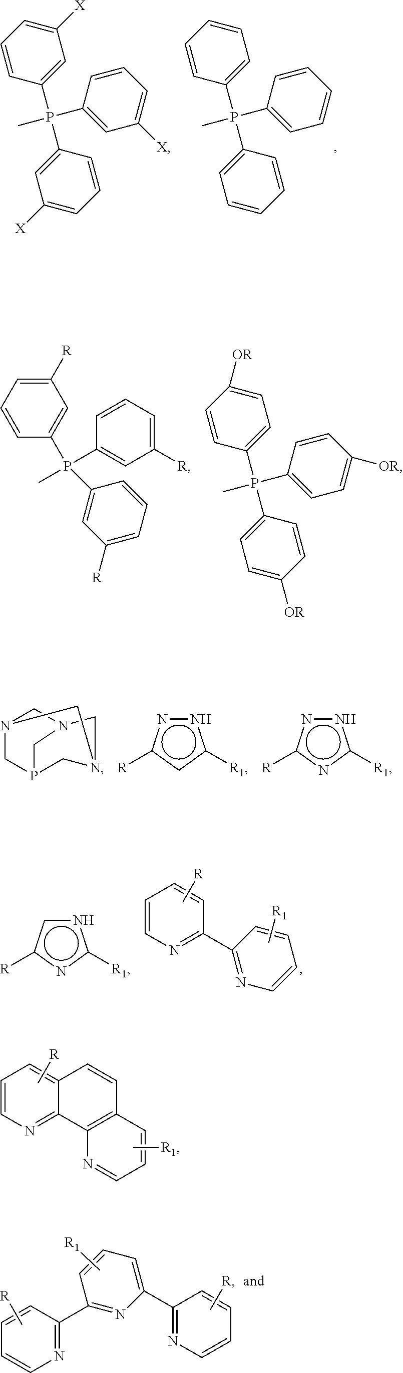
combinations and modifications thereof, wherein R and R₁ are selected from hydrogen, alkyl, alkoxy, aryl, NH₂, NH₃⁺, COOH, COO⁻, SO₃H, SO₃⁻, PO₄H₂, PO₄²⁻, OH, Cl, Br, COOR′, or R′₃N⁺ wherein R′ is selected from hydrogen or alkyl, and n₁, n₂, n₃, and x are integer numbers equaling 1 or greater.

7. The composition of claim 1, wherein the matrix comprises poly-N-isopropylacrylamide (PNIPAM), chemically modified PNIPAM including PNIPAM-co-allylamine and PNIPAM-co-acrylic acid, chitosan, chemically modified chitosan, poly acrylic acid (PAA), polyvinyl alcohol (PVA), alginic acid, PEG, modified PEG, agarose, hydroxy propyl cellulose, methyl methacrylate (MMA), hydroxyethyl methacrylate (HEMA), polystyrene, and poly-hydroxyethyl methacrylate.

8. The composition of claim 1, wherein the composition is responsive to one or more stimuli selected from the group consisting of pH, temperature, electric, magnetic, optical, and environmental stimuli.

9. The composition of claim 1, wherein one of the metal based phosphor has a formula given by [A^{x+}]ₙ₁ tris[tris(3,3′,3″-trisulfonatophenyl)phosphine]aurate(I) wherein [A^{x+}]ₙ₁ comprises [Na⁺]₈, [K⁺]₈, [Cs⁺]₈, [NH₄⁺]₈, [R₄⁺]₈, [Mg⁺²]₄, [Ca⁺²]₄, or combination thereof.

10. The composition of claim 9, wherein the tris[tris(3,3′,3″-trisulfonatophenyl)phosphine]aurate(I) has a structure given by:

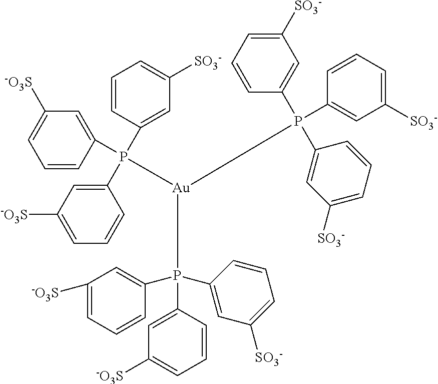

11. The composition of claim 1, wherein the metal based phosphor has a formula given by [Ru(2,2′-Bipyridine)₃](PF₆)₂, [Ru(1,10-Phenanthroline)₃](PF₆)₂, [Os(2,2′-Bipyridine)₃](PF₆)₂, or [Os(1,10-Phenanthroline)₃](PF₆)₂.

12. The composition of claim 11, wherein the [Ru(2,2′-Bipyridine)₃](PF₆)₂ or [Os(2,2′-Bipyridine)₃](PF₆)₂ has a structure given by:

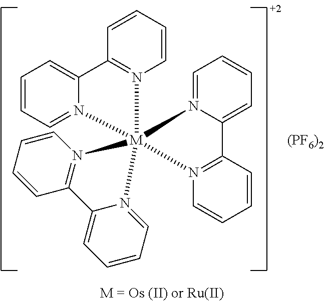

13. The composition of claim 1, wherein the metal based phosphor has a formula given by K₄[Ru(4,4′-dicarboxy-2,2′-bipyridine)₃], K₄[Os(4,4′-dicarboxy-2,2′-bipyridine)₃], [Ru(dicarboxy-1,10-Phenanthroline)₃](PF₆)₂, or [Os(dicarboxy-1,10-Phenanthroline)₃](PF₆)₂.

14. The composition of claim 13, wherein the K₄[Ru(4,4′-dicarboxy-2,2′-bipyridine)₃] or K₄[Os(4,4′-dicarboxy-2,2′-bipyridine)₃] has a structure given by:

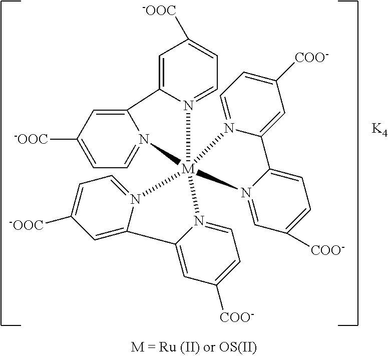

15. The composition of claim 1, wherein the metal based phosphor has a formula given by K₄[Ru(4,4′,4″-tricarboxy-2,2′:6′,2″-terpyridine)₂] or K₄[Ru(4,4′,4″-tricarboxy-2,2:6′,2″-terpyridine)₂].

16. The composition of claim 15, wherein the K₄[Ru(4,4′,4″-tricarboxy-2,2:6′,2″-terpyridine)₂] or K₄[Ru(4,4′,4″-tricarboxy-2,2:6′,2″-terpyridine)₂] has a structure given by:

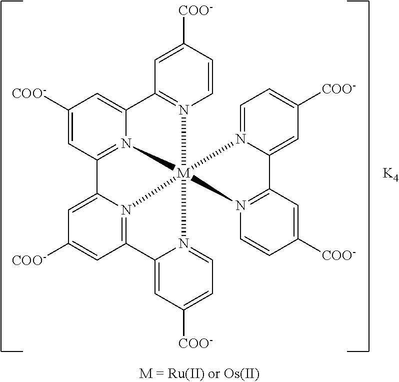

17. The composition of claim 1, wherein the metal based phosphor has a structure given by: 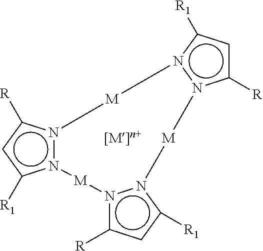
wherein R and R₁ are selected from hydrogen, alkyl, alkoxy, aryl, NH₂, NH₃⁺, COOH, COO⁻, SO₃⁻, SO₃⁻, PO₄H₂, PO₄²⁻, OH, Cl, Br, COOR′, or R′₃N⁺ wherein R′ is selected from hydrogen or alkyl, M comprises transition metals including gold, silver, copper, or combination thereof, and [M′]ⁿ⁺ comprises Ag⁺, Pb²⁺, Hg²⁺, and Tl³⁺.

18. The composition of claim 1, wherein the metal based phosphor has a structure given by: 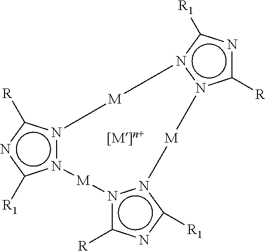
wherein R and R₁ are selected from hydrogen, alkyl, alkoxy, aryl, NH₂, NH₃⁺, COOH, COO⁻, SO₃H, SO₃⁻, PO₄H₂, PO₄²⁻, OH, Cl, Br, COOR′, or R′₃N⁺ wherein R′ is selected from hydrogen or alkyl, M comprises transition metals including gold, silver, copper, or combination thereof, and [M′]ⁿ⁺ comprises Ag⁺, Pb²⁺, Hg²⁺, and Tl³⁺.

19. The composition of claim 1, wherein the metal based phosphor has a structure given by: 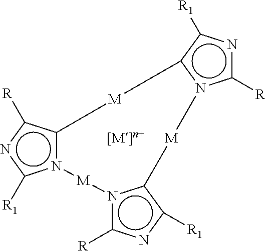
wherein R and R₁ are selected from hydrogen, alkyl, alkoxy, aryl, NH₂, NH₃⁺, COOH, COO⁻, SO₃H, SO₃⁻, PO₄H₂, PO₄²⁻, OH, Cl, Br, COOR′, or R′₃N⁺ wherein R′ is selected from hydrogen or alkyl, M comprises transition metals including gold, silver, copper, or combination thereof, and [m]ⁿ⁺ comprises Ag⁺, Pb²⁺, Hg²⁺, and Tl³⁺.

20. The composition of claim 1, wherein the matrix is poly-N-isopropylacrylamide (PNIPAM), chemically modified PNIPAM including PNIPAM-co-allylamine and PNIPAM-co-acrylic acid, or a combination thereof.

21. The composition of claim 1, wherein the matrix is chitosan, a chitosan derivative, a modified chitosan, or a combination thereof.

22. The composition of claim 21, wherein the chitosan derivatives comprise succinyl chitosan, octanoyl chitosan, quateraminated chitosan, caproyl chitosan, myristoyl chitosan, palmitoyl chitosan, chitosan thioglycolic acid, phosphorylated chitosan, carboxy methyl chitosan, and thiol containing chitosan.

23. The composition of claim 1, wherein the composition is used for biosensing and bioimaging, in anti-tumor therapy, for drug delivery, in biomedical devices, in live cell imaging, in environmental monitoring, in toxic metal removal, in small molecule detection, and for biological and chemical recognition.

24. The composition of claim 1, wherein the polymeric nano or microparticles have sizes ranging from 20 nm-1000 nm.

25. The composition of claim 1, wherein the polymeric nano or microparticles are incorporated in a delivery system along with biologically benign polymers, wherein the biologically benign polymers comprise one or more functional groups to reduce a toxicity, enhance a specificity or both.

26. The composition of claim 25, wherein the biologically benign polymers comprise chitosan, alginic acid, or combinations and modifications thereof.

27. The composition of claim 1, wherein a surface positive or negative charge of the composition is easily controllable by a selection and use of different polymers.

28. The composition of claim 1, wherein the surface charge of the composition controls a cellular uptake.

29. The composition of claim 1, wherein a particle size of the composition ranging from nano to micron size provides an enhanced cellular uptake by one or more mechanisms.

30. The composition of claim 1, wherein the luminescence of the polymeric nano or microparticles enhances a monitoring of the cellular uptake.

31. The composition of claim 1, wherein the composition comprises both hydrophilic and hydrophobic segments to entrap one or more photosensitizers, wherein the photosensitizers on light exposure are excited to a triplet state thereby leading to a generation of a singlet oxygen (¹O₂) or free radicals used for photodynamic therapy.

32. The composition of claim 1, wherein the composition has an emission maximum of 525 nm and an excitation maximum of 292 nm in aqueous solution.

33. The composition of claim 1, wherein the composition has an emission maximum of 500 nm and an excitation maximum of 356 nm in a solid state.

34. The composition of claim 1, wherein the composition comprising Pt(II) based phosphorescent polymeric nano/microparticles are used for oxygen sensing applications.

35. A stimulus-responsive water soluble phosphorescent system comprising:
a polyanionic metal based phosphor comprising [A^{x+}]ₙ₁ tris[tris(3,3′,3″-trisulfonatophenyl)phosphine]aurate(I) wherein [A^{x+}]nₙ₁, comprises [Na⁺]₈, [K⁺]₈, [Cs⁺]₈, [NH₄⁺]₈, [R₄N⁺]₈, [Mg⁺²]₄, [Ca⁺²]₄, or combination thereof, and tris[tris(3,3′,3″-trisulfonatophenyl)phosphine]aurate(I) having a structure given by 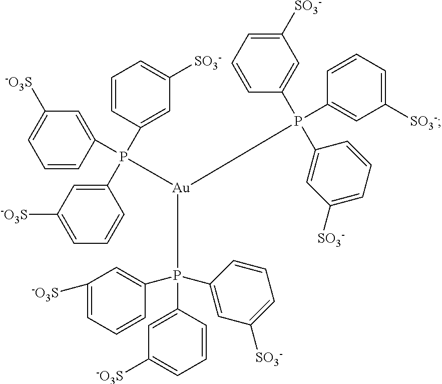
and
a stimulus-responsive matrix comprising poly-N-isopropylacrylamide (PNIPAM), chemically modified PNIPAM including PNIPAM-co-allylamine and PNIPAM-co-acrylic acid, chitosan, or a combination thereof, wherein the [A^{x+}]ₙ₁ tris[tris(3,3′,3″-trisulfonatophenyl)phosphine]aurate(I) is entrapped in the matrix to form of one or more luminescent polymeric nanoparticles or crosslinked to form polymeric micro/nanoparticles in absence of any chemical crosslinker.

36. The composition of claim 35, wherein the composition is responsive to one or more stimuli selected from the group consisting of pH, temperature, electric, magnetic, optical, and environmental stimuli.

37. The composition of claim 35, wherein the matrix is chitosan, a chitosan derivative, a modified chitosan, or combinations thereof.

38. The composition of claim 37, wherein the chitosan derivatives comprise succinyl chitosan, octanoyl chitosan, quateraminated chitosan, caproyl chitosan, myristoyl chitosan, palmitoyl chitosan, chitosan thioglycolic acid, phosphorylated chitosan, carboxy methyl chitosan, and thiol containing chitosan.

39. The composition of claim 35, wherein the composition is used for biosensing and bioimaging, in anti-tumor therapy, for drug delivery, in biomedical devices, in tissue scaffolds, for cellular encapsulation, in live cell imaging, in environmental monitoring, in toxic metal detection or removal, in small molecule detection, and for biological and chemical recognition.

40. The composition of claim 35, wherein there is no typical chemical crosslinking agent used along with a minimization of the use of harmful/toxic typical chemical crosslinkers.

41. The composition of claim 35, wherein the composition is a liquid at room temperature.

42. The composition of claim 35, wherein the composition comprising the PNIPAM matrix undergoes a transition from a liquid to a gel or an aggregate upon an increase in temperature.

43. A stimulus-responsive water soluble phosphorescent system comprising:
a Ru(II) or Os(II) based phosphor comprising a structure selected from the group consisting of 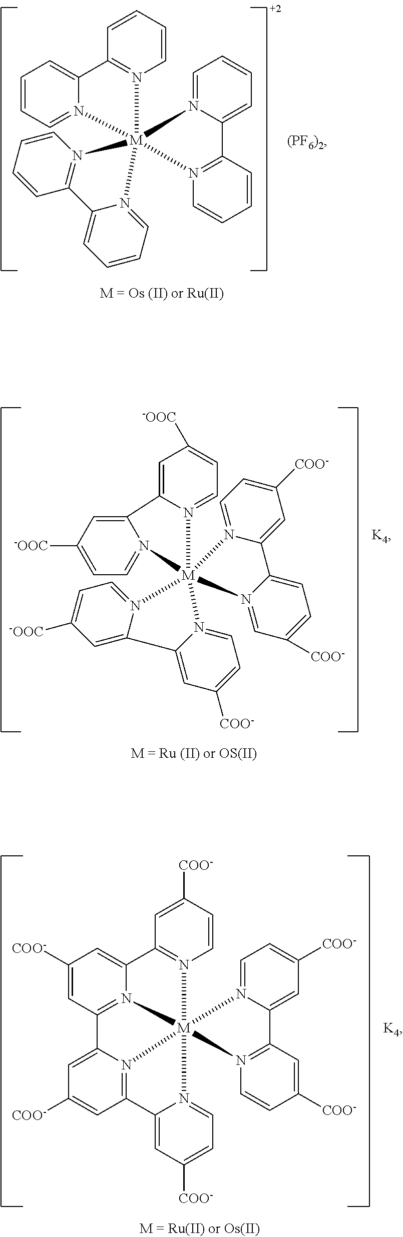
and combinations and modifications thereof, and
a stimulus-responsive matrix comprising poly-N-isopropylacrylamide (PNIPAM), chemically modified PNIPAM including PNIPAM-co-allylamine and PNIPAM-co-acrylic acid, or a combination thereof, chitosan, or a combination thereof, wherein the Ru(II) or Os(II) based phosphor is entrapped in the matrix to form of one or more luminescent polymeric nanoparticles or crosslinked to form polymeric micro/nanoparticles in absence of any chemical crosslinker.

44. The composition of claim 43, wherein the composition is responsive to one or more stimuli selected from the group consisting of pH, temperature, electric, magnetic, optical, and environmental stimuli.

45. The composition of claim 43, wherein the matrix is chitosan, a chitosan derivative, a modified chitosan, or combinations thereof.

46. The composition of claim 45, wherein the chitosan derivatives comprise succinyl chitosan, octanoyl chitosan, quateraminated chitosan, caproyl chitosan, myristoyl chitosan, palmitoyl chitosan, chitosan thioglycolic acid, phosphorylated chitosan, carboxy methyl chitosan, and thiol containing chitosan.

47. The composition of claim 43, wherein the composition is used for biosensing and bioimaging, in anti-tumor therapy, for drug delivery, in biomedical devices, in tissue scaffolds, for cellular encapsulation, in live cell imaging, in environmental monitoring, in toxic metal removal, in small molecule detection, and for biological and chemical recognition.

48. A method for making a stimulus-responsive hybrid luminescent or phosphorescent system comprising the steps of:
mixing an aqueous solution comprising a stimulus-responsive matrix comprising a polymer, a hydrogel, a colloid, a microgel, or combinations thereof with an aqueous solution of polyionic metal based phosphor with agitation to form a mixture in an inert atmosphere;
centrifuging the mixture with removal of a separated supernatant and a washing of a sediment at one or more regular intervals; and
forming the stimulus-responsive hybrid phosphorescent system by incubation of the centrifuged mixture in water, wherein the incubation results in a formation of one or more metal loaded microgel colloids or crystals.

49. The method of claim 48, comprising the optional steps of:
crosslinking the formed microgels by using one or more crosslinking agents to form a crosslinked microgel network; and
freeze drying the microgels or the microgel network.

50. The method of claim 48, wherein the metal based phosphors comprise anionic or cationic Au(I) or Pt(II) based phosphors in the form of a complex, a coordination compound, or combinations thereof.

51. The method of claim 48, wherein the matrix is poly-N-isopropylacrylamide (PNIPAM), chemically modified PNIPAM including PNIPAM-co-allylamine and PNIPAM-co-acrylic acid, or a combination thereof.

52. The method of claim 48, wherein the system is used for biosensing and bioimaging, in anti-tumor therapy, for drug delivery, in biomedical devices, in tissue scaffolds, for cellular encapsulation, in live cell imaging, in environmental monitoring, in toxic metal removal, in small molecule detection, and for biological and chemical recognition.

53. A method for making one or more luminescent or phosphorescent polyelectrolyte nano or microparticles comprising the steps of:
providing a polymer solution comprising modified or unmodified polymers wherein the polymers are selected from the group consisting of poly-N-isopropylacrylamide (PNIPAM), chemically modified PNIPAM including PNIPAM-co-allylamine and PNIPAM-co-acrylic acid, or a combination thereof, chitosan, chemically modified chitosan, chitosan derivatives poly acrylic acid (PAA), polyvinyl alcohol (PVA), alginic acid, PEG, modified PEG, agarose, hydroxy propyl cellulose, methyl methacrylate (MMA), hydroxyethyl methacrylate (HEMA), polystyrene, and poly-hydroxyethyl methacrylate;
adding a solution of one or more polyionic metal based phosphors to the polymer solution to form an opalescent suspension, wherein the metal based phosphors are entrapped in the polymer;
centrifuging the opalescent suspension; and
filtering the centrifuged suspension to recover the luminescent or phosphorescent polyelectrolyte nano or microparticles and separate any unreacted polymers or any unentrapped metal based phosphors.

54. The method of claim 53, wherein the polyionic metal based phosphors comprise anionic or cationic transition or noble metal based phosphors in the form of a complex, a coordination compound, or combinations thereof, wherein the metals are selected from the group consisting of gold, silver, copper, platinum, europium, terbium, ruthenium, rhenium, iridium, thallium, and osmium.

55. The method of claim 53, wherein the luminescent or phosphorescent polyelectrolyte nano or microparticles are used for biosensing and bioimaging, in anti-tumor therapy, for drug delivery, in biomedical devices, in tissue scaffolds, for cellular encapsulation, in live cell imaging, in environmental monitoring, in toxic metal removal, in small molecule detection, and for biological and chemical recognition.

56. A method for forming a polymer stabilized cyclic phosphorescent systems comprising the steps of:
mixing a solution of a cyclic ligand with a metal, metal complex or coordination compound in presence of an amine or a base with stirring to form a mixture, wherein the ligand comprises a modified or unmodified pyrazole, triazole, imidazole compound, or combinations and modifications thereof;
exposing the mixture to light, air or both;
following a progression of the reaction by monitoring a change in an emissive color of the mixture;
filtering and recrystallizing the solution at a completion of the reaction;
adding the filtered and recrystallized metal comprising cyclic ligand to a modified or unmodified polyionic polymer with stirring, wherein the polymer is a biopolymer, a thermosensitive polymer or both, wherein the metal comprising cyclic ligand is entrapped and stabilized in the polymer; and
recovering a polymer stabilized cyclic phosphorescent system by centrifugation.

57. A method for forming a polymer stabilized cyclic phosphorescent systems comprising the steps of:
adding a solution of a cyclic ligand to a modified or unmodified polyionic polymer with stirring at a controlled pH to form a mixture, wherein the pH is controlled by an addition of an acid or a base, wherein the ligand comprises a modified or unmodified pyrazole, triazole, imidazole compound, or combinations and modifications thereof, wherein the polymer is a biopolymer, a thermosensitive polymer, or both;
adding a metal, metal complex or coordination compound with stirring to the mixture;
following a progression of the reaction by monitoring a change in an emissive color of the mixture; and
centrifuging the mixture at a completion of the reaction to separate any unreacted polymer or the metal and to recover the polymer stabilized cyclic phosphorescent system in the sediment.

58. The method of claim 57, wherein the metal comprises gold, silver, platinum, or copper.

59. The method of claim 57, wherein the system is used for monitoring or detecting a level of one or more toxic heavy metal contaminants in a liquid sample.

60. A method of treating a cancer in a human subject comprising the steps of:
identifying the human subject in need for the treatment of the cancer; and
administering a therapeutically effective amount of a composition comprising one or more polyanionic or polycationic metal based phosphors in the form of a complex, a coordination compound, or combinations thereof linked, attached or entrapped in a stimulus-responsive matrix comprising a polymer, a hydrogel, a colloid, a softgel, a microgel, or combinations thereof in an amount sufficient to treat the cancer, wherein the composition is administered alone, as a combination with other anticancer drugs, or in conjunction with a chemotherapeutic or radiation regimen.

61. The method of claim 60, further comprising the optional steps of:
measuring or monitoring a photoluminescent signal emanating from the metal based phosphors to follow an uptake of the composition into one or more normal or cancerous cells; and
increasing a local concentration of the composition or the combination anticancer drug by inducing an aggregation, a gelation or both of the composition by providing localized heating or a hyperthermia treatment.

62. The method of claim 60, wherein the composition has a gelation temperature at or about a body temperature.

63. A method for monitoring or detecting a level of one or more volatile organic compounds (VOCs), carcinogens, and other contaminants in a sample comprising the steps of:
providing the sample suspected of having the VOCs, carcinogens, or other contaminants, wherein the sample is a liquid or a gas, and comprises water, biological fluids, air, or a mixture of gases;
providing a photoluminescent composition comprising one or more polyanionic or polycationic metal based phosphors in the form of a complex, a coordination compound, or combinations thereof linked, attached or entrapped in a stimulus-responsive matrix comprising a polymer, a hydrogel, a colloid, a softgel, or a microgel;
measuring a baseline photoluminescent signal following contact of the composition with a pure sample, wherein the pure sample does not have the VOCs, carcinogens, or other contaminants;
measuring the photoluminescent signal following contact of the composition with the sample suspected of having the VOCs, carcinogens, or other contaminants; and
monitoring or detecting the level of one or more volatile organic compounds (VOCs), carcinogens, and other contaminants by measuring a change in an intensity and a magnitude of the photoluminescent signal induced by a change in a pH or a temperature of the sample by the one or more volatile organic compounds (VOCs), carcinogens, and other contaminants.

64. A method for monitoring or detecting a level of one or more toxic heavy metal contaminants in a liquid sample comprising the steps of:
providing the liquid sample suspected of having the toxic metal contamination, wherein the liquid sample comprises drinking water, biological fluids, industrial effluents, ground water, wastewater, or combinations thereof;
providing a photoluminescent composition comprising one or more polyanionic or polycationic metal based phosphors in the form of a complex, a coordination compound, or combinations thereof linked, attached or entrapped in a stimulus-responsive matrix comprising a polymer, a hydrogel, a colloid, a softgel, or a microgel;
monitoring an emission color of the composition prior to contacting the composition with the liquid sample, wherein the monitoring is done under daylight, UV light, or both;
monitoring the emission color after contacting the composition with the liquid sample, wherein the monitoring is done under daylight, UV light, or both;
monitoring or detecting the level of one or more toxic metal contaminants in a liquid sample based on a change in the emission color induced by an entrapment or a change in a pH of the one or more toxic heavy metal contaminants by the composition; and
monitoring an energy dispersive X-ray analysis (EDX) spectrum of a matrix/polymeric particle system formed after contacting the composition with the liquid contaminant sample enabling a qualitative and a quantitative determination heavy metal contaminant.

65. The method of claim 64, wherein the one or more toxic heavy metal contaminants comprise, lead, mercury, thallium, arsenic, copper, and silver.

66. The method of claim 64, wherein the polymers may have a film forming ability thereby enabling manufacture of a luminescent pH film with a pH sensitive emission profile.

67. A method for in vivo imaging one or more live cells, tissues, or both, targeting one or more receptors, cells, or tissues or detecting a level of a protein, a molecular marker, or a biomolecule comprising the steps of:
providing an animal or a human subject;
injecting a photoluminescent composition comprising one or more polyanionic or polycationic metal based phosphors in the form of a complex, a coordination compound, or combinations thereof linked, attached or entrapped in a stimulus-responsive matrix comprising a polymer, a hydrogel, a colloid, a softgel, or a microgel; and
monitoring a photoluminescent signal following the injection to image the one or more live cells, tissues, or both, target the one or more receptors, cells, or tissues, or to detect a level of the protein, the molecular marker, or the biomolecule.

68. The method of claim 67, wherein the photoluminescent composition is optionally tagged, linked or conjugated with one or more antibodies, organic dyes, ligands, or nanoparticles.

69. The method of claim 67, wherein an enhanced photoluminescence signal from a composition complex in presence of a stimuli responsive matrix can minimize concentration of dye or overcome an auto fluorescence signal from the one or more live cells, tissues, receptors, or any other biological matrix.
